(19) United States
(12) Patent Application Publication
(10) Pub. No.: US 2026/0158074 A1
(43) Pub. Date: Jun. 11, 2026

(54) A Cell Surface Marker Signature for Arrhythmogenic Pluripotent Stem Cell-Derived Cardiomyocytes

(71) Applicants: Western Sydney Local Health District, Westmead, New South Wales (AU); The University of Sydney, Sydney, New South Wales (AU); The University of Queensland, St. Lucia, Queensland (AU)

(72) Inventors: James Chong, Sydney, New South Wales (AU); Zoe Clayton, Sydney, New South Wales (AU); Dinesh Selvakumar; Jacob George, Sydney, New South Wales (AU); Leila Reyes, Sydney, New South Wales (AU); Steve Dingwall, St. Lucia, Queensland (AU)

(21) Appl. No.: 18/872,451

(22) PCT Filed: Jun. 8, 2023
(86) PCT No.: PCT/AU2023/050503

§ 371 Date: Dec. 6, 2024

(30) Foreign Application Priority Data
Jun. 8, 2022 (AU) 2022901568

(51) Int. Cl. A61K 35/34; A61P 9/06; G01N 33/569

(52) U.S. Cl. CPC A61K 35/34; A61P 9/06; G01N 33/56966; G01N 2333/70596

(57) ABSTRACT

Disclosed herein is a method of identifying an arrhythmogenic pluripotent stem cell-derived cardiomyocyte (PSC-CM), the method comprising: (i) determining whether CD200 is expressed on a surface of the PSC-CM; wherein if CD200 is expressed on the surface of the PSC-CM, the PSC-CM is an arrhythmogenic PSC-CM.

### FIELD

The present invention relates to methods of identification of subpopulations of cardiomyocytes, particularly human pluripotent stem cell-derived cardiomyocytes, such as arrhythmogenic, pacemaker or ventricular cardiomyocytes.

### BACKGROUND

Myocardial infarction (MI), the leading cause of heart failure, results in the loss of up to 1 billion highly specialised cardiomyocytes. Though substantial breakthroughs have advanced cardiovascular medicine in recent decades, heart disease remains responsible for more deaths worldwide than any other illness, an issue compounded by the inability of the injured adult heart to meaningfully regenerate. Despite great interest, numerous attempts to replace damaged or destroyed cardiomyocytes with exogenous cells have yielded inconsistent results. Most cell types that have been clinically tested lack robust capacity to differentiate into functional cardiomyocytes and exert any beneficial effects through paracrine mechanisms rather than remuscularisation. In contrast, pluripotent stem cells (PSCs) have demonstrated cardiomyogenic differentiation capacity, potentially providing a limitless source of cardiomyocytes for therapeutic applications. Exciting pre-clinical data have confirmed that engrafted PSC derived cardiomyocytes (PSC-CMs) can remuscularise and improve cardiac function in clinically relevant large animal MI models. However, hurdles to widescale clinical translation remain. The most concerning of these relates to the potentially lethal cardiac rhythm disturbances which ensue following intramyocardial PSC-CM delivery, hereafter referred to as engraftment arrhythmias (EA).

There have been few studies examining the potential mechanism of EA, with existing evidence from electrophysiological studies suggesting that EAs originate from PSC-CM grafts with characteristics of abnormal impulse generation and enhanced automaticity. Similar to the lack of data on EA mechanism, there has only been one study on therapeutic strategies to mitigate EAs, suggesting that pharmacotherapy (with amiodarone and ivabradine) can suppress but not abolish EA.

There is therefore a need for methods to reduce or eliminate EA following intramyocardial PSC-CM delivery.

### SUMMARY OF INVENTION

In a first aspect of the invention, there is provided a method of identifying an arrhythmogenic pluripotent stem cell-derived cardiomyocyte (PSC-CM), the method comprising:
(i) determining whether CD200 is expressed on a surface of the PSC-CM; wherein if CD200 is expressed on the surface of the PSC-CM, the PSC-CM is an arrhythmogenic PSC-CM.

The following options may be used in conjunction with the first aspect of the invention, either individually or in any combination.

The method of the first aspect of the invention may further comprise:
(ii) determining whether Signal regulatory protein α (CD172a) is expressed on the surface of the PSC-CM, and/or
(iii) determining whether CD90 is expressed on the surface of the PSC-CM; wherein if CD200 is expressed on a surface of the PSC-CM, and CD172a is expressed on the surface of the PSC-CM and/or CD90 is not expressed on the surface of the PSC-CM, the PSC-CM is an arrhythmogenic PSC-CM.

The method of the first aspect of the invention may further comprise:
(ii) determining whether CD172a is expressed on the surface of the PSC-CM, and
(iii) determining whether CD90 is expressed on the surface of the PSC-CM; wherein if CD200 and CD172a are expressed on the surface of the PSC-CM and CD90 is not expressed on the surface of the PSC-CM, the PSC-CM is an arrhythmogenic PSC-CM.

If CD200 is not expressed on the surface of the PSC-CM, the PSC-CM may be a non-arrhythmogenic PSC-CM.

Determining whether CD200, CD172a and/or CD90 is expressed on the surface of the PSC-CM may comprise exposing the PSC-CM to an agent comprising a detectable label to provide a labelled PSC-CM, and detecting the detectable label, wherein the agent selectively binds to CD200, CD172a or CD90. Determining whether CD200, CD172a and CD90 are expressed on the surface of the PSC-CM may comprise exposing the PSC-CM to a first agent comprising a first detectable label that selectively binds to CD200, a second agent comprising a second detectable label that selectively binds to CD172a, and a third agent comprising a third detectable label that selectively binds to CD90, to provide a labelled PSC-CM, and detecting the detectable labels.

The agent comprising a detectable label may be an antibody, and the detectable label may be a fluorophore. Detecting the detectable label may comprise subjecting the labelled hPSC-CM to flow cytometry.

The PSC-CM may be a human pluripotent stem cell-derived cardiomyocyte (hPSC-CM).

In a second aspect of the invention, there is provided a method of determining whether a dose containing a plurality of PSC-CMs is likely to cause arrhythmia upon transplantation in a subject, the method comprising:
(i) determining whether the dose contains arrhythmogenic PSC-CMs, wherein whether a PSC-CM is an arrhythmogenic PSC-CM is determined according to the method of the first aspect of the invention;
wherein if the dose contains arrhythmogenic PSC-CM's, the dose is likely to cause arrhythmia upon transplantation in the subject.

The following options may be used in conjunction with the second aspect of the invention, either individually or in any combination.

The method of the second aspect of the invention may further comprise:
(ii) determining the proportion of arrhythmogenic PSC-CMs in the dose,
wherein if the proportion of PSC-CMs in the dose which express CD200 on a surface thereof is greater than 0.001%, the dose is likely to cause arrhythmia upon transplantation in the subject.

In a third aspect of the invention, there is provided a method of eliminating or reducing arrhythmia following transplantation of a dose containing a plurality of PSC-CMs in a subject, the method comprising, prior to transplantation of the dose:
(i) identifying arrhythmogenic PSC-CMs contained in a crude dose according to the method of the first aspect of the invention; and
(ii) removing the arrhythmogenic PSC-CMs from the crude dose to obtain a purified dose.

The following options may be used in conjunction with the third aspect of the invention, either individually or in any combination.

The method of the third aspect of the invention may further comprise:
(iii) transplanting the purified dose in the subject.

The crude dose may comprise pluripotent stem cells (PSCs) which have been differentiated to form PSC-CMs, and have not been subject to any additional treatments enriching or depleting a cell subpopulation.

Arrhythmia following transplantation of the purified dose may be reduced compared to arrhythmia following transplantation of the crude dose. Arrhythmia may be reduced as defined by cumulative time per day spent in arrhythmia over 25 days post-transplantation. Arrhythmia may be reduced by at least about 50%, or at least about 60, 70, 80, 90, 95, or 100%.

Step (ii) may comprise removing at least about 50% of the arrhythmogenic PSC-CMs from the crude dose. Step (ii) may comprise removing at least about 60%, or at least about 70, 80, 90, 95, or 100% of the arrhythmogenic PSC-CMs from the crude dose

Step (ii) may be performed by fluorescence-activated cell sorting or by magnetic-activated cell sorting.

In a fourth aspect of the invention, there is provided a method of providing a dose containing a plurality of PSC-CM which is substantially free from arrhythmogenic PSC-CMs, the method comprising:
(i) identifying arrhythmogenic PSC-CMs contained in a crude dose according to the method of the first aspect of the invention; and
(ii) removing the arrhythmogenic PSC-CMs from the crude dose to obtain a dose of PSC-CM which is substantially free from arrhythmogenic PSC-CMs.

The following options may be used in conjunction with the fourth aspect of the invention, either individually or in any combination.

The crude dose may comprise human pluripotent stem cells (PSCs) which have been differentiated to form PSC-CMs, and have not been subject to any additional treatments enriching or depleting a cell subpopulation.

Step (ii) may comprise removing at least about 50% of the arrhythmogenic PSC-CMs from the crude dose. Step (ii) may comprise removing at least about 60%, or at least about 70, 80, 90, 95, or 100% of the arrhythmogenic PSC-CMs from the crude dose.

The dose of PSC-CM which is substantially free from arrhythmogenic PSC-CMs may contain less than 0.001% of arrhythmogenic PSC-CMs, relative to the total number of PSC-CM in the dose.

Step (ii) may be performed by fluorescence-activated cell sorting or by magnetic-activated cell sorting.

In a fifth aspect of the invention, there is provided a method of identifying a pluripotent stem cell-derived cardiac cell with pacemaker properties (PSC-PM) the method comprising:
(i) determining whether CD200 is expressed on a surface of a pluripotent stem cell-derived cardiac cell;
wherein if CD200 is expressed on the surface of the pluripotent stem cell-derived cardiac cell, the pluripotent stem cell-derived cardiac cell is a PSC-PM.

The following options may be used in conjunction with the fifth aspect of the invention, either individually or in any combination.

The method of the fifth aspect of the invention may further comprise:
(ii) determining whether CD172a is expressed on the surface of the pluripotent stem cell-derived cardiac cell, and/or
(iii) determining whether CD90 is expressed on the surface of the pluripotent stem cell-derived cardiac cell;
wherein if CD200 is expressed on a surface of the pluripotent stem cell-derived cardiac cell, and CD172a is expressed on the surface of the pluripotent stem cell-derived cardiac cell and/or CD90 is not expressed on the surface of the pluripotent stem cell-derived cardiac cell, the pluripotent stem cell-derived cardiac cell is a pacemaker PSC-PM.

The method of the fifth aspect of the invention may further comprise:
(ii) determining whether CD172a is expressed on the surface of the pluripotent stem cell-derived cardiac cell, and
(iii) determining whether CD90 is expressed on the surface of the pluripotent stem cell-derived cardiac cell;
wherein if CD200 and CD172a are expressed on the surface of the pluripotent stem cell-derived cardiac cell and CD90 is not expressed on the surface of the pluripotent stem cell-derived cardiac cell, the pluripotent stem cell-derived cardiac cell is a PSC-PM.

If CD200 is not expressed on the surface of the pluripotent stem cell-derived cardiac cell, the pluripotent stem cell-derived cardiac cell may not be a PSC-PM.

Determining whether CD200, CD172a and/or CD90 is expressed on the surface of the pluripotent stem cell-derived cardiac cell may comprise exposing the pluripotent stem cell-derived cardiac cell to an agent comprising a detectable label to provide a labelled pluripotent stem cell-derived cardiac cell, and detecting the detectable label, wherein the agent selectively binds to CD200, CD172a or CD90. Determining whether CD200, CD172a and CD90 are expressed on the surface of the pluripotent stem cell-derived cardiac cell may comprise exposing the pluripotent stem cell-derived cardiac cell to a first agent comprising a first detectable label that selectively binds to CD200, a second agent comprising a second detectable label that selectively binds to CD172a, and a third agent comprising a third detectable label that selectively binds to CD90, to provide a labelled pluripotent stem cell-derived cardiac cell, and detecting the detectable labels.

The agent comprising a detectable label may be an antibody, and the detectable label may be a fluorophore. Detecting the detectable label may comprise subjecting the labelled pluripotent stem cell-derived cardiac cell to flow cytometry.

In a sixth aspect of the invention, there is provided a method of providing a substantially pure dose of PSC-PM, the method comprising:
(i) identifying PSC-PMs contained in a plurality of pluripotent stem cell-derived cardiac cells according to the method of the fifth aspect of the invention; and
(ii) isolating the PSC-PMs from the plurality of pluripotent stem cell-derived cardiac cells to obtain the substantially pure dose of PSC-PM.

The following options may be used in conjunction with the sixth aspect of the invention, either individually or in any combination.

Step (ii) may be performed by fluorescence-activated cell sorting or by magnetic-activated cell sorting.

The substantially pure dose of PSC-PM may contain less than 0.001% of cells which are not PSC-PM, relative to the total number of cells in the dose.

In a seventh aspect of the invention, there is provided a method of identifying a pluripotent stem cell-derived ventricular cardiomyocyte (PSC-VM), the method comprising:
(i) determining whether CD200 is expressed on a surface of a pluripotent stem cell-derived cardiac cell;
wherein if CD200 is not expressed on the surface of the pluripotent stem cell-derived cardiac cell, the pluripotent stem cell-derived cardiac cell is a PSC-VM.

The following options may be used in conjunction with the seventh aspect of the invention, either individually or in any combination.

The method of the seventh aspect of the invention may further comprise:
(ii) determining whether CD172a is expressed on the surface of the pluripotent stem cell-derived cardiac cell, and/or
(iii) determining whether CD90 is expressed on the surface of the pluripotent stem cell-derived cardiac cell;
wherein if CD200 is not expressed on a surface of the pluripotent stem cell-derived cardiac cell, and CD172a is expressed on the surface of the pluripotent stem cell-derived cardiac cell and/or CD90 is not expressed on the surface of the pluripotent stem cell-derived cardiac cell, the pluripotent stem cell-derived cardiac cell is a PSC-VM.

The method of the seventh aspect of the invention may further comprise:
(ii) determining CD172a is expressed on the surface of the pluripotent stem cell-derived cardiac cell, and
(iii) determining whether CD90 is expressed on the surface of the pluripotent stem cell-derived cardiac cell;
wherein if CD200 and CD90 are not expressed on the surface of the pluripotent stem cell-derived cardiac cell and CD172a is expressed on the surface of the pluripotent stem cell-derived cardiac cell, the pluripotent stem cell-derived cardiac cell is a PSC-VM.

If CD200 is expressed on the surface of the pluripotent stem cell-derived cardiac cell, the pluripotent stem cell-derived cardiac cell may not be a PSC-VM.

Determining whether CD200, CD172a and/or CD90 is expressed on the surface of the pluripotent stem cell-derived cardiac cell may comprise exposing the pluripotent stem cell-derived cardiac cell to an agent comprising a detectable label to provide a labelled pluripotent stem cell-derived cardiac cell, and detecting the detectable label, wherein the agent selectively binds to CD200, CD172a or CD90. Determining whether CD200, CD172a and CD90 are expressed on the surface of the pluripotent stem cell-derived cardiac cell may comprise exposing the pluripotent stem cell-derived cardiac cell to a first agent comprising a first detectable label that selectively binds to CD200, a second agent comprising a second detectable label that selectively binds to CD172a, and a third agent comprising a third detectable label that selectively binds to CD90, to provide a labelled pluripotent stem cell-

The agent comprising a detectable label may be an antibody, and the detectable label may be a fluorophore. Detecting the detectable label may comprise subjecting the labelled pluripotent stem cell-derived cardiac cell to flow cytometry.

In an eighth aspect of the invention, there is provided a method of providing a substantially pure dose of PSC-VM, the method comprising:
(i) identifying PSC-VM contained in a plurality of pluripotent stem cell-derived cardiac cells according to the method of the seventh aspect of the invention; and
(ii) isolating the PSC-VM from the plurality of pluripotent stem cell-derived cardiac cells to obtain the substantially pure dose of PSC-VM.

The following options may be used in conjunction with the eighth aspect of the invention, either individually or in any combination.

Step (ii) may be performed by fluorescence-activated cell sorting or by magnetic-activated cell sorting.

The substantially pure dose of PSC-PM may contain less than 0.001% of cells which are not PSC-PM, relative to the total number of cells in the dose.

In a ninth aspect of the invention, there is provided a method of eliminating or reducing arrhythmia following transplantation of a dose containing a plurality of PSC-CMs in a subject, the method comprising culturing a plurality of PSCs in a culture medium under conditions effective to suppress the generation of arrhythmogenic PSC-CMs, wherein an arrhythmogenic PSC-CM is a PSC-CM which expresses CD200 on a surface thereof.

The following options may be used in conjunction with the ninth aspect of the invention, either individually or in any combination.

Arrhythmia following transplantation of a dose subjected to the method of the ninth aspect of the invention may be reduced compared to arrhythmia following transplantation of a dose which is not subjected to the method of the ninth aspect of the invention. Arrhythmia may be reduced as defined by cumulative time per day spent in arrhythmia over 25 days post-transplantation. Arrhythmia may be reduced by at least about 50%, or at least about 60, 70, 80, 90, 95, or 100%.

In a tenth aspect of the invention, there is provided a method of providing a dose of PSC-CM which is substantially free from arrhythmogenic PSC-CMs, the method comprising culturing PSCs in a culture medium under conditions effective to suppress the generation of arrhythmogenic PSC-CMs, wherein an arrhythmogenic PSC-CM is a PSC-CM which expresses CD200 on a surface thereof.

The following options may be used in conjunction with the tenth aspect of the invention, either individually or in any combination.

The dose of PSC-CM which is substantially free from arrhythmogenic PSC-CMs may contain less than 0.001% of arrhythmogenic PSC-CMs, relative to the total number of PSC-CM in the dose.

An arrhythmogenic PSC-CMs may be a PSC-CM which expresses CD200, and expresses CD172a and/or does not express CD90, on the surface thereof. An arrhythmogenic PSC-CM may be a PSC-CM which expresses CD200 and CD172a, and does not express CD90, on the surface thereof.

In an eleventh aspect of the invention, there is provided a method of providing a dose of PSC-CM which contains an increased proportion of non-arrhythmogenic PSC-CMs, the method comprising culturing PSCs in a culture medium under conditions effective to increase the generation of non-arrhythmogenic PSC-CMs, wherein a non-arrhythmogenic PSC-CM is a PSC-CM which does not express CD200 on a surface thereof.

The following options may be used in conjunction with an eleventh aspect of the invention, either individually or in any combination.

The dose of PSC-CM which contains an increased proportion of non-arrhythmogenic PSC-CMs may contain at least about 5% more non-arrhythmogenic PSC-CMs, relative to the total number of PSC-CM in the dose, and compared to a control dose which is obtained by culturing PSCs in a culture medium in the absence of conditions effective to increase the generation of non-arrhythmogenic PSC-CMs.

A non-arrhythmogenic PSC-CMs may be a PSC-CM which does not express CD200, and does not express CD90 and/or expresses CD172a, on the surface thereof. A non-arrhythmogenic PSC-CM may be a PSC-CM which does not express CD200 and CD90, and expresses CD172a on the surface thereof.

The conditions effective to suppress the generation of arrhythmogenic PSC-CMs may comprise addition of a retinoic acid inhibitor to the culture medium or removal of vitamin A from the culture medium.

The retinoic acid inhibitor may be added during cardiomyocyte differentiation. The retinoic acid inhibitor may be added at between day 3 to day 7 of cardiomyocyte differentiation.

The retinoic acid inhibitor may be selected from the group consisting of 4-diethylaminobenzaldehyde (DEAB), 4-[(1E)-2-[5,6-dihydro-5,5-dimethyl-8-(2-phenylethynyl)-2-naphthalenyl]ethenyl]benzoic acid (BMS-493), (E)-4-[2-(5,6-dihydro-5,5-dimethyl-8-phenyl-2-naphthalenyl)ethenyl]-benzoic acid (BMS-189453), and disulfiram.

Vitamin A may be removed from the culture medium during cardiomyocyte differentiation.

### BRIEF DESCRIPTION OF DRAWINGS

FIG. 1. Schematic depicting PSC-CM stirred tank reactor production protocol.

FIG. 2. High parameter flow cytometry analysis workflow. (a) Steps involved in preparing data, (b) generating tSNE plot and overlaying with FlowSOM clusters, and (c) identifying and quantifying subpopulations.

FIG. 3. Focal, automatic engraftment arrhythmias can be effectively suppressed with pharmacotherapy. (a) Study timeline for phase 1 large animal experiments in which 13 swine were randomised to 4 treatment groups (PSC-CM, PSC-CM+AA, Vehicle) following percutaneously induced myocardial infarction. A further 2 animals underwent sham infarction and vehicle injection. (b) Representative CMR reconstruction (created with ADAS 3D) merged with epicardial voltage map, used to choose, and annotate border and infarct zone injection sites (black circles). (c) Plot depicting the percentage of time per day each cell (red), cell and drug (blue), or vehicle (green) recipient spent in ventricular arrhythmia over course of study protocol. (d) Representative endocardial activation map during EA localising arrhythmia origin to focal sites of cell injections (white: early activation, purple: late activation, star: site of earliest activation) (e) Representative EA persisting through burst pacing suggesting an automatic rather than re-entrant mechanism. (f) Dose-dependent suppression of PSC-CM contraction rate using amiodarone (dark purple, square) and ivabradine (light purple, circle) in vitro (0-100 μM drug concentrations). (g) Telemetry strip from representative cell recipient exhibiting run of EA with spontaneous onset and offset. (h) Telemetry strip from representative cell and anti-arrhythmic recipient showing predominantly sinus rhythm with occasional ventricular ectopic beat. (i-k) Arrythmia parameters from day 0 to day 28 for each subject, grouped by treatment allocation (presented as mean±SEM). Significant reduction in (i) total hours of arrythmia (*p=0.03, Mann Whitney test), (j) days with arrhythmia (** p=0.003, unpaired t-test, df=6), and (k) peak arrhythmia heart rate (* p=0.02, unpaired t-test, df=6) in cell recipients treated with anti-arrhythmics.

FIG. 4. Effects of PSC-CM transplantation on cardiac volumes and function. (a) Representative short-axis cine MRI from end-diastolic and end-systolic phases of the cardiac cycle 4 weeks following cell or vehicle injection. Greater ejection of blood in systole for cell compared to vehicle recipients most notably in cell recipients treated with anti-arrhythmic drugs. (b) Plot depicting the change in scar size from baseline (post-MI, pre-injection) to 4-week post-injection, with no significant difference between treatment groups (p>0.99, Kruskal-Wallis test) (c) Plot depicting the change in LVEF from baseline (post-MI, pre-injection) to 4-week post-injection, with significantly improved LVEF in cell and anti-arrhythmic recipients (Vehicle vs PSC-CM: p=0.27; Vehicle vs PSC-CM+AA: * p=0.03; Kruskal-Wallis with Dunn's multiple comparisons test). (d-g) Pooled group data of left and right ventricular function at baseline (post-MI, pre-injection) and 4 weeks post-injection. Greatest improvement in (d) LVEF was noted in cell and anti-arrhythmic recipients. No significant change in (e) LVEDV observed between groups (p=0.38, Kruskal-Wallis test) though there was significant improvement in (f) LVSV in the cell and anti-arrhythmic recipients (Vehicle vs PSC-CM: p=0.69; Vehicle vs PSC-CM+AA: * p=0.03; Kruskal-Wallis with Dunn's multiple comparisons test). For (g) RVEF there was no significant difference between groups however a trend to improvement with cell recipients (p=0.07, Kruskal-Wallis Test).

FIG. 5. PSC-CM cell doses are heterogeneous with arrhythmogenic subpopulations. (a) UMAP embedding of scRNA-seq data from representative samples of cell doses. 10 clusters were identified and annotated based on differential gene expression. (b) Dot plot showing expression of representative marker genes used to categorise clusters. (c) Nebulosa plots showing the density of specific marker gene expression based on gene-weighted density estimation, demarcating cluster identities. (d) Representative tSNE plot from sample of a cell dose analysed by flow cytometry. FlowSOM metaclusters overlaid onto the tSNE highlight several discrete subpopulations within the cell fraction which were identified based on surface marker signatures. (e) Heat map showing relative expression of cell surface markers within each metacluster. (f) Nebulosa plots displaying expression levels of CD172a, CD200 and CD90. Right-most panel outlining CD172a⁺CD90⁻CD200⁻ cardiomyocytes which were negatively associated with arrhythmias, and CD172a⁺CD90⁻CD200⁺ cardiomyocytes which were positively associated with arrhythmias. Arrhythmogenic CD172a⁺CD90⁻CD200⁺ cardiomyocytes isolate to atrial and pacemaker cardiomyocytes (cluster 1) and non-arrhythmogenic CD172a⁺CD90⁻ CD200⁻ cardiomyocytes encompass all remaining cardiomyocyte clusters (clusters 0, 2, 3, 4).

FIG. 6. RA-PSC-CMs are enriched with atrial and pacemaker subpopulations and are highly arrhythmogenic. (a) Bar plot showing the contribution of different clusters to cells from representative PSC-CM and RA-PSC-CM cell doses. Proportionally greater contribution of compact ventricular cardiomyocytes in PSC-CM cell dose compared to greater proportion of atrial and pacemaker cardiomyocytes in RA-PSC-CM cell dose. (b) UMAP plot grouped by cells derived from either PSC-CM cell doses (red) or RA-PSC-CM cell doses (purple). RA-PSC-CM cell doses were predominantly made up of cluster 1 cells, atrial and pacemaker cardiomyocytes. (c) Analysis of sodium current densities in PSC-CMs and RA-PSC-CMs. Left—plot depicting maximum current densities recorded at −20 mV in PSC-CMs (n=35) and RA-PSC-CMs (n=16). Significantly lower current densities in RA-PSC-CMs (* p=0.02, Mann Whitney test). Right—Representative recordings of sodium current in PSC-CMs and RA-PSC-CMs made at different membrane potentials (inset: voltage protocol). (d) Left—Plot depicting action potential duration at 90% depolarisation (APD90) in PSC-CMs (n=68) and RA-PSC-CMs (n=85). Significantly shorter APD90 in RA-PSC-CMs (**** p<0.0001, Mann Whitney test). Right—Representative appearance of action potential morphology and spontaneous beat rates from PSC-CMs and RA-PSC-CMs. (e) Pooled data expressing percentage of time spent in ventricular arrhythmia and (f) mean heart rate per day between groups (mean±SEM). Substantially faster and more abundant arrhythmias in RA-PSC-CM group (*RA-PSC-CM n=3 only from day 0-day 10, n=1 from day 11-day 20. RA Pig #1 died on day 20, RA Pig #2 underwent catheter ablation on day 10 with data excluded thereafter, RA Pig #3 died on day 10). (g) High-parameter flow cytometric analysis comparing representative PSC-CM and RA-PSC-CM cell doses. Left: Concatenated tSNE plot showing distribution of cells from PSC-CM dose (red) and RA-PSC-CM dose (purple). Right: Concatenated tSNE plot showing distribution of CD172a⁺CD90⁻CD200⁺ and CD172a⁺CD90⁻CD200⁻ cardiomyocytes. Arrhythmogenic CD172a⁺CD90⁻CD200⁺ cardiomyocytes predominantly belong to RA-PSC-CM cell dose. Conversely non-arrhythmogenic CD172a⁺CD90⁻CD200⁻ cardiomyocytes predominantly belong to PSC-CM cell dose. (h) Plot depicting proportion of CD172a⁺CD90⁻CD200⁺ and CD172a⁺CD90⁻CD200⁻ cardiomyocytes in each PSC-CM and RA-PSC-CM cell dose. Significantly increased proportion of arrhythmogenic CD172a⁺CD90⁻CD200⁺ and reduced proportion of CD172a⁺CD90⁻CD200⁻ cardiomyocytes in RA-PSC-CM doses (* p=0.02, *** p=0.0001, unpaired t-test, df=5) (i) Correlation between total hours of arrhythmia from day 0-day 10 and proportion of arrhythmogenic CD172a⁺CD90⁻CD200⁺ cardiomyocytes or (j) non-arrhythmogenic CD172a⁺CD90⁻CD200⁻ cardiomyocytes in PSC-CM (red) and RA-PSC-CM (purple) cell doses. Significant positive (r=0.92, p=0.003) and negative linear correlations (r=−0.90, p=0.006). (k) Total daily activity between groups as measured in G-forces by accelerometers in implanted telemetry units. No significant difference in total activity between PSC-CM or PSC-CM+AA groups compared to vehicle, however RA-PSC-CM subjects had significantly lower daily activity (Vehicle vs PSC-CM: p=0.24; Vehicle vs PSC-CM+AA: p>0.99; Vehicle vs RA-PSC-CM: **** p<0.0001; Ordinary one-way ANOVA with Dunnett's multiple comparisons test) (1) Kaplan-Meier curve for overall survival showing RA-PSC-CM treatment is associated with reduced survival after 4 weeks (* p=0.04, Log-rank test).

FIG. 7. RA-PSC-CM grafts are enriched for atrial myocyte markers and show reduced sarcomeric protein and connexin 43 organization in comparison to PSC-CM grafts. (a) Representative immunofluorescence images from PSC-CM and RA-PSC-CM treated hearts showing PSC-CM grafts are composed almost entirely of MLC2v+ myocytes, in contrast to RA-PSC-CM grafts, which predominantly contain MLC2a+ myocytes. (b) Representative immunofluorescence images from PSC-CM and RA-PSC-CM treated hearts, showing increased abundance of arrhythmogenic CD172a+/CD200+ myocytes in RA-PSC-CM grafts. (c) Low magnification immunofluorescent images of cardiac troponin T (cTnT) and connexin 43 (Cx43) staining in PSC-CM and RA-PSC-CM grafts, showing increased expression of cTnT in PSC-CM grafts compared to RA-PSC-CM. In both graft types, Cx43 expression is less abundant than in the surrounding pig myocardium. (d) High magnification confocal images of the previous grafts showing highly organized and aligned sarcomeres with appropriate localization of Cx43 to the intercalated disks in PSC-CM graft. In stark contrast, RA-PSC-CM graft has disorganized cTnT expression, with sporadic expression of Cx43. (e) Immunofluorescent staining for CD31 and alpha smooth muscle actin shows an abundance of neovessels in both PSC-CM and RA-PSC-CM grafts. (f) Spatial transcriptomic analysis of PSC-CM and RA-PSC-CM grafts shows reduced expression of atrial natriuretic peptide (NPPA) and increased expression of MYL2/MLC2v in PSC-CM graft. This phenotype is reversed in RA-PSC-CM graft.

FIG. 8. Catheter ablation is a feasible and effective EA treatment strategy. (a) Representative rhythm strip showing termination of EA during CA. (b) Electroanatomic maps from representative CA treated subject. Top—Activation map of EA showing anatomic origin of arrhythmia (early activation—white, late activation—purple). Bottom—Activation map overlaid with ablation lesions (brown circles) which were delivered at sites of earliest activation resulting in termination of arrhythmia. (c) Plot depicting percentage of day spent in ventricular arrhythmia in subjects treated with catheter ablation. Two ablation procedures required for RA-PSC-CM subject and one procedure required for PSC-CM subject. (d) Anatomic correlation between endocardial activation maps and extracted heart in RA-PSC-CM subject. Left—Representative activation maps in which 4 unique EAs were encountered. EAs 1-3 were terminated during CAs 1 and 2 with final ablation lesions highlighted (brown circle with yellow outline). EA4 was persistent after CA2 and mapped at terminal EPS. Right—Apical view of extracted heart following euthanasia and formalin fixation. Ablation lesions (EAs 1-3) and island of surviving cell graft (EA4) outlined with excellent anatomic correlation to preceding activation maps. (e) Representative rhythm strips from RA-PSC-CM subject of sinus rhythm, EA 1, EA 2, and EA 4. (f) Plot depicting daily heart rate of EA 1, 2 and 4 in RA-PSC-CM subject (mean±SEM). Each new EA was of a significantly slower heart rate than the previously ablated EAs (EA 1 vs EA 2—** p=0.01, EA 1 vs EA 4—*** p=0.0003, EA 2 vs EA 4—* p=0.04; Ordinary one-way ANOVA with Sidak's multiple comparisons test). (g) Whole-mount cross-section of RA-PSC-CM+CA subject. Engrafted human cardiomyocytes expressed cardiac troponin T (red) and the human-specific anti-nuclear antigen Ku80 (brown). Scarred myocardium, either from percutaneous myocardial infarction or catheter ablation, was identified with aniline blue counterstaining. A small region of surviving human cardiomyocyte graft responsible for residual arrhythmias was identified (boxed region, zoomed on right).

FIG. 9. Characterisation of PSC-CM derived from DEAB treated cultures. (a) Scheme of small molecule directed differentiation with DEAB used for cardiomyocyte differentiation from hPSCs SCVI8. RT-PCR analysis of ventricular-associated genes (b)-(e) and atrial/pacemaker associated genes (f)-(g) at Day 14. Values represent expression levels relative to housekeeping gene 18S. *p<0.05, **p<0.01, determined by one-way ANOVA and Dunnett's post hoc-testing from n=3 independent experiments

### DEFINITIONS

As used in this application, the singular form “a”, “an” and “the” include plural references unless the context clearly dictates otherwise.

As used herein, the term “comprising” means “including.” Variations of the word “comprising”, such as “comprise” and “comprises,” have correspondingly varied meanings.

It will be understood that use of the term “about” herein in reference to a recited numerical value includes the recited numerical value and numerical values within plus or minus ten percent of the recited value.

It will be understood that use of the term “between” herein when referring to a range of numerical values encompasses the numerical values at each endpoint of the range. For example, a concentration of between 2 mg/mL and 10 mg/mL is inclusive of a concentration of 2 mg/mL and a concentration of 10 mg/mL.

The terms ‘subject’ and ‘patient’ may be used interchangeably and refer to a human or other mammal suffering from, at risk of suffering from, arrhythmia following transplantation of PSC-CMs.

Whether a marker, such as CD200, CD90, or CD172a, is or is not expressed on a surface of a cell, such as a PSC-CM, may be signified by the designation +/−. For example, stating that a cell is or has the signature CD90+ is equivalent to stating that the cell expresses CD90 on a surface thereof.

The surface marker CD172a refers to signal regulatory protein α (SIRPα). In the context of this specification, CD172a may also be referred to as SIRPA. In the context of this specification, the terms CD172a, SIRPα and SIRPA are used interchangeably.

In the context of this specification ‘arrhythmia’ refers to ventricular arrhythmia, and may refer specifically to ventricular tachycardia. Ventricular tachycardia may be defined as a broad complex rhythm with QRS duration >120 ms (as measured by electrocardiography) and heart rate >100 beats per minute.

In the context of this specification, an ‘arrhythmogenic’ cell or (sub)population of cells is understood to be a cell or (sub)population of cells which causes a particularly high incidence of arrhythmia upon transplantation in a subject. Particularly, it is a cell or (sub)population of cells which causes arrhythmia even when mature (i.e. after a period of time post-transplantation). All PSC-CMs initially exhibit automaticity and may cause arrhythmia, as they are immature compared to adult myocytes and do not immediately electrically couple with the heart. However, certain subpopulations of cells, referred to as arrhythmogenic cells in this specification, are believed not lose arrhythmogenic properties despite maturation of the overall PSC-CM graft. That is, an arrhythmogenic cell may cause arrhythmias despite a period of maturation in vivo. The term ‘non-arrhythmogenic’ has a correspondingly opposite meaning. In the context of this specification, a non-arrhythmogenic cell or (sub)population of cells does not cause a high incidence of arrhythmia upon transplantation in a subject. Particularly, a non-arrhythmogenic cell or (sub)population of cells does not cause arrhythmia after a period of maturation in vivo. A period of maturation may refer to a period of several weeks, such as 2 weeks, or 3, 4, 5, 6, 7, 8, 9, 10, 11, or 12 weeks.

A ‘purified’ population of cells refers to a population of cells that is enriched for a selected cell type. For example, a ‘purified population of non-arrhythmogenic PSC-CMs’ refers to a population of PSC-CMs that is enriched for non-arrhythmogenic PSC-CMs. The purified population can be at least 60%, 65%, 70%, 75%, 80%, 85%, 90%, 95%, 96%, 97%, 98%, 99%, or 100% enriched for the desired cell type.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### DESCRIPTION OF EMBODIMENTS

Current cardiac differentiation protocols yield heterogeneous cellular outputs. Although ventricular cardiomyocytes predominate, other cell types such as atrial and pacemaker-like cells along with non-myocytes are also present. To date, no study had robustly assessed how the composition of PSC-CM cell doses may impact EA burden. The present inventors have surprisingly found that presence of PSC-CMs having the surface marker signature CD172a+/CD90−/CD200+ in a cell dose is positively correlated with the incidence of arrhythmia upon transplantation in a subject. Presence of PSC-CMs having the surface marker signature CD172a+/CD90−/CD200− in a cell dose was found to be negatively correlated with incidence of arrhythmia upon transplantation in a subject.

As the CD172a+/CD90−/CD200+ signature can be readily identified by well-established methods such as flow cytometry, the methods of the invention provide a rapid, simple, and convenient means to identify the presence of arrhythmogenic cells, and by extension, to remove arrhythmogenic cells from cell doses, reducing arrhythmia upon transplantation.

### 1. Identification of Arrhythmogenic PSC-CMs

In a first aspect of the invention, there is provided a method of identifying an arrhythmogenic pluripotent stem cell-derived cardiomyocyte (PSC-CM).

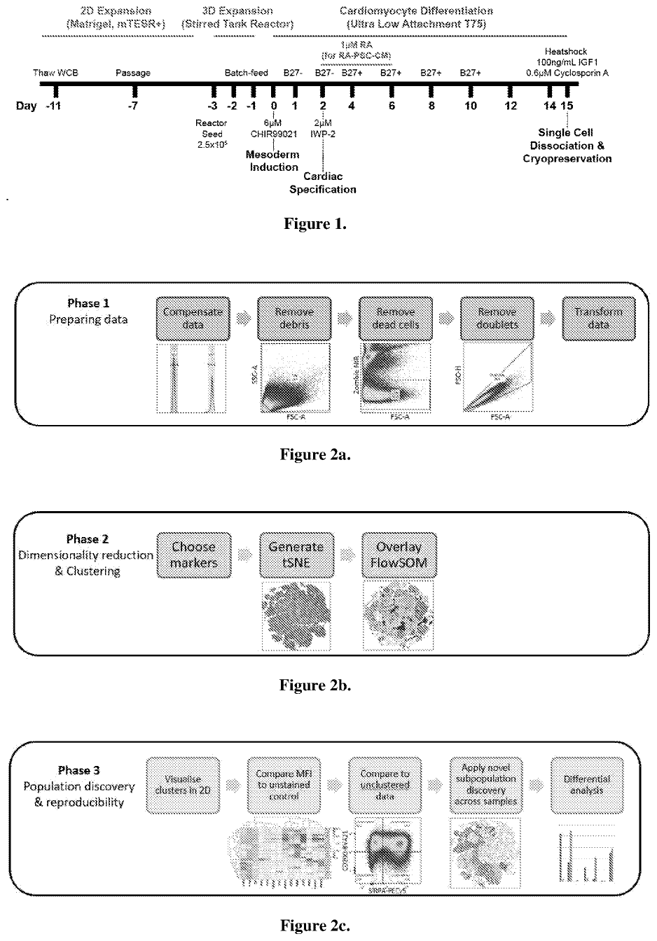

A pluripotent stem cell-derived cardiomyocyte refers to a cardiomyocyte which has been obtained by causing a pluripotent stem cell to differentiate and develop into a cardiomyocyte. Generally speaking, this is involves mimicking in vivo development by altering signaling pathways and the cells' microenvironment. PSC-CM are typically produced in a series of stages from pluripotent stem cells (PSCs): 2D expansion, 3D expansion, then followed by cardiomyocyte differentiation (which involves mesoderm induction, followed by cardiac specification). See FIG. 1. This forms a heterogeneous mixture of pluripotent stem cell-derived cardiac cells, some of which may be PSC-CMs. PSC-CMs are themselves also heterogenous. The PSC-CM's may be produced by any suitable protocol known to the skilled person such as for example that of Chen et al. (Stem Cell Res 2015, 15(2), p. 365). The PSC may be a human PSC, resulting in formation of a human pluripotent stem cell-derived cardiomyocyte (hPSC-CM), or it may be a non-human PSC, resulting in the formation of a non-human pluripotent stem cell-derived cardiomyocyte. The PSC may be derived from any mammal. The term human pluripotent stem cell-derived cardiomyocyte (PSC-CM) is understood to encompass both human and non-human derived cells.

An arrhythmogenic PSC-CM is a PSC-CM which, upon transplantation into a subject, causes a particularly high incidence of arrhythmia upon transplantation in a subject, as defined above. Particularly, it is a cell or (sub)population of cells which causes arrhythmia even when mature.

The method of the first aspect of the invention comprises determining whether CD200 is expressed on a surface of the PSC-CM. If CD200 is expressed on a surface of the PSC-CM, the PSC-CM is an arrhythmogenic PSC-CM. If CD200 is not expressed on the surface of the PSC-CM, the PSC-CM is not an arrhythmogenic PSC-CM.

The method of the first aspect of the invention may also comprise determining whether other makers are expressed on (or absent from) the surface of the PSC-CM. In one embodiment, the method comprises determining whether CD200 is expressed on a surface of the PSC-CM, as well as determining whether CD172a and/or CD90 are expressed on the surface of the PSC-CM. In an embodiment, the method may comprise determining whether CD200 and CD172a are expressed on the surface of the PSC-CM. In this case, if CD200 and CD172a are both expressed on a surface of the PSC-CM, the PSC-CM is an arrhythmogenic PSC-CM. Conversely, if CD200 is not expressed on the surface of the PSC-CM and CD172a is expressed on the surface of the PSC-CM, the CM is not an arrhythmogenic PSC-CM. In an embodiment, the method may comprise determining whether CD200 and CD90 are expressed on the surface of the PSC-CM. In this case, if CD200 is expressed on a surface of the PSC-CM, and CD90 is not expressed on the surface of the PSC-CM, the PSC-CM is an arrhythmogenic PSC-CM. Conversely, if neither CD200 nor CD90 are expressed on the surface of the PSC-CM, the PSC-CM is not an arrhythmogenic PSC-CM. In another embodiment, the method comprises determining whether CD200, CD172a, and CD90 are expressed on a surface of the PSC-CM. In this case, if CD200 and CD172a are expressed on the surface of the PSC-CM, and CD90 is not expressed on the surface of the PSC-CM, the PSC-CM is an arrhythmogenic PSC-CM. Conversely, if CD200 and CD90 are not expressed on the surface of the PSC-CM, and CD172a is expressed on the surface of the PSC-CM, the PSC-CM is not an arrhythmogenic PSC-CM.

The step of determining whether CD200, and optionally other markers, are expressed on a surface of the PSC-CM may be carried out using any suitable method known to the skilled person. In one embodiment, the PSC-CM may be exposed to an agent containing a detectable label, wherein the agent selectively binds to the marker. Excess agent containing a detectable label is then removed, e.g. by washing. If the marker is present on the surface of the PSC-CM, the agent binds to the marker, providing a labelled PSC-CM. Thus a labelled PSC-CM is a PSC-CM which expresses the marker (such as CD200, or whichever marker the agent selectively binds to). The detectable label can then be detected, confirming the presence of the marker. In one embodiment, the marker is CD200 and the agent containing a detectable label selectively binds to CD200. In one embodiment, the marker is CD90 and the agent containing a detectable label selectively binds to CD90. In one embodiment, the marker is CD172a and the agent containing a detectable label selectively binds to CD172a.

If the expression of multiple markers is to be determined, the PSC-CM may be treated with multiple agents containing detectable labels, each of which is selective for one of the markers to be determined, and each bearing a distinct detectable label. In one embodiment, in order to determine whether CD200, CD172a and CD90 are expressed on the surface of the PSC-CM, the PSC-CM is exposed to a first agent comprising a first detectable label that selectively binds to CD200, a second agent comprising a second detectable label that selectively binds to CD172a, and a third agent comprising a third detectable label that selectively binds to CD90, to provide a triple labelled PSC-CM. Excess agent containing a detectable label is then removed, e.g. by washing, followed by detecting each of the detectable labels.

In one embodiment, the agent which selectively binds to a marker may be an antibody, and the detectable label may be a fluorophore. In this case, the expression of a marker is indicated by the wavelength and intensity of fluorescence emitted from the labelled PSC-CM. In some embodiments, the antibody may be an anti-CD200, anti-CD172a or anti-CD90 antibody, which is conjugated to a fluorophore. Many anti-CD200, anti-CD172a, and anti-CD90 antibodies, which are conjugated to a fluorophore, are known to the skilled person and available commercially, any of which is suitable to use in the methods of the invention. Examples of suitable antibodies conjugated to fluorophores include anti-CD172a PECy5, anti-CD90 BV650, and anti-CD200 BV421.

In another embodiment, the agent which selectively binds to a marker may be an antibody, and the detectable label may be a magnetic bead. In this case, the expression of a marker is indicated by the response of the PSC-CM to a magnetic field. In some embodiments, the antibody may be an anti-CD200, anti-CD172a or anti-CD90 antibody, which is conjugated to a magnetic bead. Many anti-CD200, anti-CD172a, and anti-CD90 antibodies, which are conjugated to magnetic beads, are known to the skilled person and available commercially, any of which is suitable to use in the methods of the invention.

In one embodiment, determining whether CD200, and optionally other markers, are expressed on a surface of the PSC-CM may be carried out by genetic modification to incorporate a reporter gene in the PSC-CM, wherein the reporter gene is transcribed in conjunction with transcription of the gene for the marker, and the reporter gene encodes a detectable product. Thus the detectable product is produced when the gene for the marker is transcribed. For example, the reporter gene may encode green fluorescent protein (GFP) or luciferase. If the expression of multiple markers is to be determined, multiple distinct reporter genes may be required. Genetic modification techniques for introducing a reporter gene into a cell line are known to the skilled person, and may be carried out using commercially available kits. Any such technique is suitable for use with the methods of the invention.

In one embodiment, the step of determining whether CD200, and optionally other markers, are expressed on a surface of the PSC-CM is carried out using flow cytometry. Typically in this case, the agent which selectively binds to a marker is an antibody, and the detectable label is a fluorophore. Preliminary processing steps may be undertaken prior to determining whether CD200, and optionally other markers, are expressed on a surface of the PSC-CM using flow cytometry. This may include compensation, staining with a viability marker (for example Zombie NIR), and manual gating to remove artefacts including debris, dead cells and doublets.

Flow cytometry enables the detection of labelled PSC-CMs amongst a plurality of PSC-CMs. Typically, appropriate fluorescence intensity thresholds (‘gates’) are set by the operator for a control sample, wherein if fluorescence levels of a cell exceed these gate levels, the cell is counted as a labelled PSC-CM. The fluorescence intensity values for the gates are dependent on the antibody label and will differ from one flow cytometer to another, as well as between individual runs and between different batches of labelled antibodies. A commonly used guideline is to set the gates such that 0.5% or less of events for the control sample fall outside each gate. These gates are then applied to the sample to be measured, i.e. the sample containing PSC-CMs that have been exposed to an antibody comprising a detectable label which is a fluorophore.

### 2. Screening for Arrhythmogenic PSC-CMs

In a second aspect of the invention, there is provided a method of determining whether a dose containing a plurality of PSC-CMs is likely to cause arrhythmia upon transplantation in a subject.

Transplantation of a dose of PSC-CM is a potential treatment for replacement of damaged or destroyed cardiomyocytes following myocardial infarction. According to the second aspect of the invention, such a dose may be screened for the presence of arrhythmogenic PSC-CM's prior to transplantation in order to assess whether it is likely to cause engraftment arrhythmia.

A dose of PSC-CM refers to a plurality of PSC-CM, which are as described above in Section 1. A dose typically refers to an amount of PSC-CM intended for transplantation into a subject.

Transplantation of the dose may involve, for example, injection of the dose into the heart of a subject, specifically into infarct and border zones.

In the method of the second aspect of the invention, the dose is subjected to the method of the first aspect of the invention as described in Section 1, to identify whether the dose contains arrhythmogenic PSC-CM. That is, it is determined whether a dose containing a plurality of PSC-CMs contains PSC-CMs which express CD200 (and optionally other markers such as CD172a and/or CD90) on a surface thereof, wherein a PSC-CM which expresses CD200 on a surface thereof (and optionally expresses CD172a and/or does not express CD90) is identified as an arrhythmogenic PSC-CM. It is understood that the method of the first aspect of the invention may be applied to a representative sample taken from the dose to determine whether CD200 (and optionally other markers such as CD172a and/or CD90) is expressed on a surface of the plurality of PSC-CMs contained in the dose. If the dose contains arrhythmogenic PSC-CM, then the dose is likely to cause arrhythmia upon transplantation to a subject.

The method of the second aspect of the invention may further comprise determining the proportion of arrhythmogenic PSC-CM in the dose, i.e. the percentage of the plurality of PSC-CM contained in the dose which are arrhythmogenic PSC-CM. If the proportion of arrhythmogenic PSC-CM in the dose is greater than about 0.001%, the dose is likely to cause arrhythmia upon transplantation in the subject. If the proportion of arrhythmogenic PSC-CM in the dose is greater than about 0.005%, or about 0.01, 0.05, 0.1, 0.5, 1, 2, 2.5, 5, 10, 15, or 20%, the dose is likely to cause arrhythmia upon transplantation in the subject.

### 3. Cell Sorting for Arrhythmogenic PSC-CMs

In a third aspect of the invention, there is provided a method of eliminating or reducing arrhythmia following transplantation of a dose containing a plurality of PSC-CMs in a subject.

Transplantation of a dose of PSC-CM is a potential treatment for replacement of damaged or destroyed cardiomyocytes following myocardial infarction. The third aspect of the invention provides a method to remove arrhythmogenic PSC-CM from the dose prior to transplantation, in order to eliminate or reduce arrhythmia.

The method of the third aspect of the invention is carried out prior to the transplantation of a dose of PSC-CMs in a subject. Firstly, a crude dose of PSC-CM is subjected to the method of the first aspect of the invention as described in Section 1, to identify arrhythmogenic PSC-CM. That is, it is determined whether any of the plurality of the PSC-CM contained in the dose express CD200 (and optionally other markers such as CD172a and/or CD90) on a surface thereof, wherein a PSC-CM which expresses CD200 on a surface thereof (and optionally expresses CD172a and/or does not express CD90) is identified as an arrhythmogenic PSC-CM.

A crude dose of PSC-CM means a dose comprising a plurality of pluripotent stem cells (PSCs) which have been differentiated to form PSC-CMs, but have not been subjected to any additional treatments intended to enrich or deplete a subpopulation of cardiomyocytes. A crude dose is also understood to be a dose which has not been subjected to the method of the third aspect of the invention.

Secondly, in the method of the third aspect of the invention, the arrhythmogenic PSC-CM are removed from the crude dose to provide a purified dose. The arrhythmogenic PSC-CM may be removed from the crude dose using any suitable method known to the skilled person. In one embodiment, the arrhythmogenic PSC-CM are removed from the crude dose using fluorescence-activated cell sorting (FACS). In this case, in the method of identifying an arrhythmogenic PSC-CM, the PSC-CM is exposed to an agent containing a detectable label, wherein the agent selectively binds to the marker, which is an antibody conjugated to a fluorophore. Typically, in a FACS system, individual cells are placed into droplets and, depending on the wavelength and intensity of fluorescence emitted from the cell, an electrical charge is applied to the droplet so that it can be deflected by an electromagnetic field for separate collection as required. In one embodiment, the arrhythmogenic PSC-CM are removed from the crude dose using magnetic-activated cell sorting (MACS). In this case, in the method of identifying an arrhythmogenic PSC-CM, the PSC-CM is exposed to an agent containing a detectable label, wherein the agent selectively binds to the marker, which is an antibody conjugated to a magnetic bead. Typically, in a MACS system, cells are passed through a magnetized column. Those cells which are labelled with a magnetic bead are magnetized to the walls of the column, and unlabeled cells pass through the column, and are thus separated. The steps of identifying arrhythmogenic PSC-CM and removing arrhythmogenic PSC-CM may be performed sequentially, for example as occurs in a FACS system, or simultaneously, for example as occurs in a MACS system.

Following transplantation of the purified dose in a subject, arrhythmia may be eliminated or reduced compared to arrhythmia following transplantation of the crude dose. This may be assessed in practice by comparing arrhythmia experienced by a test subject who received the purified dose, to arrhythmia experienced by a control subject who received the crude dose. Arrhythmia may be reduced as defined by the cumulative time per day spent in arrhythmia over 25 days post-transplantation. Arrhythmia may be reduced by at least about 10%, or at least about 20, 30, 40, 50, 60, 70, 75, 80, 85, 90, 95, 99 or at least about 100%. Arrhythmia may be eliminated.

Step (ii) of the method of the third aspect of the invention may comprise removing at least about 50% of the arrhythmogenic PSC-CMs from the crude dose. It may comprise removing at least about 60%, or at least about 70, 80, 90, 95, or 100% of the arrhythmogenic PSC-CMs from the crude dose.

In a fourth aspect of the invention, there is provided a method of providing a dose containing a plurality of PSC-CM which is substantially free from arrhythmogenic PSC-CMs.

In the method of the fourth aspect of the invention, firstly, a crude dose of PSC-CM is subjected to the method of the first aspect of the invention as described in Section 1, to identify arrhythmogenic PSC-CM. That is, it is determined whether any of the plurality of the PSC-CM contained in the dose express CD200 (and optionally other markers such as CD172a and/or CD90) on a surface thereof, wherein a PSC-CM which expresses CD200 on a surface thereof (and optionally expresses CD172a and/or does not express CD90) is identified as an arrhythmogenic PSC-CM. A crude dose is as described above in respect of the third aspect of the invention.

Secondly, in the method of the fourth aspect of the invention, the arrhythmogenic PSC-CM are removed from the crude dose to provide a dose of PSC-CM which is substantially free from arrhythmogenic PSC-CMs. Step (ii) may be performed by any suitable method known to the skilled person, as described above for the corresponding step of the method of the third aspect of the invention.

Step (ii) of the method of the fourth aspect of the invention may comprise removing at least about 50% of the arrhythmogenic PSC-CMs from the crude dose. It may comprise removing at least about 60%, or at least about 70, 80, 90, 95, or 100% of the arrhythmogenic PSC-CMs from the crude dose.

The dose of PSC-CM which is substantially free from arrhythmogenic PSC-CMs may contain less than 0.001% of arrhythmogenic PSC-CMs, relative to the total number of PSC-CM in the dose. The dose of PSC-CM which is substantially free from arrhythmogenic PSC-CMs may contain less than 0.005%, or less than about 0.01, 0.05, 0.1, 0.5, 1, 2, 2.5, 5, 10, 15, or 20% of arrhythmogenic PSC-CMs, relative to the total number of PSC-CM in the dose.

### 4. Identification of Pacemaker Cells and Providing a Population of Pacemaker Cells

In a fifth aspect of the invention, there is provided a method of identifying a pluripotent stem cell-derived cardiac cell with pacemaker properties (PSC-PM).

A pure or substantially pure population of pacemaker cells may provide a useful substrate for biological research and for drug testing. For example, a population of pacemaker cells may be useful in an assay for testing drugs for cardiac side-effects.

A pluripotent stem cell-derived cardiac cell with pacemaker properties (PSC-PM) refers to a cell which has been obtained from pluripotent stem cells that have been caused to differentiate and develop into cardiomyocytes. Generally speaking, this is involves mimicking in vivo development by altering signaling pathways and the cells' microenvironment. Pluripotent stem cell-derived cardiac cells are typically produced in a series of stages from pluripotent stem cells (PSCs): 2D expansion, 3D expansion, then followed by cardiomyocyte differentiation (which involves mesoderm induction, followed by cardiac specification). See FIG. 1. This forms a heterogeneous mixture of pluripotent stem cell-derived cardiac cells, some of which may be PSC-PMs. The PSC-PMs may be produced by any suitable protocol known to the skilled person such as for example that of Chen et al. (Stem Cell Res 2015, 15(2), p. 365). The PSC may be a human PSC, resulting in formation of a human pluripotent stem cell-derived cardiac cells, or it may be a non-human PSC, resulting in the formation of a non-human pluripotent stem cell-derived cardiac cells. The PSC may be derived from any mammal. The term pluripotent stem cell-derived cardiac cells and PSC-PM is understood to encompass both human and non-human derived cells.

The method of the fifth aspect of the invention comprises determining whether CD200 is expressed on a surface of a pluripotent stem cell-derived cardiac cell. If CD200 is expressed on a surface of the pluripotent stem cell-derived cardiac cell, the pluripotent stem cell-derived cardiac cell is an PSC-PM. If CD200 is not expressed on the surface of the pluripotent stem cell-derived cardiac cell, the pluripotent stem cell-derived cardiac cell is not a PSC-PM.

The method of the fifth aspect of the invention may also comprise determining whether other makers are expressed on (or absent from) the surface of the pluripotent stem cell-derived cardiac cell. In one embodiment, the method comprises determining whether CD200 is expressed on a surface of the pluripotent stem cell-derived cardiac cell, as well as determining whether CD172a and/or CD90 are expressed on the surface of the pluripotent stem cell-derived cardiac cell. In an embodiment, the method may comprise determining whether CD200 and CD172a are expressed on the surface of the pluripotent stem cell-derived cardiac cell. In this case, if CD200 and CD172a are both expressed on a surface of the pluripotent stem cell-derived cardiac cell, the cell is a PSC-PM. Conversely, if CD200 is not expressed on the surface of the pluripotent stem cell-derived cardiac cell and CD172a is expressed on the surface of the pluripotent stem cell-derived cardiac cell, the cell is not a PSC-PM. In an embodiment, the method may comprise determining whether CD200 and CD90 are expressed on the surface of the pluripotent stem cell-derived cardiac cell. In this case, if CD200 is expressed on a surface of the pluripotent stem cell-derived cardiac cell, and CD90 is not expressed on the surface of the pluripotent stem cell-derived cardiac cell, the cell is a PSC-PM. Conversely, if neither CD200 nor CD90 are expressed on the surface of the pluripotent stem cell-derived cardiac cell, the cell is not an PSC-PM. In another embodiment, the method comprises determining whether CD200, CD172a, and CD90 are expressed on a surface of the pluripotent stem cell-derived cardiac cell. In this case, if CD200 and CD172a are expressed on the surface of the pluripotent stem cell-derived cardiac cell, and CD90 is not expressed on the surface of the pluripotent stem cell-derived cardiac cell, the cell is a PSC-PM. Conversely, if CD200 and CD90 are not expressed on the surface of the pluripotent stem cell-derived cardiac cell, and CD172a is expressed on the surface of the pluripotent stem cell-derived cardiac cell, the cell is not a PSC-PM.

The step of determining whether CD200, and optionally other markers, are expressed on a surface of the pluripotent stem cell-derived cardiac cell may be carried out using any suitable method known to the skilled person. In one embodiment, the pluripotent stem cell-derived cardiac cell may be exposed to an agent containing a detectable label, wherein the agent selectively binds to the marker. Excess agent containing a detectable label is then removed, e.g. by washing. If the marker is present on the surface of the pluripotent stem cell-derived cardiac cell, the agent binds to the marker, providing a labelled pluripotent stem cell-derived cardiac cell. Thus a labelled pluripotent stem cell-derived cardiac cell is a cell which expresses the marker (such as CD200, or whichever marker the agent selectively binds to). The detectable label can then be detected, confirming presence of the marker. In one embodiment, the marker is CD200 and the agent containing a detectable label selectively binds to CD200. In one embodiment, the marker is CD90 and the agent containing a detectable label selectively binds to CD90. In one embodiment, the marker is CD172a and the agent containing a detectable label selectively binds to CD172a.

If the expression of multiple markers is to be determined, the pluripotent stem cell-derived cardiac cell may be treated with multiple agents containing detectable labels, each of which is selective for one of the markers to be determined, and each bearing a distinct detectable label. In one embodiment, in order to determine whether CD200, CD172a and CD90 are expressed on the surface of the pluripotent stem cell-derived cardiac cell, the pluripotent stem cell-derived cardiac cell is exposed to a first agent comprising a first detectable label that selectively binds to CD200, a second agent comprising a second detectable label that selectively binds to CD172a, and a third agent comprising a third detectable label that selectively binds to CD90, to provide a triple labelled pluripotent stem cell-derived cardiac cell. Excess agent containing a detectable label is then removed, e.g. by washing, followed by detecting each of the detectable labels.

In one embodiment, the agent which selectively binds to a marker may be an antibody, and the detectable label may be a fluorophore. In this case, the expression of a marker is indicated by the wavelength and intensity of fluorescence emitted from the labelled pluripotent stem cell-derived cardiac cell. In some embodiments, the antibody may be an anti-CD200, anti-CD172a or anti-CD90 antibody, which is conjugated to a fluorophore. Many anti-CD200, anti-CD172a, and anti-CD90 antibodies, which are conjugated to a fluorophore, are known to the skilled person and available commercially, any of which is suitable to use in the methods of the invention. Examples of suitable antibodies conjugated to fluorophores include anti-CD172a PECy5, anti-CD90 BV650, and anti-CD200 BV421.

In another embodiment, the agent which selectively binds to a marker may be an antibody, and the detectable label may be a magnetic bead. In this case, the expression of a marker is indicated by the response of the pluripotent stem cell-derived cardiac cell to a magnetic field. In some embodiments, the antibody may be an anti-CD200, anti-CD172a or anti-CD90 antibody, which is conjugated to a magnetic bead. Many anti-CD200, anti-CD172a, and anti-CD90 antibodies, which are conjugated to magnetic beads, are known to the skilled person and available commercially, any of which is suitable to use in the methods of the invention.

In one embodiment, determining whether CD200, and optionally other markers, are expressed on a surface of the pluripotent stem cell-derived cardiac cell may be carried out by genetic modification to incorporate a reporter gene in the pluripotent stem cell-derived cardiac cell, wherein the reporter gene is transcribed in conjunction with transcription of the gene for the marker, and the reporter gene encodes a detectable product. Thus the detectable product is produced when the gene for the marker is transcribed. For example, the reporter gene may encode green fluorescent protein (GFP) or luciferase. If the expression of multiple markers is to be determined, multiple distinct reporter genes may be required. Genetic modification techniques for introducing a reporter gene into a cell line are known to the skilled person, and may be carried out using commercially available kits. Any such technique is suitable for use with the methods of the invention.

In one embodiment, the step of determining whether CD200, and optionally other markers, are expressed on a surface of the pluripotent stem cell-derived cardiac cell is carried out using flow cytometry. Typically in this case, the agent which selectively binds to a marker is an antibody, and the detectable label is a fluorophore. Preliminary processing steps may be undertaken prior to determining whether CD200, and optionally other markers, are expressed on a surface of the pluripotent stem cell-derived cardiac cell. This may include compensation, staining with a viability marker (for example Zombie NIR), and manual gating to remove artefacts including debris, dead cells and doublets.

Flow cytometry enables the detection of labelled pluripotent stem cell-derived cardiac cells amongst a large number of cells. Typically, appropriate fluorescence intensity thresholds (‘gates’) are set by the operator for a control sample, wherein if fluorescence levels of a cell exceed these gate levels, the cell is counted as a labelled pluripotent stem cell-derived cardiac cell. The fluorescence intensity values for the gates are dependent on the antibody label and will differ from one flow cytometer to another, as well as between individual runs and between different batches of antibodies. A commonly used guideline is to set the gates such that 0.5% or less of events for the control sample fall outside each gate. These gates are then applied to the sample to be measured, i.e. the sample containing pluripotent stem cell-derived cardiac cells that have been exposed to an antibody comprising a detectable label which is a fluorophore.

In a sixth aspect of the invention, there is provided a method of providing a substantially pure dose of pluripotent stem cell-derived cardiac cells with pacemaker properties (PSC-PMs).

In the method of the sixth aspect of the invention, a plurality of pluripotent stem cell-derived cardiac cells are subjected to the method of the fifth aspect of the invention as described above, to identify the PSC-PMs. That is, it is determined which of the pluripotent stem cell-derived cardiac cells express CD200 (and optionally other markers such as CD172a and/or CD90) on a surface thereof, wherein a pluripotent stem cell-derived cardiac cells which expresses CD200 on a surface thereof (and optionally expresses CD172a and/or does not express CD90) is identified as a PSC-PM.

Secondly, in the method of the sixth aspect of the invention, the PSC-PMs are isolated from the plurality of pluripotent stem cell-derived cardiac cells to provide a substantially pure dose of PSC-PMs. The PSC-PMs may be isolated from the plurality of pluripotent stem cell-derived cardiac cells using any suitable method known to the skilled person. In one embodiment, the PSC-PM are isolated from the plurality of pluripotent stem cell-derived cardiac cells using fluorescence-activated cell sorting (FACS). In this case, in the method of identifying a PSC-PM, the pluripotent stem cell-derived cardiac cells are exposed to an agent containing a detectable label, wherein the agent selectively binds to the marker, which is an antibody conjugated to a fluorophore. Typically, in a FACS system, individual cells are placed into droplets and, depending on the wavelength and intensity of fluorescence emitted from the cell, an electrical charge is applied to the droplet so that it can be deflected by an electromagnetic field for separate collection as required. In one embodiment, the PSC-PM are isolated from the plurality of pluripotent stem cell-derived cardiac cells using magnetic-activated cell sorting (MACS). In this case, in the method of identifying a PSC-PM, the plurality of pluripotent stem cell-derived cardiac cells are exposed to an agent containing a detectable label, wherein the agent selectively binds to the marker, which is an antibody conjugated to a magnetic bead. Typically, in a MACS system, cells are passed through a magnetized column. Those cells which are labelled with a magnetic bead are magnetized to the walls of the column, and unlabeled cells pass through the column, and are thus separated. The steps of identifying PSC-PMs and isolating PSC-PMs may be performed sequentially, for example as occurs in a FACS system, or simultaneously, for example as occurs in a MACS system.

The method of the sixth aspect of the invention provides a dose of PSC-PM which is substantially pure. Substantially pure means that no more than 0.001% of cells in the dose are not PSC-PM, relative to the total number of cells in the dose, or that no more than about 0.005%, or than about 0.01, 0.05, 0.1, 0.5, 1, 2, 2.5, 5, 10, 15, or 20% of cells in the dose are not PSC-PM. Whether a cell is or is not a PSC-PM is determined as described above in respect of the fifth aspect of the invention.

### 5. Identification of Ventricular Cells and Providing a Population of Ventricular Cells

In a seventh aspect of the invention, there is provided a method of identifying a pluripotent stem cell-derived ventricular cardiomyocyte (PSC-VM).

A pure or substantially pure population of ventricular cardiomyocytes may provide a useful substrate for biological research and for drug testing. For example, a pure population of ventricular cardiomyocytes may be useful in an assay for testing drugs for cardiac side-effects.

A pluripotent stem cell-derived ventricular cardiomyocyte (PSC-VM) refers to a cell which has been obtained from pluripotent stem cells that have been caused to differentiate and develop into cardiomyocytes. Generally speaking, this is involves mimicking in vivo development by altering signaling pathways and the cells' microenvironment. Pluripotent stem cell-derived cardiac cells are typically produced in a series of stages from pluripotent stem cells (PSCs): 2D expansion, 3D expansion, then followed by cardiomyocyte differentiation (which involves mesoderm induction, followed by cardiac specification). See FIG. 1. This forms a heterogeneous mixture of pluripotent stem cell-derived cardiac cells, some of which may be PSC-VMs. The PSC-VMs may be produced by any suitable protocol known to the skilled person such as for example that of Chen et al. (Stem Cell Res 2015, 15(2), p. 365). The PSC may be a human PSC, resulting in formation of a human pluripotent stem cell-derived cardiac cells, or it may be a non-human PSC, resulting in the formation of a non-human pluripotent stem cell-derived cardiac cells. The PSC may be derived from any mammal. The term pluripotent stem cell-derived cardiac cells and PSC-VM is understood to encompass both human and non-human derived cells.

The method of the seventh aspect of the invention comprises determining whether CD200 is expressed on a surface of a pluripotent stem cell-derived cardiac cell. If CD200 is not expressed on a surface of the pluripotent stem cell-derived cardiac cell, the pluripotent stem cell-derived cardiac cell is a PSC-VM. If CD200 is expressed on the surface of the pluripotent stem cell-derived cardiac cell, the pluripotent stem cell-derived cardiac cell is not a PSC-VM.

The method of the seventh aspect of the invention may also comprise determining whether other makers are expressed on (or absent from) the surface of the pluripotent stem cell-derived cardiac cell. In one embodiment, the method comprises determining whether CD200 is expressed on a surface of the pluripotent stem cell-derived cardiac cell, as well as determining whether CD172a and/or CD90 are expressed on the surface of the pluripotent stem cell-derived cardiac cell. In an embodiment, the method may comprise determining whether CD200 and CD172a are expressed on the surface of the pluripotent stem cell-derived cardiac cell. In this case, if CD200 is not expressed on a surface of the pluripotent stem cell-derived cardiac cell and CD172a is expressed on the surface of the pluripotent stem cell-derived cardiac cell, the cell is a PSC-VM. Conversely, if CD200 and CD172a are both expressed on the surface of the pluripotent stem cell-derived cardiac cell, the cell is not a PSC-VM. In an embodiment, the method may comprise determining whether CD200 and CD90 are expressed on the surface of the pluripotent stem cell-derived cardiac cell. In this case, if CD200 and CD90 are both not expressed on a surface of the pluripotent stem cell-derived cardiac cell, the cell is a PSC-VM. Conversely, if CD200 is expressed on a surface of the pluripotent stem cell-derived cardiac cell and CD90 is not expressed on the surface of the pluripotent stem cell-derived cardiac cell, the cell is not an PSC-VM. In another embodiment, the method comprises determining whether CD200, CD172a, and CD90 are expressed on a surface of the pluripotent stem cell-derived cardiac cell. In this case, if CD200 and CD90 are not expressed on a surface of the pluripotent stem cell-derived cardiac cell and CD172a is expressed on the surface of the pluripotent stem cell-derived cardiac cell, the cell is a PSC-VM. Conversely, if CD200 and CD172a are expressed on a surface of the pluripotent stem cell-derived cardiac cell, and CD90 is not expressed on the surface of the pluripotent stem cell-derived cardiac cell, the cell is not a PSC-VM.

The step of determining whether CD200, and optionally other markers, are expressed on a surface of the pluripotent stem cell-derived cardiac cell may be carried out using any suitable method known to the skilled person. In one embodiment, the pluripotent stem cell-derived cardiac cell may be exposed to an agent containing a detectable label, wherein the agent selectively binds to the marker. Excess agent containing a detectable label is then removed, e.g. by washing. If the marker is present on the surface of the pluripotent stem cell-derived cardiac cell, the agent binds to the marker, providing a labelled pluripotent stem cell-derived cardiac cell. Thus a labelled pluripotent stem cell-derived cardiac cell is a cell which expresses the marker (such as CD200, or whichever marker the agent selectively binds to). The detectable label can then be detected, confirming presence of the marker. In one embodiment, the marker is CD200 and the agent containing a detectable label selectively binds to CD200. In one embodiment, the marker is CD90 and the agent containing a detectable label selectively binds to CD90. In one embodiment, the marker is CD172a and the agent containing a detectable label selectively binds to CD172a.

If the expression of multiple markers is to be determined, the pluripotent stem cell-derived cardiac cell may be treated with multiple agents containing detectable labels, each of which is selective for one of the markers to be determined, and each bearing a distinct detectable label. In one embodiment, in order to determine whether CD200, CD172a and CD90 are expressed on the surface of the pluripotent stem cell-derived cardiac cell, the pluripotent stem cell-derived cardiac cell is exposed to a first agent comprising a first detectable label that selectively binds to CD200, a second agent comprising a second detectable label that selectively binds to CD172a, and a third agent comprising a third detectable label that selectively binds to CD90, to provide a triple labelled pluripotent stem cell-derived cardiac cell. Excess agent containing a detectable label is then removed, e.g. by washing, followed by detecting each of the detectable labels.

In one embodiment, the agent which selectively binds to a marker may be an antibody, and the detectable label may be a fluorophore. In this case, the expression of a marker is indicated by the wavelength and intensity of fluorescence emitted from the labelled pluripotent stem cell-derived cardiac cell. In some embodiments, the antibody may be an anti-CD200, anti-CD172a or anti-CD90 antibody, which is conjugated to a fluorophore. Many anti-CD200, anti-CD172a, and anti-CD90 antibodies, which are conjugated to a fluorophore, are known to the skilled person and available commercially, any of which is suitable to use in the methods of the invention. Examples of suitable antibodies conjugated to fluorophores include anti-CD172a PECy5, anti-CD90 BV650, and anti-CD200 BV421.

In another embodiment, the agent which selectively binds to a marker may be an antibody, and the detectable label may be a magnetic bead. In this case, the expression of a marker is indicated by the response of the pluripotent stem cell-derived cardiac cell to a magnetic field. In some embodiments, the antibody may be an anti-CD200, anti-CD172a or anti-CD90 antibody, which is conjugated to a magnetic bead. Many anti-CD200, anti-CD172a, and anti-CD90 antibodies, which are conjugated to magnetic beads, are known to the skilled person and available commercially, any of which is suitable to use in the methods of the invention.

In one embodiment, determining whether CD200, and optionally other markers, are expressed on a surface of the pluripotent stem cell-derived cardiac cell may be carried out by genetic modification to incorporate a reporter gene in the pluripotent stem cell-derived cardiac cell, wherein the reporter gene is transcribed in conjunction with transcription of the gene for the marker, and the reporter gene encodes a detectable product. Thus the detectable product is produced when the gene for the marker is transcribed. For example, the reporter gene may encode green fluorescent protein (GFP) or luciferase. If the expression of multiple markers is to be determined, multiple distinct reporter genes may be required. Genetic modification techniques for introducing a reporter gene into a cell line are known to the skilled person, and may be carried out using commercially available kits. Any such technique is suitable for use with the methods of the invention.

In one embodiment, the step of determining whether CD200, and optionally other markers, are expressed on a surface of the pluripotent stem cell-derived cardiac cell is carried out using flow cytometry. Typically in this case, the agent which selectively binds to a marker is an antibody, and the detectable label is a fluorophore. Preliminary processing steps may be undertaken prior to determining whether CD200, and optionally other markers, are expressed on a surface of the pluripotent stem cell-derived cardiac cell. This may include compensation, staining with a viability marker (for example Zombie NIR), and manual gating to remove artefacts including debris, dead cells and doublets.

Flow cytometry enables the detection of labelled pluripotent stem cell-derived cardiac cells amongst a large number of cells. Typically, appropriate fluorescence intensity thresholds (‘gates’) are set by the operator for a control sample, wherein if fluorescence levels of a cell exceed these gate levels, the cell is counted as a labelled pluripotent stem cell-derived cardiac cell. The fluorescence intensity values for the gates are dependent on the antibody label and will differ from one flow cytometer to another, as well as between individual runs and between different batches of antibodies. A commonly used guideline is to set the gates such that 0.5% or less of events for the control sample fall outside each gate. These gates are then applied to the sample to be measured, i.e. the sample containing pluripotent stem cell-derived cardiac cells that have been exposed to an antibody comprising a detectable label which is a fluorophore.

In an eighth aspect of the invention, there is provided a method of providing a substantially pure dose of pluripotent stem cell-derived ventricular cardiomyocytes (PSC-VMs).

In the method of the eighth aspect of the invention, a plurality of pluripotent stem cell-derived cardiac cells are subjected to the method of the seventh aspect of the invention as described above, to identify the PSC-VMs. That is, it is determined which of the pluripotent stem cell-derived cardiac cells express CD200 (and optionally other markers such as CD172a and/or CD90) on a surface thereof, wherein a pluripotent stem cell-derived cardiac cells which does not express CD200 on a surface thereof (and optionally expresses CD172a and/or does not express CD90) is identified as a PSC-VM.

Secondly, in the method of the eighth aspect of the invention, the PSC-VMs are isolated from the plurality of pluripotent stem cell-derived cardiac cells to provide a substantially pure dose of PSC-VMs. The PSC-VMs may be isolated from the plurality of pluripotent stem cell-derived cardiac cells using any suitable method known to the skilled person. In one embodiment, the PSC-VMs are isolated from the plurality of pluripotent stem cell-derived cardiac cells using fluorescence-activated cell sorting (FACS). In this case, in the method of identifying a PSC-VM, the pluripotent stem cell-derived cardiac cells are exposed to an agent containing a detectable label, wherein the agent selectively binds to the marker, which is an antibody conjugated to a fluorophore. Typically, in a FACS system, individual cells are placed into droplets and, depending on the wavelength and intensity of fluorescence emitted from the cell, an electrical charge is applied to the droplet so that it can be deflected by an electromagnetic field for separate collection as required. In one embodiment, the PSC-VM are isolated from the plurality of pluripotent stem cell-derived cardiac cells using magnetic-activated cell sorting (MACS). In this case, in the method of identifying a PSC-VM, the plurality of pluripotent stem cell-derived cardiac cells are exposed to an agent containing a detectable label, wherein the agent selectively binds to the marker, which is an antibody conjugated to a magnetic bead. Typically, in a MACS system, cells are passed through a magnetized column. Those cells which are labelled with a magnetic bead are magnetized to the walls of the column, and unlabeled cells pass through the column, and are thus separated. The steps of identifying PSC-VMs and removing PSC-VMs may be performed sequentially, for example as occurs in a FACS system, or simultaneously, for example as occurs in a MACS system.

The method of the eighth aspect of the invention provides a dose of PSC-VM which is substantially pure. Substantially pure means that no more than 0.001% of pluripotent stem cell-derived cardiac cells in the dose are not PSC-VM, or that no more than about 0.005%, or than about 0.01, 0.05, 0.1, 0.5, 1, 2, 2.5, 5, 10, 15, or 20% of pluripotent stem cell-derived cardiac cells in the dose are not PSC-VM.

### 6. Reducing Arrhythmia by Modifying Cell Culture Conditions

In a ninth aspect of the invention, there is provided a method of eliminating or reducing arrhythmia following transplantation of a dose containing a plurality of PSC-CMs in a subject, by modifying the conditions in which the PSC-CM are cultured.

In a tenth aspect of there is provided a method of providing a dose of PSC-CM which is substantially free from arrhythmogenic PSC-CMs, by modifying the conditions in which the PSC-CM are cultured.

In an eleventh aspect of the invention, there is provided a method of providing a dose of PSC-CM which contains an increased proportion of non-arrhythmogenic PSC-CMs, by modifying the conditions in which the PSC-CM are cultured.

The inventors have surprisingly found that adding retinoic acid to the culture medium during PSC differentiation results in the production of a plurality of PSC-CMs which is enriched in the arrhythmogenic CD172a+/CD90−/CD200+ subpopulation. Conversely, adding a retinoic acid inhibitor during PSC differentiation, or carrying out PSC differentiation in the absence of vitamin A, may advantageously suppress the generation of the arrhythmogenic CD172a+/CD90−/CD200+ subpopulation, and/or increase the generation of the non-arrhythmogenic CD172a+/CD90−/CD200− subpopulation.

The culture medium may be any culture medium suitable for the differentiation of PSC into PSC-CM, a variety of which are known in the art and commercially available. The culture medium may be a chemically defined medium, a serum-based medium, a serum-free medium, or a xeno-free medium. Examples of suitable media include mTeSR media (such as mTeSR Plus basal medium supplemented with mTeSR Plus 5× supplement), TeSR-2 media, Essential 8 media, RPMI (Roswell Park Memorial Institute) media (such as RPMI 1640 supplemented with B27 supplement).

In the methods of the ninth and/or tenth aspects of the invention, a plurality of PSC are cultured and differentiated to provide PSC-CM. The differentiation may take place in a culture medium under conditions effective to suppress the generation of arrhythmogenic PSC-CMs, wherein arrhythmogenic PSC-CMs are PSC-CMs which express CD200 on a surface thereof. The culture medium may be a standard culture medium as described in differentiation protocols known to the skilled person, to which agents are added to suppress the generation of arrhythmogenic PSC-CMs, or from which vitamin A is removed.

In an embodiment, the arrhythmogenic PSC-CMs express CD200 on a surface thereof. In one embodiment, the arrhythmogenic PSC-CMs express CD200, and express CD172a and/or do not express CD90, on the surface thereof. In one embodiment, the arrhythmogenic PSC-CMs express CD200, and express CD172a, on the surface thereof. In one embodiment, the arrhythmogenic PSC-CMs express CD200, and do not express CD90, on the surface thereof. In one embodiment, the arrhythmogenic PSC-CMs express CD200, and express CD172a, and do not express CD90, on the surface thereof.

In the method of the eleventh aspect of the invention, a plurality of PSC are cultured and differentiated to provide PSC-CM. The differentiation may take place in a culture medium under conditions effective to increase the generation of non-arrhythmogenic PSC-CMs, wherein non-arrhythmogenic PSC-CMs are PSC-CMs which do not express CD200 on a surface thereof. The culture medium may be a standard culture medium as described in differentiation protocols known to the skilled person, to which agents are added to increase the generation of non-arrhythmogenic PSC-CMs, or from which vitamin A is removed.

In an embodiment, the non-arrhythmogenic PSC-CMs do not express CD200 on a surface thereof. In one embodiment, the non-arrhythmogenic PSC-CMs do not express CD200, and express CD172a and/or do not express CD90, on the surface thereof. In one embodiment, the non-arrhythmogenic PSC-CMs do not express CD200, and express CD172a, on the surface thereof. In one embodiment, the non-arrhythmogenic PSC-CMs do not express CD200, and do not express CD90, on the surface thereof. In one embodiment, the non-arrhythmogenic PSC-CMs do not express CD200, and express CD172a, and do not express CD90, on the surface thereof.

In the ninth to eleventh aspects of the invention, the conditions effective to suppress the generation of arrhythmogenic PSC-CMs may comprise addition of a retinoic acid inhibitor to the culture medium. Retinoic acid inhibitors are compounds which inhibit the functions of retinoic acid. Retinoic acid inhibitors may be, for example, antagonists or inverse agonists of retinoic acid receptors, or alternatively, inhibitors of enzymes responsible for the production of retinoic acid, such as aldehyde dehydrogenase. The retinoic acid inhibitor may be selected from the group consisting of 4-diethylaminobenzaldehyde (DEAB), 4-[(1E)-2-[5,6-dihydro-5,5-dimethyl-8-(2-phenylethynyl)-2-naphthalenyl]ethenyl]benzoic acid (BMS-493), (E)-4-[2-(5,6-dihydro-5,5-dimethyl-8-phenyl-2-naphthalenyl)ethenyl]-benzoic acid (BMS-189453), and disulfiram. The retinoic acid inhibitor may be DEAB. The retinoic acid inhibitor may be BMS-493. The retinoic acid inhibitor may be BMS-189453. The retinoic acid inhibitor may be disulfiram. In one embodiment, the retinoic acid inhibitor is added to the culture medium at cardiac mesoderm induction. In one embodiment, the retinoic acid inhibitor is added to the culture medium during cardiomyocyte differentiation. Specifically, the retinoic acid inhibitor may be added at between day 3 and day 7 of cardiomyocyte differentiation. The retinoic acid inhibitor may be added between days 3 to 5 of cardiomyocyte differentiation. The retinoic acid inhibitor may be added between days 3 to 7 of cardiomyocyte differentiation. The retinoic acid inhibitor may be added between days 5 to 7 of cardiomyocyte differentiation. The exact timing may vary depending on the differentiation protocol. In one embodiment, in 2D culture retinoic acid inhibitor may be added at Day 3 with Wnt inhibitor (IWP-2) or at Day 5 and left in culture until Day 7 or 9. In one embodiment, in 3D culture the retinoic acid inhibitor may be added 3 days after Wnt inhibition.

In the ninth to eleventh aspects of the invention, the conditions effective to suppress the generation of arrhythmogenic PSC-CMs may comprise removal of vitamin A from the culture medium. Removal of vitamin A may be complete removal, such that the culture medium contains no vitamin A, or partial removal, such that the culture medium contains a lower concentration of vitamin A than standard conditions. Removal of vitamin A may occur during cardiomyocyte differentiation, and specifically, during days 5 to 7 following Wnt inhibition. In one embodiment, the conditions effective to suppress the generation of arrhythmogenic PSC-CMs may comprise, simultaneously or sequentially, addition of a retinoic acid inhibitor to the culture medium and removal of vitamin A from the culture medium. For example, a retinoic acid inhibitor may be added to a standard culture medium at day 3 after mesoderm induction, followed by switching to a chemically-defined medium free of vitamin A at day 5. In one embodiment, retinoic acid receptors may have ligands other than vitamin A, thus removal of vitamin A may also be accompanied by addition of a retinoic acid inhibitor which is an antagonist or inverse agonist of retinoic acid receptors, such as BMS-493 or BMS-189453.

In the method of the ninth aspect of the invention, arrhythmia may be eliminated or reduced following transplantation of the dose of PSC-CM, compared to arrhythmia following transplantation of a dose of PSC-CM which has not been subjected to the method of the ninth aspect of the invention. This may be assessed in practice by comparing arrhythmia experienced by a test subject who received the purified dose, to arrhythmia experienced by a control subject who received the crude dose. Arrhythmia may be reduced as defined by the cumulative time per day spent in arrhythmia over 25 days post-transplantation. Arrhythmia may be reduced by at least about 10%, or at least about 20, 30, 40, 50, 60, 70, 75, 80, 85, 90, 95, 99 or at least about 100%. Arrhythmia may be eliminated.

The method of the tenth aspect of the invention provides a dose of PSC-CM which is substantially free from arrhythmogenic PSC-CM. The dose of PSC-CM which is substantially free from arrhythmogenic PSC-CMs may contain less than 0.001% of arrhythmogenic PSC-CMs, relative to the total number of PSC-CM in the dose. The dose of PSC-CM which is substantially free from arrhythmogenic PSC-CMs may contain less than 0.005%, or less than about 0.01, 0.05, 0.1, 0.5, 1, 2, 2.5, 5, 10, 15, or 20% of arrhythmogenic PSC-CMs, relative to the total number of PSC-CM in the dose.

In the method of the eleventh aspect of the invention a dose of PSC-CM is provided which contains an increased proportion of non-arrhythmogenic PSC-CMs. Non-arrhythmogenic PSC-CMs are as described above. An increased proportion of non-arrhythmogenic PSC-CMs means that the proportion of non-arrhythmogenic PSC-CMs contained in the dose is at least about 5% greater than the proportion of non-arrhythmogenic PSC-CMs contained in a control dose. The proportion of non-arrhythmogenic PSC-CMs contained in the dose may be at least about 10, 20, 30, 40, 50, 60, 70, 80, 90, or about 95% greater than the proportion of non-arrhythmogenic PSC-CMs contained in a control dose. The proportion is calculated relative to the total number of PSC-CM in the dose or the control dose, as appropriate. The increase refers to an increase in the proportion. For example, if a control dose contains 10% non-arrhythmogenic PSC-CMs, a dose of PSC-CM provided according the eleventh aspect of the invention having 5% more non-arrhythmogenic PSC-CMs contains at least 15% non-arrhythmogenic PSC-CMs, i.e. an increase of 5%. The control dose is a dose prepared in the absence of conditions effective to increase the generation of non-arrhythmogenic PSC-CMs, i.e. using standard culture media. For example, if the conditions effective to increase the generation of non-arrhythmogenic PSC-CMs comprise addition of a retinoic acid inhibitor to the culture medium, then the control dose is prepared without the addition of a retinoic acid inhibitor.

### EXAMPLES

### Cellular Heterogeneity of PSC-CM Grafts is Linked to Treatable Arrhythmias

Current cardiac differentiation protocols yield heterogeneous cellular outputs. Although ventricular cardiomyocytes predominate, other cell types such as atrial and pacemaker-like cells along with non-myocytes are also present. Despite a widely postulated view that purified ventricular cardiomyocytes may be the most desirable cell product for transplantation applications, to date no study has robustly assessed how the composition of PSC-CM cell doses may impact EA burden. In this study, the inventors sought to understand the important relationship between cellular heterogeneity and arrhythmogenesis. The inventors also sought to test effective clinically available pharmacologic and procedural anti-arrhythmic treatments that could abrogate EAs should they arise in clinical trials. The inventors use a porcine MI model to study engrafted PSC-CMs after robustly phenotyping the composition of the input cells with particular focus on cardiomyocyte subpopulations. The inventors aimed to identify cellular characteristics predictive of arrhythmogenesis and hypothesised that this would yield data informative for the safe production of cardiomyocytes for future clinical use.

### Methods

### Cell Production

### Bioreactor Differentiation Protocol

The H9 cell line containing the gCaMP6f fluorescent calcium reporter was used for all experiments (provided by University of Queensland StemCore facility). Each production run was started from a frozen working cell bank (WCB) cryovial, which was expanded for 8 days in monolayer on matrigel (Corning) using the commercially available medium mTeSR™ Plus (Stem Cell Technologies) (FIG. 1). On Day −3, cells were inoculated at a density of 2.5-3.0×10⁵ cells/mL in mTeSR 3D supplemented with 10 μM Y-27632 (TOCRIS Bioscience) and 10 ug/mL of a thermoresponsive polymer pNIPAM conjugated to fibronectin (Chen, X. et al. Tissue Eng Part C Methods 24, 146-157) to form aggregates. A DASbox (Eppendorf) stirred tank bioreactor system was used for aggregate formation and suspensions controlled at 37.2° C., pH 7.2, and 30% DO. At day −1, rapamycin (Merck) was added to a final concentration of 5 nM to enhance survival during differentiation. On day 0, pluripotent aggregates were washed twice with RPMI 1640 then cultured in RPMI-B27 without insulin (Thermo Fisher Scientific) containing 6 μM CHIR 99021 (TOCRIS Bioscience) and 5 nM of Rapamycin. On day 1, 24 hours post mesoderm induction, aggregates were washed once with RPMI 1640 and returned to RPMI-B27 without insulin, containing 5 nM Rapamycin. On day 2, aggregates were washed and returned to RPMI-B27 without insulin, containing 2 μM IWP-2 (Stem Cell Technologies). On day 4, aggregates were transferred to RPMI-B27 with insulin and media exchanged every other day until cryopreservation at day 15. Prior to cryopreservation, H9-gCaMP6f derived cardiomyocytes were heat-shocked for 30 minutes at 42° C. and treated with a pro-survival cocktail (100 ng/mL IGF-1, 0.6 μM Cyclosporin A) to enhance their survival after transplantation. Cardiomyocyte aggregates were incubated for 6 hours in 4 mg/mL Collagenase IV, washed, and dissociated to single cells using TrypLE (Thermo Fisher). Cardiomyocytes were cryopreserved at 10×10⁶ cells/mL in CryoStor CS10 (Stem Cell Technologies).

### Flow Cytometry

The expression of cTnT was measured on a Cytoflex Flow Cytometer (Beckman Coulter). Briefly, 1×10⁶ cells were fixed with 2% paraformaldehyde for 10 min at room temperature. Cells were stored in FACS wash buffer (0.5% BSA in PBS) at 4° C. until staining. Samples were permeabilised in 0.1% Triton X-100 for 10 min then stained with CTNT-FITC 1:50 (Miltenyi Biotec) for 30 min at room temperature. Cells were washed twice before analysis. The excitation laser and emission filters used were: Ex. 488, Em. 525/40.

### Quantitative PCR

After dissociation of aggregates, 1×10⁶ cells were collected in RNAprotect Cell Reagent (Qiagen) and stored at 4° C. until RNA extraction. RNA was extracted using the Qiagen RNeasy Mini kit according to manufacturer's instructions. Using RevertAid First Strand cDNA Synthesis Kit (ThermoFisher Scientific), 1 μg of RNA was converted to cDNA in a 20 μL reaction according to manufacturer's instructions. cDNA was diluted 1:10 with DNAse/RNAse free water and 1 μL of diluted cDNA used in each 10 μL reaction with 4 μL of 1 M F/R primers and 5 μL Fast SYBR Green qPCR master mix (ThermoFisher Scientific). Genes and respective primers used are outlined in Table 1.

TABLE 1
Genes and primers used for qPCR experiments.

| Gene | Forward Primer | Reverse Primer |
|---|---|---|
| OCT3/4 | AGCGATCAAGCAGCGACTAT | AGAGTGGTGACGGAGACAGG |
| NANOG | ACCTTCCAATGTGGAGCAAC | GAGAATTTGGCTGGAACTGC |
| DNMT3B | TCCTCAAAGAGTTGGGCATAA | TTTGATATTCCCCTCGTGCT |
| HAND1 | GCCTAGCCACCAGCTACATC | ATCCGCCTTCTTGAGTTCAG |
| GATA6 | AGAGCACCAATCCCGAGAAC | GCACGGAGGACGTGACTT |
| EOMES | ACCCCCTTCCATCAAATCTC | CCATGCCTTTTGAGGTGTCT |
| MESP1 | AGCCCAAGTGACAAGGGACAACT | AAGGAACCACTTCGAAGGTGCTGA |
| Brachyury (T) | CCTTGCTCACACCTGCAGTAGC | GGCCAACTGCATCATCTCCA |
| ISL1 | GAAGGTGGAGCTGCATTGGTTTGA | TAAACCAGCTACAGGACAGGCCAA |
| SIRPA | ACCTGGCTCAGGCTAGTTCCAAAT | TGTGCACACGTATGTGCTGTCTCT |
| CTNT | TTCACCAAAGATCTGCTCCTCGCT | TTATTACTGGTGTGGAGTGGGTGTGG |
| MYH6 | TCAGCTGGAGGCCAAAGTAAAGGA | TTCTTGAGCTCTGAGCACTCGTCT |
| MYL7 | ACATCATCACCCATGGAGACGAGA | GCAACAGAGTTTATTGAGGTGCCC |
| MYH7 | TCGTGCCTGATGACAAACAGGAGT | ATACTCGGTCTCGGCAGTGACTTT |
| NXK2-5 | TTTGCATTCACTCCTGCGGAGACCTA | ACTCATTGCACGCTGCATAATCGC |
| MYL2v | TGTCCCTACCTTGTCTGTTAGCCA | ATTGGAACATGGCCTCTGGATGGA |
| NPPA | GGGTCTCTGCTGCATTTGTGTCAT | AGAGGCGAGGAAGTCACCATCAAA |
| KCNJ3 | TCATCAAGATGTCCCAGCCCAAGA | CACCCGGAACATAAGCGTGAGTTT |
| HCN4 | TCTTCCTCATTGTGGAGACACGCA | TGAGGATCTTCGTGAAGCGGACAA |
| SHOX2 | AAGAGGATGCGAAAGGGATG | TGAGTTGTTCCAGGGTGAAAT |

Reactions were performed in technical triplicate. Quantitative PCR was performed on the BioRad CFX96 Real-Time PCR Detection System with standard cycling parameters described in the master mix protocol. Dissociation curves were acquired at the conclusion of each run. Fold difference in expression was calculated using the comparative CT formula, using GAPDH cDNA from a reference sample. Depending on the gene and time point, undifferentiated hPSCs or fetal heart RNA were used as the reference sample.

### Single-Cell RNA Sequencing

### Hashtagging and Sequencing Protocol

Frozen PSC-CMs were thawed in the water bath and the content of each vial was transferred to a 50 mL Falcon tube containing RPMI-B27 medium with insulin (ThermoFisher Scientific), 5% FBS (ThermoFisher Scientific), and 10 uM ROCK inhibitor Y-27632 (STEMCELL Technologies). Each sample was spun down at 1000 rpm for 5 mins and the cell pellet was resuspended with filtered PBS plus 2% BSA. Cells were then incubated at room temperature for 10 mins in blocking buffer containing 5 μL of Human TruStan FcX solution (BioLegend) plus 100 μL of staining buffer (2% BSA+0.01% Tween20 in filtered PBS). Next, 1 μL of hashtag antibody (BioLegend) was added to each sample and incubated on ice for 20 mins. The hashtagging protocol employed allowed for multiple samples to be pooled together in a single sequencing run. Cells were then washed twice with staining buffer and 4×10⁵ cells from each stained sample were collected and pooled into a 2 mL Eppendorf tube. Prior to sequencing, a viability test suggested an 80% viability of the captured cells. The cell suspensions were loaded onto 10× Genomics Single Cell 3′ Chips to form single cell gel beads in emulsion (GEMs). The Chromium library pool containing 90% gene expression library with 10% hashing library spike-in generated and sequenced on Illumina NextSeq 500 instrument. The sequencing data were further processed to generate FASTQ files and the raw count matrix using the CellRanger pipeline at the Sequencing Facility at the University of Queensland. The multiplexed samples were demultiplexed to their original identities by mapping of sample reads to GRCh38-3.0.0 human reference genome and hashing sequences.

### Bioinformatics Analysis

The gene expression count matrix was loaded in Seurat. The inventors removed genes that were expressed in less than 1% of total cells prior to creating the Seurat object. The hashtag matrix was added to Seurat object as a new assay separate from RNA. RNA data was normalised with log normalisation and the cell hashing data was normalised with centered log-ratio (CLR) transformation. Following normalisation, cells were demultiplexed and mapped to their original sample identities or assigned as doublets or negatives based on the Seurat HTODemux algorithm. In addition, the inventors converted Seurat object to SingleCellExperiment and run single cell doublet scoring (scds) for doublet annotation. Scds method returns 3 scores whereas HTODemux returns just one mark. Cells were considered as doublet and filtered out when annotated as doublet by at least two of the classification. The inventors further filtered the cells following the standard quality control (QC) workflow in Seurat. The inventors removed low-quality cells after pre-processing the data and retained 11307 cells for continued analysis. After normalisation and scaling of the data, the inventors performed dimensional reduction and selected 50 principal components (PC) dimensions as input to the unsupervised clustering and uniform manifold approximation and projection (UMAP) plot. Cell type demarcating each cluster was annotated by looking into the expression level of marker genes. Nebulosa package was used for enhanced visualisation of marker genes expression.

### High Parameter Flow Cytometry

### Antibody Staining

Thawed PSC-CMs (˜1.5×10⁶ cells per sample tube) were stained with an amine reactive viability dye (Zombie NIR, Biolegend) for 30 minutes in PBS. The samples were washed twice in PBS+2% FBS and centrifuged at 225 G each time. The samples were then stained with fluorochrome-conjugated membrane marker antibodies as set out in Table 2 below for 30 minutes in PBS+2% FBS and washed twice.

TABLE 2
Antibody panel.

| Company | Marker | Clone | Fluorophore | Dilution | Significance |
|---|---|---|---|---|---|
| | GCaMP | | eGFP | | Genetically encoded, intracellular |
| | | | | | calcium transient marker |
| BD Biosciences | VCAM-1 | 51-10C9 | PerCP-Cy5.5 | 1:100 | hPSC-CM lineage marker |
| | | | | | CD172A⁺/VCAM1⁺ phenotype |
| | | | | | indicates committed CM |
| Biolegend | SIRPα | SE5A5 | PECy5 | 1:100 | hPSC-CM marker |
| ThermoFisher | Desmoglein | CSTEM28 | PE | 1:200 | Desmosomal protein, highly |
| Scientific | 2 | | | | expressed in CMs |
| Biolegend | CD31 | WM59 | PECy7 | 1:100 | Endothelial cell marker |
| BD Biosciences | CD235a | GA- | BUV395 | 1:100 | Early marker of mesoderm that |
| | | R2(HIR2) | | | gives rise to ventricular CM's |
| Biolegend | CD90 | 5E10 | BV650 | 1:100 | Cardiac fibroblast marker, |
| | | | | | although non-specific (also |
| | | | | | expressed on endothelium, |
| | | | | | mesenchymal stem cells) |
| Biolegend | CD77 | 5B5 | BV510 | 1:100 | CD77⁺/CD200⁻ suggests |
| | | | | | ventricular CM phenotype |
| Biolegend | CD13 | WM15 | BV786 | 1:100 | Early cardiac mesoderm marker |
| Biolegend | CD200 | OX-104 | BV421 | 1:100 | CD77⁺/CD200⁻ suggests |
| | | | | | ventricular CM phenotype |
| Biolegend | CD34 | 581 | AF700 | 1:100 | Endothelial cell and progenitor |
| | | | | | marker |
| Abcam | Vinculin | EPR8185 | AF647 | 1:200 | Adhesion marker, links CM |
| | | | | | contractile apparatus to adherens |
| | | | | | junctions (intracellular) |
| BD Biosciences | Cardiac | 13-11 | BV421 | 1:20 | A regulatory protein of the |
| | Troponin T | | | | cardiomyocyte contractile |
| | | | | | apparatus and pan-CM marker |
| | | | | | (intracellular) |
| Biolegend | Zombie | | NIR | 1:1000 | Live/dead stain |
| | Viability | | | | |

For measurement of post-thaw cardiac Troponin T positive cells, separate samples of PSC-CMs were fixed for 20 minutes with 4% PFA and permeabilised in PBS+0.5% Tween-20. The cells were incubated with anti-human Troponin T antibody in permeabilization buffer for 30 minutes then washed twice in PBS+2% FBS.

Single colour compensation controls were created using compensation beads as follows: CompBead Plus, BD, for mouse antibodies; AbC Total Antibody, ThermoFisher, for rabbit antibodies; ArC amine reactive beads, ThermoFisher, for Zombie NIR and unstained PSC-CMs for endogenous GCaMP/GFP.

### Data Acquisition and Analysis

PSC-CMs and compensation controls were analysed with a BD FACSymphony A5 cytometer and FACS Diva software, using application settings.

Data analysis was performed using FlowJo software (Treestar, Version 10.7.2). An overview of the manual gating and data processing method is shown in FIG. 2. Compensated data was manually gated to remove debris, non-viable cells, and doublets. To identify PSC-CM sub-populations based on surface marker expression, dimensionality reduction was performed using t-distributed stochastic neighbour embedding (tSNE), and unsupervised clustering was performed using FlowSOM (opt-SNE parameters: Gradient algorithm—Barnes-Hut, Learning configuration—opt-SNE, KNN algorithm—exact vantage point tree, Iterations—1000, Perplexity—30; FlowSOM parameters: Number of meta-clusters—25, SOM grid size—10×10, Node scale—100%, Set Seed—3). To compare dose composition between animals and identify potentially pro-arrhythmogenic sub-populations, the inventors downsampled and concatenated the data prior to dimensionality reduction and clustering.

### In Vitro Electrophysiology

iPSC-CM Monolayer Differentiation

iPSC-CMs (SCVI-8, Stanford Cardiovascular Institute) were used for in-vitro electrophysiology experiments and were differentiated in 2D monolayer culture. Two days prior to differentiation, PSCs were seeded into Matrigel-coated six-well culture plates, at a density of 1.5×10⁶ cells per well, in mTeSR Plus (STEMCELL Technologies)+10 μM Y-27632. On the first day of differentiation (day 0), media was changed from mTeSR Plus to RPMI 1640+B27 without insulin+Glutamax+Penicillin/Streptomycin+6 μM of the GS3K inhibitor, CHIR-99021 (Tocris Bioscience). After 24 h, the cells were washed with PBS to remove CHIR-99021 and the media was switched to RPMI 1640+B27 without insulin+Glutamax+Penicillin/Streptomycin. On day 3, the media was replenished and supplemented with Wnt signalling inhibitor, IWP-2 (5 μM). On day 5, media was changed to RPMI 1640+B27 without insulin+Glutamax+Penicillin/Streptomycin. For atrial/pacemaker cell enrichment differentiations, 1 μM retinoic acid (Sigma-Aldrich) was added at media change on day 3 and again on day 5. On day 7, media was changed to RPMI 1640+B27 with insulin+Glutamax+Penicillin/Streptomycin. Following this, media was changed every 2-3 days. Cardiomyocyte beating commenced between days 6 and 7.

### Patch Clamping

For electrophysiological measurements using high-throughput patch clamping, beating iPSC-CMs were dissociated with TrypLE (ThermoFisher) on day 15 and replated onto Matrigel-coated 12 well plates at a density of 1.5×10⁶ cells per well and maintained in culture until being used for patch-clamping experiments between days 30-35. On the day of patch clamp recording, the cells were washed with PBS, dissociated with TrypLE, and centrifuged for 3 minutes at 300 G. The cells were then resuspended in a solution of 80% RPMI-1640 without phenol red+Ca²⁺+10% FBS and 20% divalent cation free buffer containing 140 NaCl, 4KCl, 5 Glucose, 10 HEPES, pH7.4 with NaOH. The cells were counted and diluted to a concentration of ˜2×10⁵ cells/mL using the divalent free solution.

Patch clamp recordings were collected using a Syncropatch 384PE (Nanion Technologies), in voltage clamp mode. Sodium current (I_{Na}) was recorded with internal and external solutions containing the following (mM): 110 CsF, 10 NaCl, 10 CsCl, 10 HEPES, 10 EGTA ph7.2 with CsOH and 140 NaCl, 4 KCl, 5 glucose 10 HEPES 2 CaCl₂), 1 MgCl₂ pH7.4 with NaOH, respectively. Sodium current was elicited in 5 mV voltage steps ranging from −120 to +40 mV from a holding potential of −80 mV, with a 200 ms pre-pulse to −120 mV.

### Optical Electrophysiology

Action potentials were recorded using a kinetic imaging cytometer (KIC, Vala Sciences, San Diego, CA. USA) and voltage-sensitive fluorescent indicator (FluoVolt, ThermoFisher). On day 14 of differentiation, beating cardiomyocytes were dissociated with TrypLE (ThermoFisher) and replated onto Matrigel-coated flat-bottomed 96 well plates (Greiner CELLSTAR, Sigma-Aldrich) at a density of 1.5×10⁴ cells per well. The cells were maintained for a further 5-7 days. On the day of recording, the cells were washed with 100 μL RPMI-1640 without phenol red+Ca²⁺. The media was removed and then replaced with 50 μL fresh RPMI without phenol red+Ca²⁺ and incubated at 37° C. for 60 minutes. The cells were then incubated for 20 minutes in RPMT-1640 without phenol red supplemented with FluoVolt, Powerload pluronic solution, and Hoescht dye. The FluoVolt solution was then removed and replaced with fresh RPMI-1640 without phenol red+Ca²⁺.

The cells were recorded on the KIC for 5 seconds unstimulated, followed by 10 seconds stimulation at 1.5 Hz, followed by another 5 seconds unstimulated. Cell segmentation and single cell transient extraction was performed with CyteSeer Scanner software (Vala Sciences, San Diego, CA. USA). The optical action potential measurements were analysed using custom KIC data analysis software (KIC DAT)⁷⁰.

### Porcine Experiments

### Acclimatation/Housing

All experiments were conducted in female landrace swine (2 to 4 months, 25-30 kg) acquired from the same local source. All procedures in this study were approved by the Western Sydney Local Health District Animal Ethics Committee (protocol ID: 4262.03.17). Animals were housed in a purpose-built large animal research facility to which they were brought 1-2 weeks prior to the first scheduled procedure for acclimatization.

### Myocardial Infarction

Myocardial infarction was percutaneously induced by methods previously described (Thavapalachandran, S. et al. Science Translational Medicine 12, eaay2140 (2020)). Briefly, animals were premedicated with intramuscular ketamine (10 mg/kg), methadone (0.3 mg/kg), and midazolam (0.3 mg/kg), intubated, ventilated, and maintained with inhalational isoflurane. The left coronary artery was engaged percutaneously via the right femoral artery using a 6F hockey stick guiding catheter (Medtronic, Minnesota, U.S.A). A 0.36-mm coronary guidewire (Sion Blue, Asahi Intecc Co. Ltd., Aichi, Japan) was delivered into the left anterior descending artery (LAD). Myocardial infarction was induced by occluding the mid LAD distal to the first diagonal branch with an inflated 2.0-3.0-mm angioplasty balloon (Boston Scientific, Massachusetts, U.S.A) for 90 minutes. Coronary angiography performed after reperfusion confirmed vessel patency and resolution of ST elevation. Ventricular arrythmias were treated with anti-arrhythmics and defibrillation as required.

### Telemetry Device Implantation

All animals underwent telemetry transmitter implantation (easyTEL+, Emka technologies) under general anaesthesia. The transmitter was implanted in a subcutaneous pocket which was created in the left flank. Subcutaneous leads were tunnelled to capture the cardiac apex and base. Signal quality was assessed prior to securing device and leads in situ.

### Telemetry Analysis

Telemetric ECG and accelerometer data was continuously monitored from the time of device implantation. Semi-automated quantification of heart rate, arrhythmia burden and accelerometer data was performed offline by a cardiologist using the EcgAUTO 3.5.5.16 software package (Emka technologies, Paris, France). Arrhythmia was defined as an ectopic beat (e.g. premature ventricular contraction) or rhythm. The entire recorded dataset for each subject was analysed, with data presented as daily averages (mean±S.E.M).

### Cardiac MRI

Animals were premedicated with intramuscular ketamine (10 mg/kg), methadone (0.3 mg/kg), and midazolam (0.3 mg/kg), before intubation and mechanical ventilation. General anesthesia was induced with intravenous propofol (2-5 mg/kg) and maintained with 2% inhaled isoflurane. Breathing was held in end-expiration for all image acquisitions. All CMR examinations were performed on a Siemens 3T Prisma system (Siemens Medical Systems) utilising an 18-channel body array together with spine array coils and 4-lead electrocardiogram (ECG) gating. Axial and coronal TrueFISP (true fast imaging with steady state precession) sequences through the heart were acquired to plan preliminary 2-chamber, short-axis, and 4-chamber single-slice gradient images. True 2-chamber, 3-chamber, 4-chamber, and right ventricular outflow tract 8 mm single-slice TRUFI (true fast imaging) cines were then planned and acquired, as well as a short-axis stack of 14 contiguous 8 mm slices, starting just beyond the apex and extending into the atrium, planned from 2- and 4-chamber cine images in end-diastole.

TrueFISP imaging was used for all acquisitions with the following parameters: TE (echo time) 1.3 ms, TR (repetition time) at R-R interval of individual animal, FOV (field-of-view) 370 mm, slice thickness 8 mm, in-plane resolution 1.4 mm×1.4 mm, flip angle 10 degrees, 25 calculated phases.

Image analysis was performed offline using dedicated software (Medis Suite MR 3.2, Medis Medical Imaging, Schuttersveld 9, The Netherlands) by two independent, blinded cardiologists. Volumetric assessment was performed according to the Society for Cardiovascular Magnetic Resonance guidelines. In brief, short axis end-diastolic and end-systolic images were chosen as the maximal and minimal mid-ventricular cross-sectional areas. End-diastolic epicardial and endocardial borders, along with end-systolic endocardial borders were manually traced for each slice. The vendor specific automated contouring algorithm was not used due to suboptimal performance in non-human subjects. Papillary muscles were included in volume and excluded in mass calculations. The difference between end-diastolic and end-systolic endocardial borders represented the left or right ventricular stroke volume, and ejection fraction was calculated as the stroke volume/end-diastolic volume. All parameters were analysed by two independent, blinded observers with excellent inter-observer variability. Intra-observer variability was determined by having one observer, blinded to previous measurements, repeat measurements on all subjects on two separate occasions, at least two weeks apart.

### ADAS-3D CMR Reconstructions

Following CMR image acquisition, A separate offline segmentation software, ADAS-3D (Galgos Medical, Barcelona, Spain), was utilized to process 3D reconstructions of the left ventricle (LV) and fibrosis as identified on LGE for integration in to the electroanatomic mapping system (EAM). Complete cardiac anatomy including the coronary arteries were also exported to assist with registration to EAM reconstructions. Endocardial and epicardial borders were delineated in all slices and an assessment of scar was made based on the pixel signal intensity (PSI) between the two borders. Values for the identification of dense fibrosis and border zone regions using the maximum PSI have been previously correlated with low voltage regions and conducting channels on EAM. Fibrosis area was calculated by averaging the PSI of the endocardial to mid myocardial layers and the average PSI of the mid myocardial to epicardial layers. The calculated values were then projected using the trilinear interpolation method to the LV reconstruction of the endocardial and epicardial surfaces.

### Thoracotomy, Epicardial Mapping and Cell Injection

On day 0, animals were sedated and returned to the operating theatre for thoracotomy and transepicardial injections. Following cannulation and intubation, an arterial line was inserted into the auricular or distal limb artery to enable continuous blood pressure monitoring. A 100 mcg Fentanyl patch was applied, and intravenous antibiotic (Cephazolin, 1 g) was administered. Intercostal nerve block of the 2^{nd} to 6^{th} rib spaces was achieved with Bupivacaine and Lignocaine.

Thoracotomy was performed by making an incision in the left lateral chest wall at the 4^{th}/5^{th} intercostal space. The incision was opened under direct vision using self-retaining rib retractors. The pericardium was opened anteriorly, and the apex and anterior left ventricle were gently exposed by packing the posterior mediastinum with wet gauze. Haemodynamics were carefully monitored and metaraminol boluses were administered as required to maintain the systolic blood pressure greater than 100 mmHg. The epicardial surface of the left ventricle was electroanatomically mapped using an electrophysiological mapping catheter (Navistar Thermocool Smarttouch, Biosense Webster). Cardiac MRI was imported to the EAM system and registration was performed by placing the mapping catheter placed at fiducial landmarks under direct visualization. Fiducial landmarks used included the apex, mitral valve annulus and left anterior descending artery. EAM points were acquired on the EAM system with mapping catheter and identical landmarks were selected on the cardiac MRI. An initial registration was performed with landmarks and then a secondary registration with a best fit of all anatomy was applied using the EAM system software (CartoMerge, Biosense Webster). Confirmation of registration accuracy was reconfirmed with the EAM catheter. Following registration, the electroanatomic voltage map was assessed to identify scar, border and remote zones. Transepicardial injections of either vehicle (8×300 μL injections of RPMI B27) or cells (750×10⁶ cells distributed in 8×300 μL injections) were then performed under direct vision into infarct and border zones using a 27-gauge insulin syringe.

### Central Venous Catheter Insertion and Immunosuppression

All subjects received a three-drug immunosuppressive regimen to prevent xenograft rejection. Commencing 5 days prior to cell injections, animals received oral cyclosporine A (10-15 mg/kg twice daily) aiming to maintain trough levels greater than 250 ng/mL. On day 0, a 2-lumen 5 French central venous catheter (Teleflex) was implanted in the right jugular vein under ultrasound guidance to facilitate ongoing administration of intravenous immunosuppressants and regular blood draws. Following central line placement and prior to cell injection, 500 mg Abatacept (CTLA4-Ig, Bristol-Myers Squibb), 30 mg/kg methylprednisone and 3-5 mg/kg cyclosporine A was administered intravenously. From day 1 post transplantation until euthanasia, subjects received twice daily oral cyclosporine A (10-15 mg/kg) to maintain trough levels greater than 250 ng/mL, along with 100 mg intravenous methylprednsione daily. Another 250 mg dose of intravenous Abatacept was given 2 weeks post transplantation. Prophylactic oral amoxicillin/clavulanic acid was given daily to all subjects to prevent central line and thoracotomy infections, and 30 mg oral lansoprazole daily was given for gastrointestinal protection.

### Anti-Arrhythmic Treatment

The animals randomised to anti-arrhythmic treatment received a 150 mg IV amiodarone bolus at the time of cell injection. From day 1 post transplantation until euthanasia, they were treated with a regimen of 200 mg oral amiodarone twice daily and 10 mg oral ivabradine twice daily.

### Electrophysiological Study

Prior to euthanasia, all subjects underwent electrophysiological study under general anaesthesia to determine inducibility, mechanism, and electroanatomic origin of any ventricular arrhythmia. An 8.5 Fr Agilis steerable introducer (Abbott Medical) was inserted into the right common femoral vein under ultrasound guidance through which an Advisor HD Grid (Abbott Medical) multi-electrode electrophysiological catheter was advanced to the right ventricle. A 6 Fr decapolar electrophysiological catheter was positioned into the coronary sinus via the right femoral vein. The Ensite Precision EAM system (Abbott Medical) was used to generate a substrate map delineating scar, border and remote zones using bipolar and unipolar voltage cutoffs of 0.5-1.5 mV and 3-8.3 mV respectively. The left ventricle was then mapped via a retrograde aortic approach. A substrate map was initially created if the subject was in sinus rhythm. Following substrate mapping, VT induction via programmed electrical stimulation (PES) was performed. A drive train of eight beats at 400 ms was followed by up to four extra-stimuli delivered one at a time. Initial extrastimuli were delivered at a coupling interval of 300 ms which was then decreased by 10 ms until ventricular refractoriness. A modified “Michigan” protocol was additionally performed. The Michigan protocol uses exclusively four extra stimuli. At each drive train cycle length of 350 ms, programmed stimulation is initiated with coupling intervals of 290, 280, 270, and 260 ms for the first through fourth extrastimuli. The coupling intervals of the extra stimuli are shortened simultaneously in 10-ms steps until an extrastimuli is refractory or an arrhythmia is induced. Both PES protocols were repeated at two sites. Following PES an isoprenaline bolus (20 mcg) and infusion (6-10 mcg/min) was administered with burst pacing starting from 300 ms and reduced in 20 ms steps until refractory during isoprenaline delivery and washout. The mechanism of any spontaneous or induced ventricular arrhythmias was elucidated through standard pacing maneuvers followed by EAM to identify arrhythmia origin.

### Catheter Ablation

Subjects who underwent catheter ablation for treatment of EA were premedicated with intramuscular ketamine (10 mg/kg), methadone (0.3 mg/kg), and midazolam (0.3 mg/kg), intubated, ventilated, and maintained with inhalational isoflurane. An arterial line was inserted into the auricular or distal limb artery to enable continuous blood pressure monitoring. All subjects were in spontaneous engraftment arrhythmia at the time of the ablation procedures. Arrhythmia origin was electroanatomically mapped as described above. At site of earliest activation mapping, radiofrequency ablation lesions were delivered using a 4 mm tip open-irrigation catheter (Flexability, Abbott Medical). A single grounding patch was placed on each animal and ablation was performed in power control mode using 30-40 W of power and 13 mL/min irrigation flow rate with normal saline. Delivery of each lesion was attempted for 30-60 seconds unless terminated prematurely due to catheter movement or impedance rise. Ablations were terminated after sinus rhythm was restored, followed by an attempt at VT induction as described above.

### Euthanasia and Tissue Harvest

Following the terminal EPS procedure, subjects were euthanised with potassium chloride (75-150 mg/kg) and hearts were excised and fixed in 10% neutral buffered formalin for subsequent analysis.

### Histology

### Tissue Processing

Pig hearts were fixed whole in 10% neutral buffered formalin for a minimum of 48 hours. The ventricles were then sliced into transverse sections of approximately 1 cm thickness from the apex (Level 1) towards the base (Level 7). After fixation, the tissues were switched to 70% ethanol, processed and paraffin embedded. Processing, embedding, and sectioning of Level 1 blocks was performed at the Westmead Institute for Medical Research. Processing, embedding, and sectioning of large blocks (Levels 2 and above) was performed by Veterinary Pathology Diagnostics Services at the University of Sydney.

### Immunohistochemistry

Immunohistochemistry analyses were performed on 4 μm sections taken from Levels 1, 2, or 3. Sections were deparaffinised in xylene and hydrated by sequential incubation in 100%, 95%, 70%, 50% ethanol, and water. Antigen retrieval was performed using heated sodium citrate buffer and the sections were then washed in PBS+0.1% Tween 20, blocked with 5% goat serum in PBS+0.5% Tween-20, and stained with primary antibodies overnight at 4° C. The following day, the sections were washed and incubated with secondary antibodies for one hour at room temperature, in the dark. The sections were then washed again and incubated with DAPI (1 μg/mL, Sigma-Aldrich/Merck) for 10 minutes, rinsed with PBS and mounted with PBS: Glycerol. For demonstration of PSC-CM grafts, and radiofrequency ablation site, whole mount sections from Level 2 tissue blocks were stained with primary antibodies targeting human Ku80, and cardiac troponin T, as described above. For brightfield detection of secondary staining, the inventors used the ImmPRESS Duet double staining polymer kit HRP/AP (Vector Laboratories, MP-7714), and then counterstained sections with aniline blue (1% aniline blue in MilliQ water for 1 minute).

### Imaging and Analysis

Immunofluorescence and brightfield microscopy were performed on an Olympus VS120 Slide Scanner, with the 20× objective (UPLSAPO 20×/NA 0.75, WD 0.6/CG Thickness 0.17 or the 40× objective (UPLSAPO 40×/NA 0.95, WD 0.18/CG Thickness 0.11-0.23). Images were acquired using Olympus VS-ASW 2.92 software and processed using Olympus VS-DESKTOP 2.9.

Confocal images of GFP, Cx43 and cTnT immunostained pig tissues were taken on a Leica SP8 microscope using tuneable white light and 405 nm lasers. Filters were adjusted for optimal signal detection of AlexaFluor488, AlexaFluor594 and AlexaFluor647 using a 2-sequence scanning approach. Z-stacks (7 μm with a 0.5 mm step size) were imaged using a confocal pinhole set at 1 airy unit

### Spatial Transcriptomics

### Tissue Preparation

Formalin-fixed paraffin-embedded tissue from Level 1 blocks was used for spatial transcriptomics analysis of engrafted PSC-CMs. Using a tissue microarray punch, the inventors collected 3 mm diameter samples of tissue from regions confirmed to contain PSC-CM grafts. The samples were melted down and re-embedded into four 6 mm×6 mm paraffin blocks, each containing 3 samples.

### RNA Quality Assessment

Three to five 7 m sections were collected per sample for RNA extraction using an RNeasy FFPE Kit (#73504, Qiagen). RNA Integrity Number (RIN) and DV200 were determined by BioAnalyzer using an RNA 6000 Pico Kit (#5067-1513, Agilent). DV200 is the percentage of total RNA fragments greater than 200 bp in length. All samples had DV200 from 46-57%.

### Tissue Optimization

To find optimal permeabilization time and tissue sectioning thickness, the inventors adapted the Visium Spatial Tissue Optimisation User Guide for fresh-frozen ST (CG000238 Rev A, 10× Genomics). FFPE blocks were sectioned at 7 m by rotary microtome. Paraffin sections were floated on nuclease-free water on 43° C. water bath for 2 min per section and captured while floating to each allocated array on the Visium Tissue Optimisation slides (#3000394). The slides with FFPE sections were then dehydrated with silica bead desiccants at room temperature for 1 hour, before overnight storage at 4° C. in a sealed slide-box containing silica beads. The following day, slide was dried at 37° C. for 15 minutes for complete dehydration, before wax melting at 60° C. for 60 minutes and deparaffinization by xylene (5 minutes, twice). Tissue rehydration was done using ethanol gradient (100% for 2 minutes, twice; 90% for 2 minutes, twice; 85% for 2 minutes). The above temperatures and times were optimised to ensure tissue adhere to the slide throughout the process.

Tissue sections were stained by haematoxylin and eosin (H&E) following the standard protocol and imaged at 20-40× magnification on a Zeiss AxioScan Z1 slide scanner. Next, tissues were decrosslinked by incubation with collagenase for 20 minutes at 37° C., then at 70° C. in TE buffer (pH 8.0) for 60 minutes. Immediately post decrosslinking, tissue sections were permeabilised by incubation with pepsin (0.1%) for different amounts of time (5 to 40 minutes). In this step, RNA was released from the tissue onto the glass slide, where the poly(A) tails hybridize to the slide-bound poly(dT) oligos. After permeabilization, reverse transcription reaction was performed to synthesise cDNA labelled with cyanine 3 (Cy3). Tissue sections were then removed from the slide using tissue removal buffer. The cDNA products on the slide were visualised on a Leica DMi8 inverted widefield microscope. By comparing fluorescence intensity and assessing diffusion of signal, the inventors determined the optimal permeabilization conditions to avoid over or under permeabilization.

### Sequencing Library Preparation

FFPE blocks were sectioned and placed onto a Visium Spatial Gene Expression Slide (#2000233, containing four capture areas), dried, dewaxed, deparaffinized, and stained as in the tissue optimisation procedure. After permeabilization, cDNA was synthesised using standard unlabelled nucleotides, followed by second strand synthesis. The spatial barcodes and unique molecular identifiers (UMI) are part of the oligos printed on the glass slide, and this sequence is incorporated to the first strand cDNA. The double stranded DNA products were denatured, and the released cDNA were PCR amplified for 18 cycles, end-repaired, A-tailed, and size-selected by SPRIselect (0.8× bead clean-up). Adapter ligation, PCR amplification and sample indexing followed Illumina TruSeq protocol. Final PCR amplification (10-15 cycles) and size selections (0.55× and 0.8× double-sided clean-up) were performed before QC by BioAnalyzer. The libraries were sequenced in a NovaSeq SP100 V1.5 kit (138 cycles), paired-end protocol as below: Read1—28 bp, Index1—10 bp, Index2—10 bp, Read2—90 bp.

### Sequencing Data Analysis

Raw NovaSeq BCL output file was converted to fastq file using SpaceRanger mkfastq V1.3.0 and bcl2fastq2-V2.17.1. The fastq files were processed further to remove adapter sequences and polyA sequences in the Read2, using cutadapt V3.2. A hybrid reference genome was made from combining Sscrofa11.1 (reference annotation Sscrofa11.1.105.gtf) with GRCh38-3.0.0 (genome build GRCh38.p12) using SpaceRanger mkref. The trimmed fastq reads were mapped to the hybrid genome using SpaceRanger count, which is based on STAR splicing-aware alignment. The gene expression matrix contains uniquely and confidently mapped UMIs. A UMI was only counted if it was mapped to a single exonic locus (at least 50% of the read intersect an exon), or if multiple mapping occurs, this UMI needs to have MAPQ255 (uniquely mapped), have bases 100% compatible with the exon of an annotated transcripts and aligned to the same strand. High resolution H&E images were used for the mapping of gene expression to spatial spots. The gene expression was used for down-stream analysis.

### Quality Control

It was a challenge to capture inter-species gene expression from a fixed engrafted tissue in a way that both the mRNA from human and pig could be measured. The inventors developed a protocol that allows the unbiased capture of the two species. The inventors noted that, the quality of the overall data was suboptimal. Therefore, a quality control process was applied to select the top two samples with the highest quality among all samples. Prior to downstream analysis, genes and spots that are likely noise or outliers were identified and removed. Across all samples, the inventors established a threshold on the number of genes detected (80) and the number of spots that contain a sufficient number of genes (10). This process resulted in two samples that passed the quality threshold, one with RA treatment (RA-PSC-CM #3) and the other with standard treatment (PSC-CM+CA #2). Each sample contained 1229 and 1107 human genes and 9105 and 5501 pig genes, from 808 and 567 spots for RA and standard treatment, respectively.

### Identification of Human and Pig Spots

For each spot, the inventors calculated its human score as the total number of reads that were mapped to the human genome, and its pig score as that mapped to the pig genome. In the RA treatment tissue, the inventors assigned a spot as human spot if its human score >30 and its pig score <700. In the standard treatment tissue, the inventors assigned a spot as human spot if its human score >30 and its pig score <350. In total the inventors identified 62 human spots in sample DZ24 and 50 in sample DZ22.

### Differential Expression Analysis

Although there was only one biological sample for each condition, the inventors utilised the spots which were measured separately from each other to perform pseudobulk differential gene expression analyses. For each sample, the inventors randomly pooled spots into three equally sized pools. For each pool, an average gene expression was calculated spot-wise. By doing this, the inventors created three pseudo-replicates for each condition, RA treatment vs standard treatment. With the pseudo-replicates, the inventors performed differential expression analysis following edgeR pipeline, using library size normalisation, and the quasi-likelihood test. Genes with adjusted p-value less than 0.05 were considered significant.

### Statistical Analyses

Continuous data are presented as mean±standard error of mean (SEM). Normality was assessed using the Shapiro-Wilk test, with appropriate parametric or non-parametric tests performed depending on distribution of data. Statistical comparisons of normally distributed data were conducted using unpaired t tests or ordinary one-way ANOVA followed by the Sidak's post hoc test to adjust for multiple comparisons. Non-normal data was statistically compared using the Mann-Whitney or Kruskal-Wallis test followed by Dunn's test to adjust for multiple comparisons. Correlations were expressed using Pearson's or Spearman's correlation coefficients. Inter- and intra-observer CMR analysis variability was expressed using Bland-Altman plots. Survival analyses were performed using the Kaplan-Meier method, and the log-rank test was applied to determine significance between overall survival between groups. P values <0.05 were considered statistically significant. All analyses were performed using GraphPad Prism Version 9.3.1 software.

### Results

### Bioreactor Production of PSC-CMs

To generate the requisite number of cardiomyocytes for porcine transplantation experiments, the inventors employed a stirred-tank bioreactor system for PSC expansion and differentiation. PSC-CM were generated following a three-stage process, using the H9 human embryonic stem cell line containing a gCaMP6f fluorescent calcium reporter (FIG. 1). Each batch of cardiomyocytes were generated from the same working cell bank (WCB) material. This was done to limit batch variability resulting from variations of the input population. The WCB was characterised for pluripotent markers, genetic stability, and differentiation capacity. After monolayer culture, cells were seeded into controlled environment stirred tank reactors (30% DO, pH 7.2, 37.2° C., 0.2-1.5 L volumes) for aggregate formation and pluripotent expansion over 3 days, consistently yielding aggregates between 100-200 μm in diameter. A thermoresponsive polymer, pNIPAAM, chemically linked to a recombinantly produced extracellular fragment of fibronectin (domains 7-10) was used as previously described (Chen, X. et al. Tissue Eng Part C Methods 24, 146-157) to aid aggregate formation. After heat shock and pre-treatment of aggregates with IGF-1 and Cyclosporin A, aggregates were single cell dissociated and cryopreserved on day 15. Assessment of cardiac troponin (cTnT) expression demonstrated an average of >80% cTnT⁺ cells and evidence of cytoskeletal formation. Additionally, genes for pluripotency, early mesoderm induction, and cardiac specification, were assessed via quantitative polymerase chain reaction (qPCR) over the time course of differentiation. These exhibited expression trends comparable to previously published PSC-CM production methods.

### PSC-CM Related Engraftment Arrhythmias are Focal and Automatic in Nature

After generation of sufficient PSC-CMs, the inventors conducted transplantation experiments over 3 phases in 23 landrace swine. Of these, 2 subjects died following induction of myocardial infarction and 1 died due to procedural complications following thoracotomy.

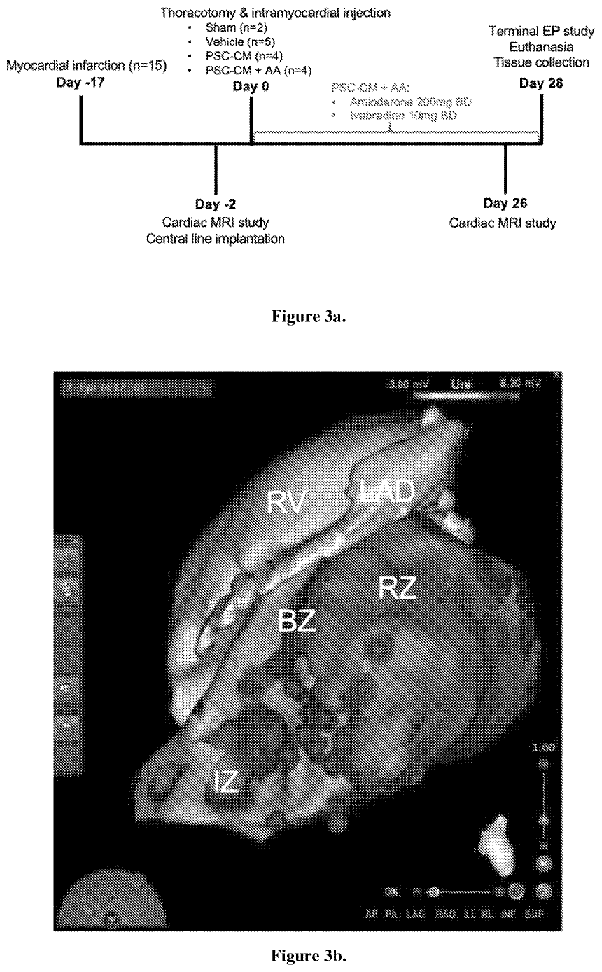

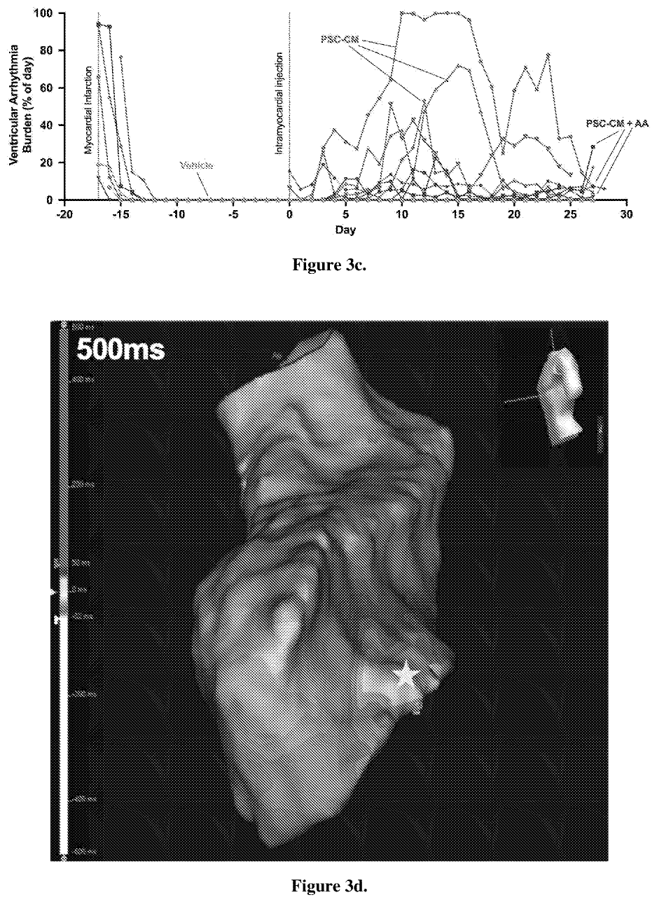

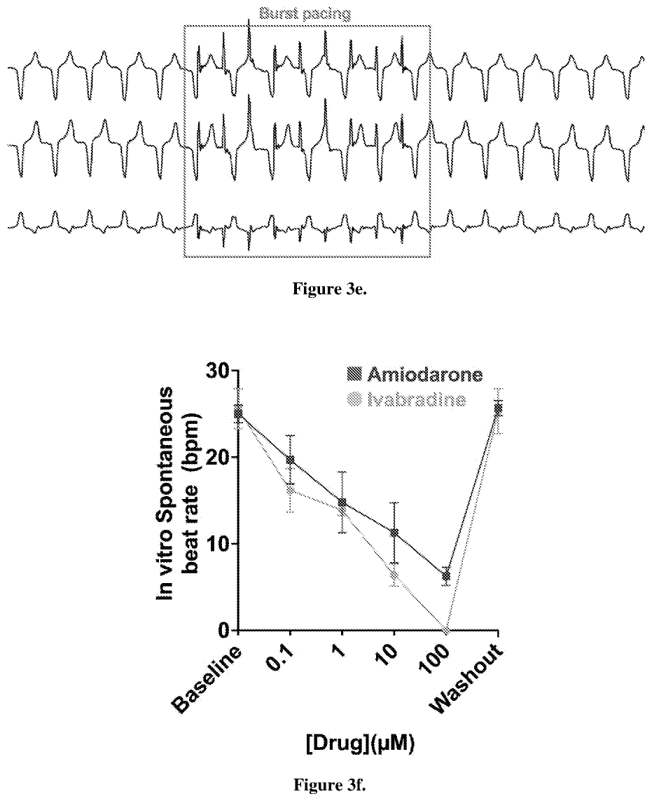

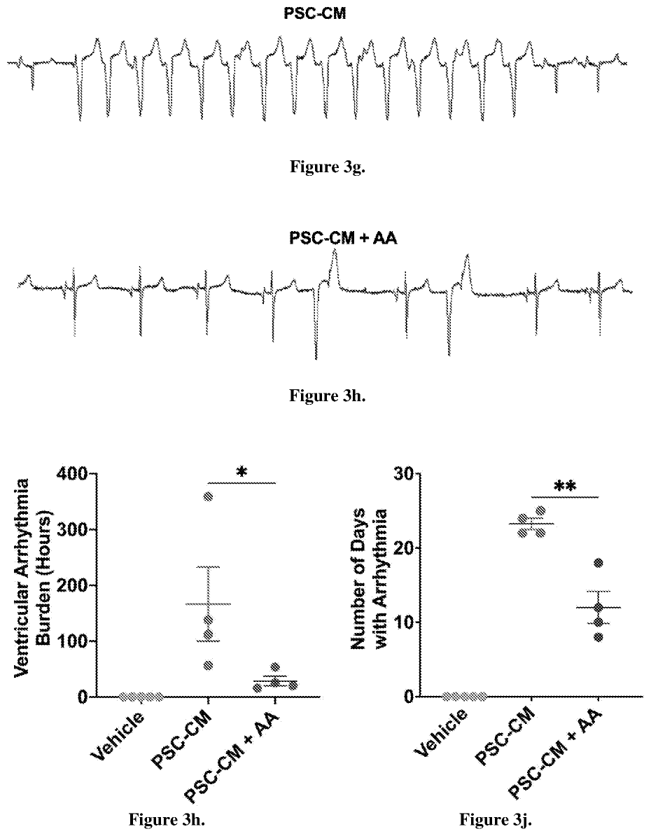

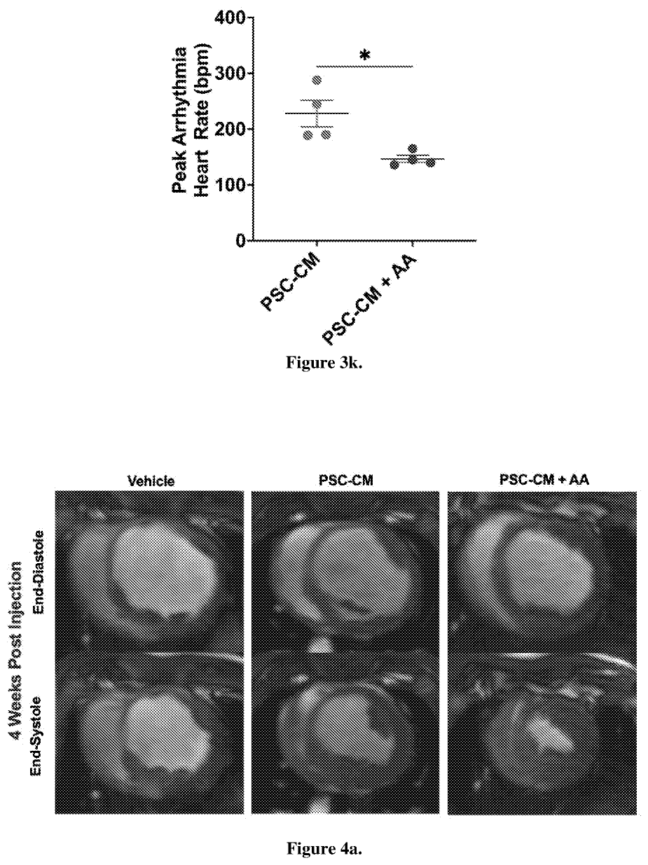

The first phase of the large animal experiments was designed to gain insights into the electrophysiological nature of PSC-CM related engraftment arrhythmias (EA) and to assess the treatment efficacy of clinically available anti-arrhythmic agents (AA). Transplantation studies were conducted in 15 subjects 2 weeks following percutaneously induced ischaemia-reperfusion myocardial infarction (FIG. 3a). Animals were randomised into 1 of 4 treatment groups: PSC-CM (n=4), PSC-CM+AA (n=4), vehicle (n=5), or sham infarction with vehicle injection (n=2). 750 million PSC-CMs or vehicle were delivered into infarct and border zones via transepicardial injections following lateral thoracotomy. To enable accurate targeting and annotation of cell injections, the inventors developed a novel technique in which epicardial voltage maps were merged with reconstructed cardiac magnetic resonance imaging (CMR) using a dedicated cardiac imaging platform, ADAS 3D (FIG. 3b). No spontaneous arrhythmias were observed in any vehicle treated subjects, however all cell treated subjects developed EA within a week of PSC-CM transplantation (FIG. 3c). Follow-up electroanatomic mapping studies performed 4 weeks post-transplantation localised EA origin to the focal sites of cell injection (FIG. 3d). Subsequent histological analyses confirmed these to be sites of cell engraftment.

In the same terminal mapping procedure, EA mechanism was elucidated by observing electrophysiological features and assessing arrhythmia response to pacing manoeuvres. All EAs occurred either spontaneously or after administration of the catecholaminergic drug isoprenaline. Termination was generally also spontaneous and EAs were not terminated by rapid ventricular pacing (FIG. 3e) nor after cardioversion by application of direct current (DC) to the external thorax. Cycle length (duration between consecutive electrocardiogram QRS complexes) variation of EAs was noted without changes in electrocardiogram (ECG) morphology. Together, these findings indicate enhanced automaticity as EA mechanism. This suggests PSC-CM grafts beat independently and more rapidly than the sinus node of recipient hearts. In contrast, vehicle treated animals only experienced arrhythmias which could be induced by rapid ventricular pacing, had fixed cycle lengths and were able to be terminated by rapid pacing or DC cardioversion. This suggests scar-mediated re-entrant circuits as the mechanism for induced arrhythmias in vehicle treated animals, the typical cause of post-MI ventricular arrhythmia.

### Effective Pharmacological Suppression of PSC-CM Related Engraftment Arrhythmias

Given the automatic mechanism for PSC-CM EAs, the inventors hypothesised that suppressing the rate of graft automaticity below that of the native sinus node would reduce arrhythmia burden. Ivabradine, a selective inhibitor of the pacemaker current responsible for cardiomyocyte automaticity, and amiodarone, a widely used anti-arrhythmic drug which blocks multiple ion channels, were selected as the anti-arrhythmic drugs. The inventors hypothesised the distinct mechanisms of action for each of these drugs would offer effective rate and rhythm control in treatment of the focal automatic EAs. In-vitro experiments confirmed that both these drugs induced dose-dependent reduction in the spontaneous beat rate of PSC-CMs cultured in monolayer (FIG. 3f). Pigs treated with PSC-CM+AA received a bolus of intravenous amiodarone at the time of cell injection followed by daily oral doses of amiodarone and ivabradine from day 1 to day 28 post cell injection. Arrhythmia detection was performed through blinded analysis of continuous ECG data transmitted from implanted telemetry units. Although all animals experienced transient arrhythmias attributable to reperfusion injury immediately following myocardial infarction, sinus rhythm was maintained for several days leading up to epicardial injections. Amiodarone-ivabradine treatment was extremely effective in suppressing EAs with clinically and statistically significant reduction of all telemetry parameters observed in drug treated pigs (FIG. 3g-k): total hours of arrhythmia (166.4±66.4 vs 28.6±8.6; p<0.05), days with arrhythmia (23.3±0.8 vs 12.0±2.2; p<0.005) and peak arrhythmia heart rate (228.0±23.9 beats per minute (b.p.m) vs 146.5±6.4 b.p.m; p<0.05). To further interrogate this finding the beat rates of sinus rhythm and EAs for each animal were analysed. EAs were noted to be slower in drug treated subjects (143.4±2.9 b.p.m vs 103.7±2.4; p<0.0001), with the difference between EA and SR beat rates significantly reduced in PSC-CM+AA subjects (39.2±2.1 b.p.m vs 19.7±2.3 b.p.m; p<0.0001). Together, these findings suggest that amiodarone-ivabradine treatment can successfully reduce PSC-CM graft automaticity leading to reduced EA burden with slower maximum rates.

PSC-CM with Anti-Arrhythmic Pharmacological Therapy Improves Left Ventricular Function Post-Myocardial Infarction

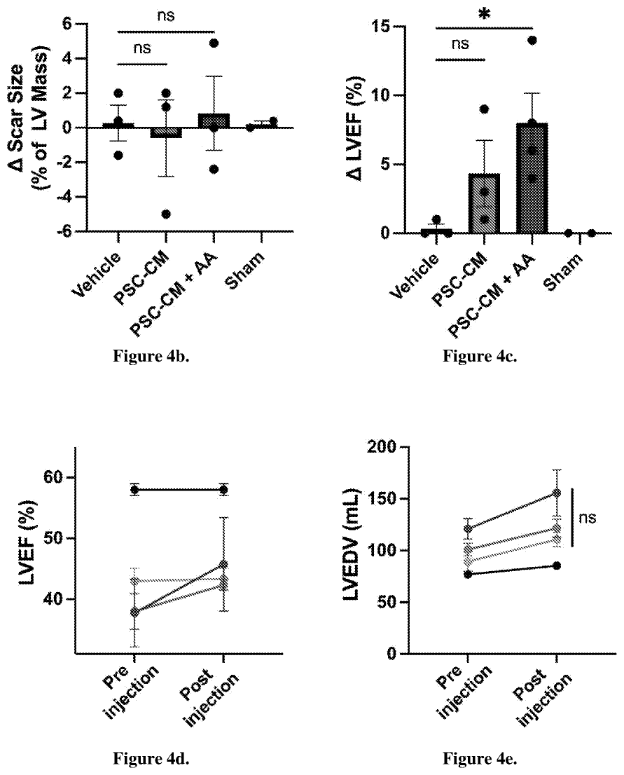

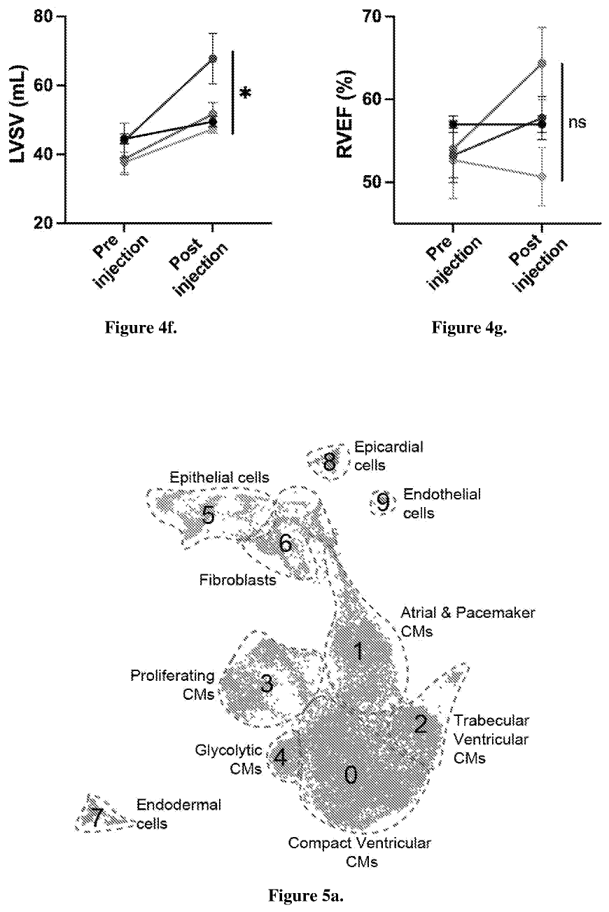

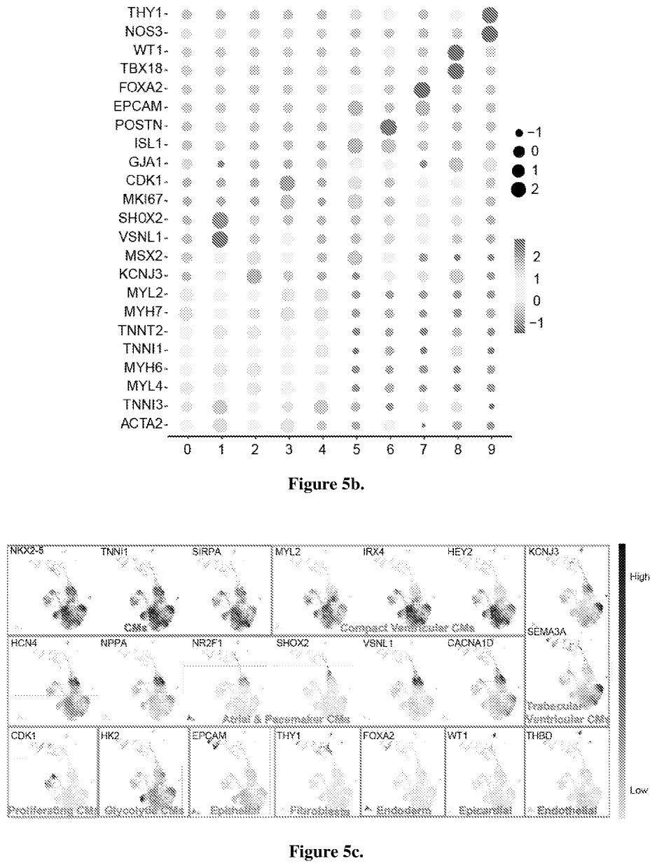

Although the primary goal of this study was not to demonstrate robust salutary effects with PSC-CM therapy, the inventors nonetheless assessed cardiac structure and function with serial CMR in cell and vehicle treated recipients. All subjects underwent CMR 2 days prior to and 4 weeks after transepicardial PSC-CM injections (FIG. 4a). Image analysis was conducted according to standard reporting guidelines by 2 blinded observers with excellent inter- and intra-observer variability. Three animals were excluded from functional analysis due to failure of infarct creation, all with scar sizes under 1.5% of left ventricular mass. Left ventricular ejection fraction (LVEF) was preserved in sham subjects (58±1%) and similarly depressed in all infarcted animals prior to transepicardial injection (Vehicle: 43±2%, PSC-CM: 38±3%, PSC-CM+AA: 38±6%; p=0.44, ns). At 4-weeks follow-up, no significant change in scar size, expressed as a percentage of total left ventricular mass was demonstrated between groups (change in scar size—Vehicle: 2±1%, PSC-CM: −0.6±2%, PSC-CM+AA: 0.8±2%; p<0.05; p=0.99) (FIG. 4b). Despite this, a statistically significant improvement in LVEF was observed (Change in LVEF—Vehicle: 0.3±0.3%, PSC-CM: 4±2%, PSC-CM+AA: 8±2%; p<0.05), with post-hoc analysis showing that this was driven by the PSC-CM+AA group (FIG. 4c-d). To further interrogate this finding the impact of the interventions on left ventricular volumes was evaluated. Despite improvement in stroke volume (LVSV) in the PSC-CM+AA subjects (Change in LVSV—Vehicle: 10±2 mL, PSC-CM: 13±1 mL, PSC-CM+AA: 24±4 mL; p<0.05), change in left ventricular end-diastolic volumes (LVEDV) were comparable between groups, and if anything, greater in PSC-CM+AA animals (Change in LVEDV—Vehicle: 21±4 mL, PSC-CM: 21±3 mL, PSC-CM+AA: 35±13 mL; p=0.38, ns) (FIG. 4e-f). Together, these data suggest the improvement in function observed in PSC-CM recipients may be attributable to greater left ventricular contractility rather than reduction of adverse remodelling and left ventricular dilation post-MI, with the greatest benefit evident in animals with arrhythmias suppressed by drug therapy.

Therapeutic efficacy on right ventricular function was also assessed. Despite not targeting right ventricular infarction, minor depression in right ventricular ejection fraction was noted in all infarcted animals (Sham: 57±1%, Vehicle: 53±5%, PSC-CM: 54±3%, PSC-CM+AA: 53±3%; p=0.96, ns). A trend to improvement was evident in all cell recipients, with this difference approaching, but not reaching statistical significance (Change in RVEF—Vehicle: −2±4%, PSC-CM: 10±3%, PSC-CM±AA: 5±3%; p=0.08, ns).

PSC-CM Cell Doses are Heterogeneous with Arrhythmogenic Subpopulations

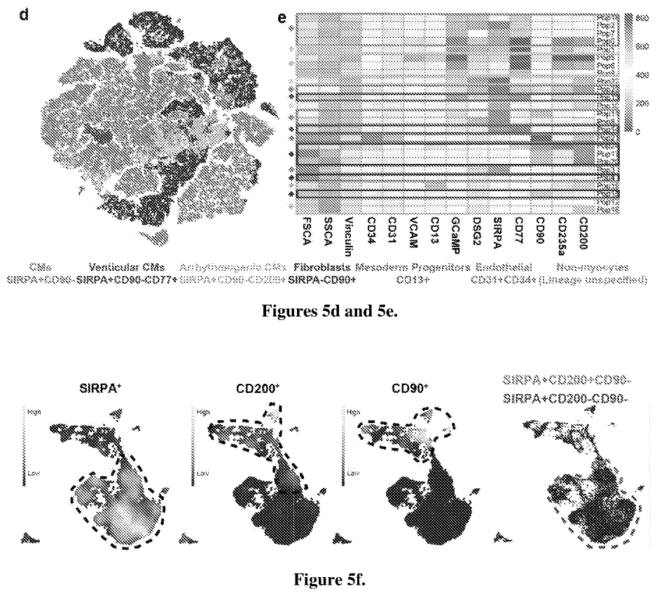

The inventors next sought to gain phenotypic insights into input PSC-CM composition to identify arrhythmogenic cellular characteristics that could be strategically targeted. Representative samples from each PSC-CM recipient's cell dose were retained prior to transplantation for use in single-cell RNA sequencing (scRNA-seq) and high dimensional flow cytometry experiments. Uniform manifold approximation and projection (UMAP) plots of clustered scRNA-seq data identified 10 distinct cellular subpopulations, the identities of which were surmised based on differential gene expression (FIG. 5a-c). The majority of cells expressed markers of a committed cardiac lineage such as NKX2-5, SIRPA and cTnT, representing clusters 0 to 4. Within these cardiomyocyte populations, further heterogeneity was noted. Compact ventricular markers such as MYL2, IRX4, MYH7 and HEY2 were most abundantly expressed in cluster 0 whereas atrial and pacemaker markers including SHOX2, VSNL1, NPPA and NR2F1 were tightly localised to cluster 1. Markers of trabecular myocardium such as KCNJ3, SEMA3A, IRX3 and SCN5A were predominantly expressed in cluster 2, with proliferative markers such as CDK1 and MKI67 identifying cluster 3. Cluster 4 showed strong expression of the glycolytic marker HK2, demarcating an earlier stage glycolytic cardiomyocyte population. Non-cardiomyocyte cell populations were also present with clusters 5-9 inclusive of fibroblasts, epithelial, endodermal, epicardial and endothelial cells. Together, this data confirms the dynamic transcriptional heterogeneity of profiled cells that were used in the described animal studies.

Cell dose characterisation by way of high-parameter flow cytometry also confirmed cellular heterogeneity (FIG. 5d-e). The inventors designed an antibody panel comprising of 12 surface markers to interrogate cell composition of the PSC-CMs delivered (Table 2). Resultant t-distributed stochastic neighbour embedding (tSNE) plots overlaid with 25 FlowSOM metaclusters were annotated where possible based on previously reported surface marker signatures. Of note, cardiomyocytes were classified as CD172a⁺/CD90⁻, with CD172a⁺/CD90⁻/CD77⁺ cells considered committed ventricular cardiomyocytes. Non-myocyte subpopulations were also present, with fibroblasts defined as CD172a⁻/CD90⁺ and endothelial cells defined as CD34⁺/CD31⁺. A small sub-population of CD13⁺ non-myocytes was identified, which may represent mesodermal progenitors. Due to the limitations of the surface marker panel in determining cellular fate several subpopulations could not be definitively labelled and were annotated as lineage unspecified cells. Results of single-cell RNA sequencing above (FIG. 5a) indicate these populations likely represent cells from epithelial and endodermal lineages.

To compare the relative abundance of specific subpopulations within and between cell doses, data from all doses were concatenated and analysed. This allowed subpopulation quantification which was then correlated with total arrhythmia burden for each cell recipient. PSC-CM+AA subjects were excluded from this analysis due to the confounding effect of arrhythmia suppression. Interestingly, the strongest correlation between subpopulation quantification and arrhythmia burden occurred with a previously undefined CD172a⁺/CD90⁻/CD200⁺ population (r=0.80). Conversely, CD172a⁺/CD90⁻/CD200⁻ cells held a negative association with arrhythmia burden (r=−0.77). To further define this surface marker signature scRNA-seq data was interrogated. CD172a⁺/CD90⁻/CD200⁺ cells isolated to cluster 1 (atrial and pacemaker cardiomyocytes) and CD172a⁺/CD90⁻/CD200⁻ to all remaining cardiomyocyte clusters (FIG. 5f). Taken together, these data confirm the cellular heterogeneity of transplanted PSC-CMs identifying a possible causal link between atrial and pacemaker like cardiomyocytes and arrhythmogenesis in PSC-CM treated subjects.

### Early Activation of Retinoic Acid Signalling During PSC-CM Differentiation Enriches for Atrial and Pacemaker-Like Subpopulations

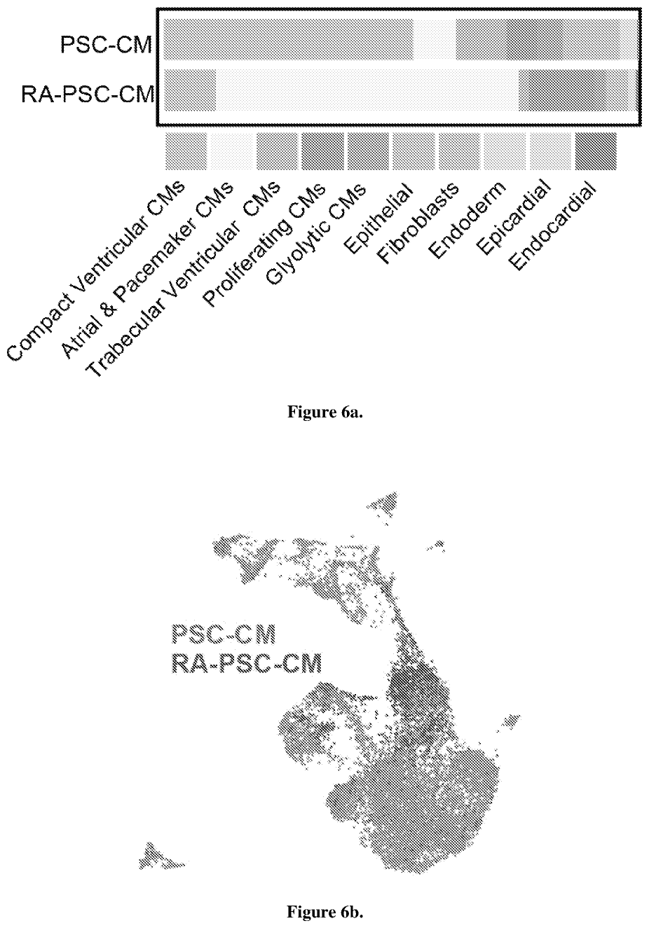

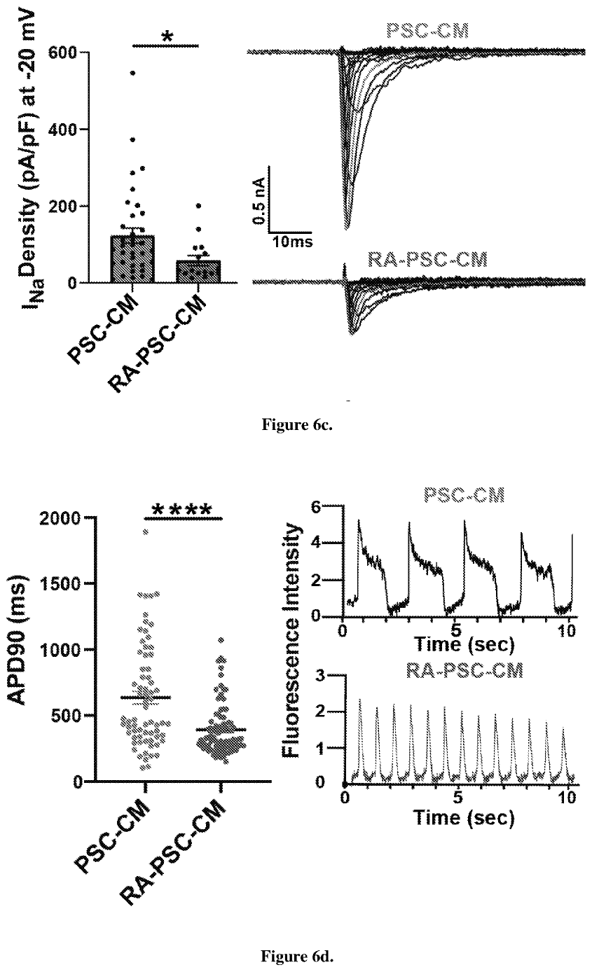

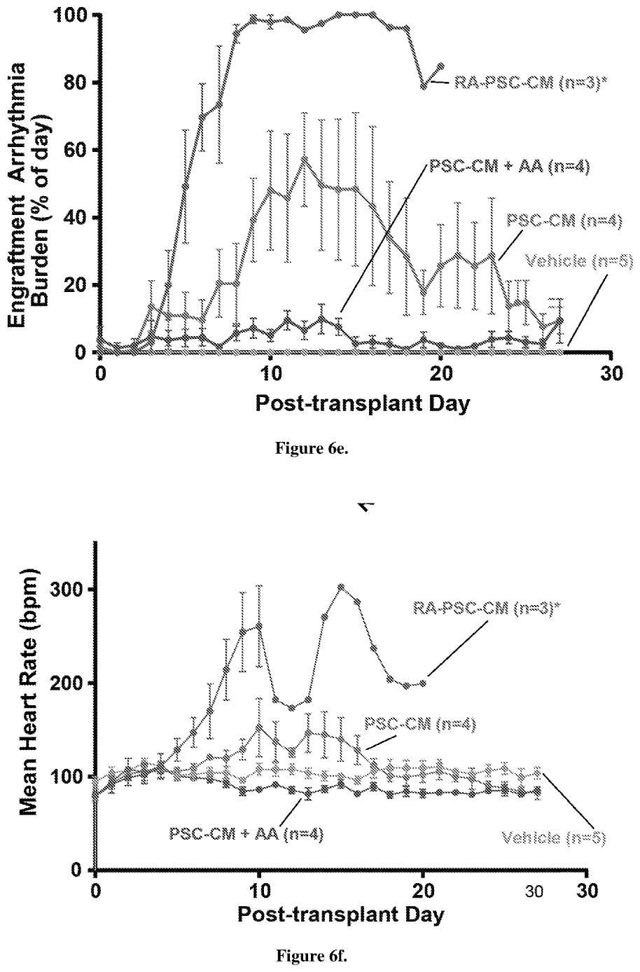

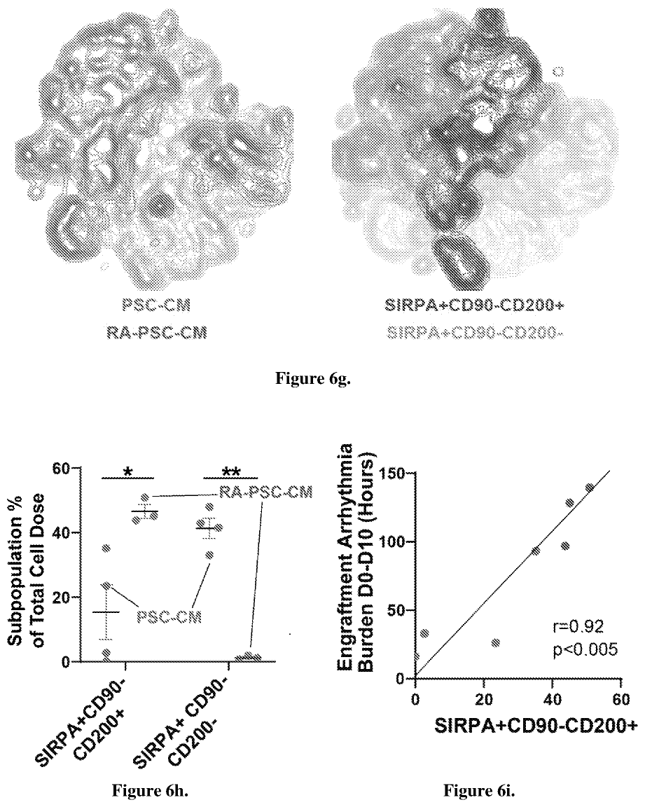

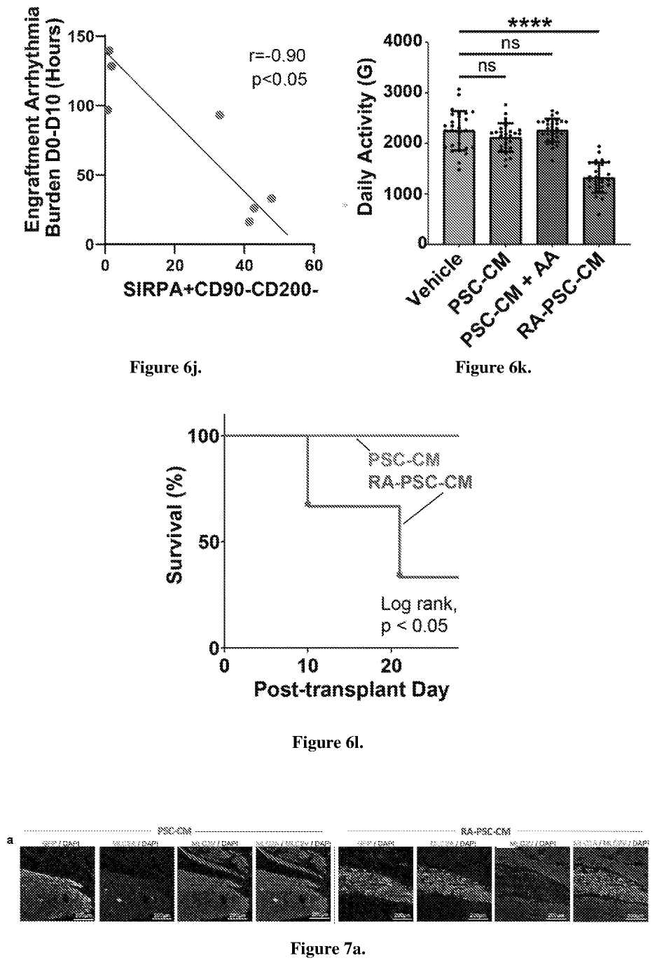

To further explore the arrhythmogenic potential of atrial and pacemaker like cardiomyocytes, the inventors developed a novel bioreactor differentiation protocol to enrich for these subpopulations. By modifying the standard protocol to activate the retinoic acid (RA) signalling pathway from day 2 to day 6, (FIG. 1) cardiac progenitors were directed to an atrial and pacemaker like fate (RA-PSC-CM). During CM manufacture, there was a reduction in cTnT expression when comparing RA treated conditions to control differentiations run in parallel (62.4±33.1% vs 83.6±7.0%, n=12). Importantly, expression of atrial and nodal genes such as NPPA, MYL2a, SHOX2 and HCN4 were upregulated as early as day 8 and significantly by day 15 in RA treated cultures, with converse suppression of ventricular markers such as IRX4 and MLC2v. This transcriptional pattern was confirmed with scRNA-seq analysis, in which RA-PSC-CMs showed a striking increase in atrial and pacemaker like cardiomyocytes and reduction of ventricular cardiomyocytes (FIG. 6a-b). Using the GCAMPf6\ reporter incorporated into the PSCs beat frequency was assessed on day 15 prior to aggregate dissociation. Retinoic acid treated aggregates had a significantly increased rate of spontaneous contraction (50.7±16.8 b.p.m, n=6 biological replicates with >20 aggregates counted per replicate) compared to the control (14.2±5.5 b.p.m, n=6). Electrophysiological analysis confirmed the phenotypic difference between standard and RA-PSC-CMs cell preparations with RA-PSC-CMs exhibiting faster spontaneous firing rates, reduced action potential durations and lower sodium current densities (FIG. 6c-d), features all consistent with atrial and pacemaker rather than ventricular cardiomyocytes.

RA-PSC-CM are Highly Arrhythmogenic after Transplantation into Infarcted Myocardium

To confirm whether RA-PSC-CM transplantation would augment in vivo arrhythmia burden, transplantation studies were conducted in 3 further infarcted porcine subjects in the second phase of the large animal experiments. These additional animals all received an equivalent dose of 750 million RA-PSC-CMs 2 weeks following myocardial infarction. A striking increase in arrhythmia burden and rate was noted in all 3 animals, who experienced rapid and near continuous EA by day 8 post cell delivery (FIG. 6e-f). High parameter flow cytometric analysis of each cell dose showed a greater proportion of the suspected arrhythmogenic CD172a⁺/CD90⁻/CD200⁺ subpopulation, and reduction in the non-arrhythmogenic CD172a⁺/CD90⁻/CD200⁻ subpopulation (FIG. 6g-h). Importantly, these additional animals significantly strengthened the positive (r=0.92, p<0.005) and negative (r=−0.90, p<0.05) arrhythmia correlation of these surface marker signatures (FIG. 6i-j), confirming the potential utility for these signatures in arrhythmia prediction. The elevated arrhythmia rate and burden was less favourably tolerated by the animals, who exhibited significantly reduced activity levels as quantified by accelerometer data (FIG. 6k). Unfortunately, 2 of the 3 RA-PSC-CM recipients succumbed to either heart failure related or arrhythmic deaths prior to completing their experimental time course (FIG. 6l), with the third surviving only due to timely intervention with catheter ablation, described further below.

RA-PSC-CM Grafts Contain Abundant Arrhythmogenic Subpopulations, with Reduced Sarcomeric Protein and Intercalated Disk Organisation in Comparison to PSC-CM Grafts

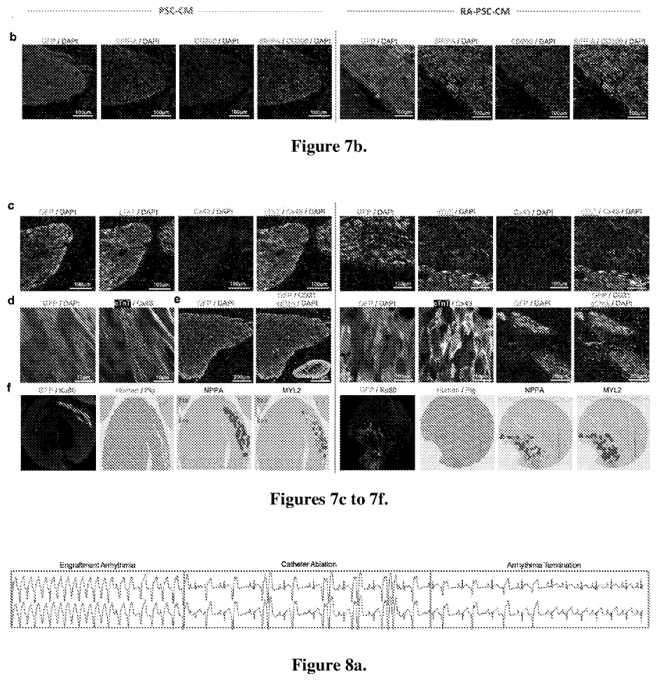

The fate of engrafted cells was also probed in histological and spatial transcriptomic experiments. Human graft was identified by staining with antibodies against the human nuclear antigen Ku80 or green fluorescence protein (GFP) within the GCAMP indicator of the cell line. For detection of ventricular, atrial, and pacemaker-like CMs within the graft, the inventors stained sections with antibodies targeting cardiac troponin T, MLC2v and MLC2a. Interestingly, RA-PSC-CM graft comprised strikingly more MLC2a⁺ cardiomyocytes suggesting greater atrial cardiomyocyte engraftment (FIG. 7a). In addition, these grafts also had a greater proportion of troponin⁺/MLC2v⁻ cardiomyocytes compared to standard PSC-CM graft, a signature suggestive of greater engrafted pacemaker cardiomyocytes. The inventors also interrogated the expression of CD172a⁺/CD200⁺ cardiomyocytes in standard versus RA-PSC-CM graft (FIG. 7b). Again, a striking difference was noted between both groups with RA-PSC-CM graft more abundant with arrhythmogenic CD172a⁺/CD200⁺ cardiomyocytes (FIG. 7b).

To visualise the organisation of sarcomeres and the formation of gap junctions between cardiomyocytes, the inventors stained grafts with antibodies against cardiac Troponin T (cTnT) and connexin 43 (Cx43) (FIG. 7c). High magnification confocal images show organised sarcomeres in standard PSC-CM grafts, with appropriate localisation of Cx43 to the intercalated disks (FIG. 7d). In stark contrast, RA-PSC-CM grafts had disorganised cTnT expression, with sporadic Cx43 compared to standard PSC-CM grafts, and lateralised expression pattern. The inventors also stained tissue with antibodies against CD31 and alpha smooth muscle actin, to identify new vessel growth within the grafts (FIG. 7e). Both standard PSC-CM and RA-PSC-CM grafts contained abundant host-derived microvessels (capillaries and arterioles) to support long-term graft survival.

Using an untargeted spatial transcriptomics protocol, the inventors captured polyA-tailed RNA from the grafted myocardium of one PSC-CM treated pig and one RA-PSC-CM treated pig. The inventors captured both human and pig RNA, resulting in the detection of 1107 and 1229 human genes and 5501 and 9105 pig genes, from 567 and 808 spatial spots of PSC-CM and RA-PSC-CM tissue, respectively. Spatial spots are uniquely barcoded hexagonal areas of 55 μM diameter which contained on average 1-9 cells. Based on the expression values of these genes, the inventors defined regions containing only pig cells (red dots) and spots containing only human cells (cyan dots) (FIG. 7f). The data-driven approach can automatically label human spots, which was independently confirmed by matching with the tissue regions containing human nuclear antigen, Ku80 (FIG. 7f). This is understood to be the first successful gene mapping of cells from either human or pig within an engrafted region. This mapping formed the basis for the inventors comparative analysis of transcriptional profiles between PSC-CM and RA-PSC-CM graft

Differential expression analysis of human spots identified three upregulated genes in RA-PSC-CM grafts, compared to PSC-CM. Two of these genes, NPPA (atrial natriuretic peptide) and MYH6 (myosin heavy chain, a isoform), are markers of atrial-like cardiomyocytes (FIG. 7f) and were also highly expressed in the atrial and pacemaker PSC-CM cluster identified using scRNAseq (FIG. 5a-c). The third upregulated gene, ELN (elastin), is an important component of the extracellular matrix. Genes for ventricular myosin isoforms (MYL2 and MYH7) and desmosomal components (DES, PKP2) were also found to be significantly downregulated in the RA-PSC-CM grafts.

Together, these data confirm the highly arrhythmogenic potential of atrial and pacemaker like cardiomyocytes abundant in RA-PSC-CM grafts, and identify CD172a⁺/CD90⁻/CD200⁺ and CD172a⁺/CD90⁻/CD200⁻ as novel surface marker signatures selecting for arrhythmogenic or non-arrhythmogenic cell preparations respectively.

### Catheter Ablation is a Feasible Alternative Treatment Strategy for PSC-CM Related Engraftment Arrhythmias

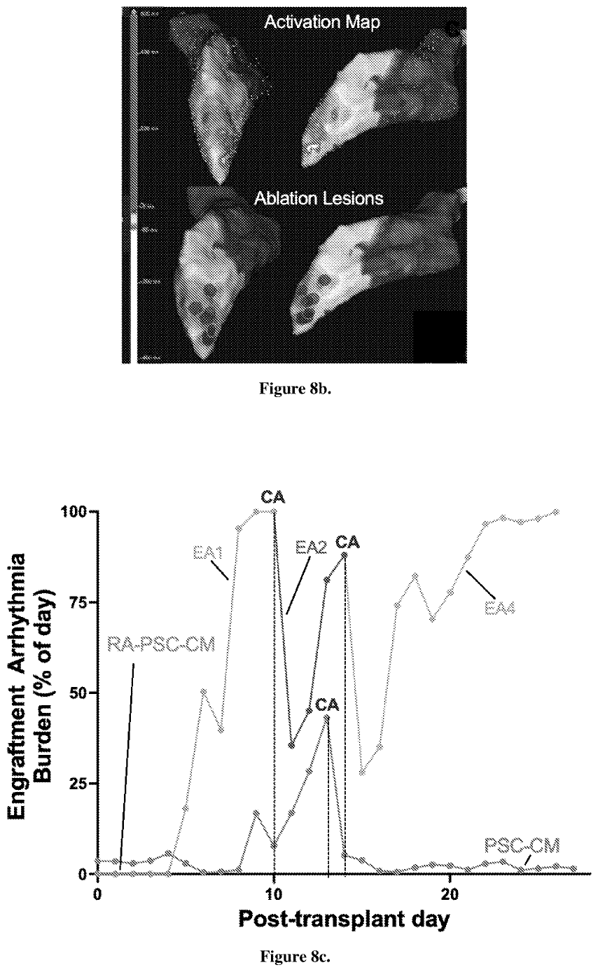

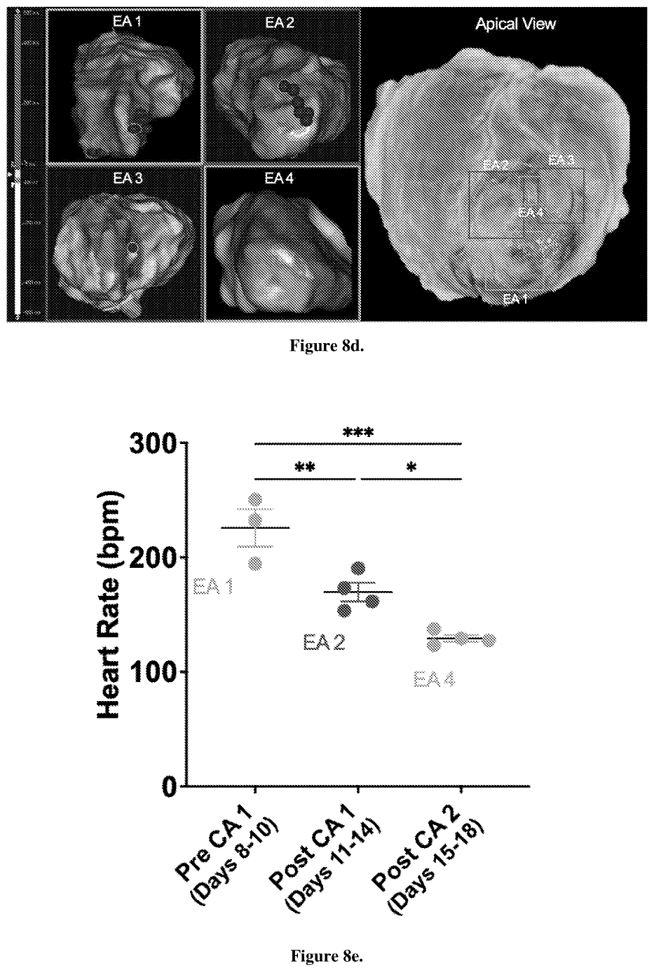

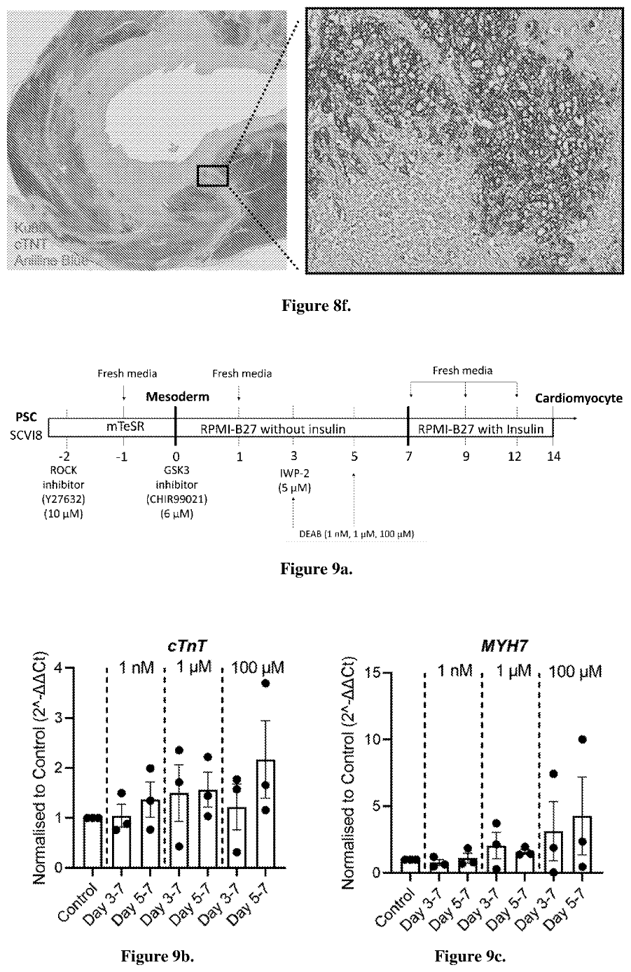

Despite the demonstration of amiodarone-ivabradine suppressing EA burden, a fall-back strategy in the case of aggressive or refractory EAs is imperative for safe PSC-CM clinical translation. Thus, in the third and final phase of the large animal studies, the inventors sought to determine the feasibility and efficacy of catheter ablation (CA) as an alternative EA treatment strategy. CA is an advanced procedural option for treating cardiac arrhythmias, in which arrhythmic foci are electroanatomically identified then disrupted, typically with radiofrequency energy delivered through a specialised catheter. A further 2 pigs underwent PSC-CM delivery following myocardial infarction, with the intention of proceeding to CA 2 weeks post cell injection. Only one of these subjects displayed sufficient EA to facilitate electroanatomic mapping and ablation, with inadequate burden in the second. ScRNA-seq for the latter showed minimal contribution of atrial and pacemaker-like cardiomyocytes in the input cell dose with flow cytometry showing a high percentage of non-arrhythmogenic CD172a⁺/CD90⁻/CD200⁻ cardiomyocytes. Together these findings account for the low arrhythmia burden in this subject. In the first subject, EA burden was trending upward by day 13, at which point electroanatomic mapping and CA was performed. This localised EA origin to the inferolateral apex, and this region was targeted with a series of ablations terminating EA and restoring sinus rhythm (FIG. 8a-b). EA did not recur intraoperatively despite a period of monitoring and aggressive arrhythmia induction strategies (programmed electrical stimulation, isoprenaline infusion, burst pacing). Telemetry analysis from the subsequent 2 weeks showed a marked drop in EA burden (FIG. 8c) with only isolated ventricular ectopic beats rather than sustained arrhythmias noted.

After this successful proof-of-concept experiment, the inventors sought to assess the efficacy of CA in pigs treated with the highly arrhythmogenic RA-PSC-CMs. Only 1 of the 3 pigs treated with RA-PSC-CM underwent CA, with the remaining 2 animals dying before planned ablation procedures could take place. In the treated animal, CA was a lifesaving intervention facilitating survival through to the terminal time-point. However, two procedures were required with a greater number of ablation lesions and higher radiofrequency doses required for arrhythmia termination compared to the standard PSC-CM pig. In addition, EA recurred within 12 hours after each procedure despite apparent initial procedural success (FIG. 8c). Interestingly, recurrent EA was always found to originate from a new site, correlating with a separate cell injection location. In total, 4 different origins of EAs were identified, 3 of which (EAs 1-3) were successfully ablated. The final arrhythmia (EA4) was mapped at the terminal procedure but not ablated due to the pre-determined endpoint being reached and euthanasia following the mapping procedure was conducted as planned. Examination of the heart following tissue harvest demonstrated excellent anatomic correlation with preceding endocardial activation maps with clearly visible transmural ablation lesions identifying EA1-3 along with an island of surviving cell graft identifying EA4 (FIG. 8d). Of interest, despite recurrence following each ablation, the new arrhythmias were of lower heart rates (FIG. 8e-f) and more favourably tolerated by the animal on clinical assessment. Histological analysis confirmed successful disruption of engrafted regions with each ablation, with intact, surviving graft responsible for the residual arrhythmia (FIG. 8g).

Taken together, these results indicate that CA is a feasible therapeutic strategy for EA, however PSC-CM grafts may demonstrate hierarchical pacemaker-like attributes with ectopic EA activity falling back to alternative graft sites once the dominant graft has been ablated. Treatment success, particularly with highly arrhythmogenic cell doses, may require complete ablation of all engrafted regions if these cell populations are not removed prior to transplantation.

### DISCUSSION

The inventors have shown that PSC-CM related EAs can be suppressed and potentially abolished with clinically available pharmacological and procedural therapeutics. The inventors further identify cellular characteristics of arrhythmogenic PSC-CMs through robust phenotyping of input and engrafted cells. These data can inform cell production strategies to provide safe and effective PSC-CMs for future clinical trials.

The inventors have shown that the combination of ivabradine and amiodarone is extremely effective in suppressing graft automaticity as well as reducing EA rate and burden. Ivabradine was selected given its specific inhibition of the I_{f} current responsible for pacemaker automaticity, a characteristic the inventors hypothesised may be effective in suppressing automatic EAs. Conversely, amiodarone was selected given it is an established anti-arrhythmic agent which may be empirically administered in future PSC-CM clinical trials. It has a broad mechanism of action, antagonising K⁺, Na⁺ and Ca²⁺ channels along with β-adrenergic receptors. The combination of these drugs effectively suppressed but did not eradicate EAs, necessitating assessment of alternate treatment strategies particularly in the case of highly arrhythmogenic cell doses. Therefore, the inventors went on to show that catheter ablation (CA) using radiofrequency energy application to arrhythmogenic foci identified by electroanatomic mapping is a feasible strategy to treat and abolish EA. This proof-of-concept experimental result has important practical implications as the first patients are being treated with PSC-CMs. Interestingly, the ablation experiments also yielded important EA mechanistic insights, demonstrating for the first time that pacemaker activity can fall back to grafts of lower intrinsic pacemaker rates once the dominant graft has been ablated. A caveat to CA therapy for EA is that ablating multiple graft regions risks loss of the contractile benefits exerted by PSC-CM transplantation. As such, the inventors propose CA only be pursued in the case of aggressive and pharmacologically refractory EAs and more prudently, that generation of less arrhythmogenic PSC-CM cell products be investigated. To this end, the inventors also sought to gain mechanistic insights into the cellular compositions which may contribute to EAs.

Substantial variability of EA burden has been reported within and between PSC-CM large animal studies. Though this may be reflective of several factors such as differences in animal species or cell delivery technique, it is likely to be predominantly determined by the molecular make-up of the cellular product. Reductions in arrhythmia burden have been noted after a period of in vivo graft maturation, suggesting PSC-CM immaturity may be a key determinant of arrhythmogenesis. However, not all PSC-CM recipients achieve ‘electrical maturation’, implying immaturity may not be the only important cellular characteristic. Current PSC-CM differentiation protocols are known to generate heterogeneous cell populations containing a mix of ventricular, atrial and pacemaker like cardiomyocytes. For the first time, the inventors outline the importance of this heterogeneity in arrhythmogenesis, identifying atrial and pacemaker-like cells as culprit subpopulations along with describing unique surface marker signatures predictive of arrhythmogenicity. Cells enriched for atrial and pacemaker-like cardiomyocytes were generated through addition of retinoic acid to the bioreactor differentiation protocol and shown to be highly arrhythmogenic supporting a causal link between these subpopulations and EA. The inventors show that the enhanced automaticity of these subpopulations in vitro directly translates to more abundant and rapid EAs in vivo. Furthermore, by robustly phenotyping input cell doses and correlating these to resultant arrhythmia burden, the inventors identify CD172a⁺/CD90⁺/CD200⁺ and CD172a⁺/CD90⁻/CD200⁻ cells as arrhythmogenic and non-arrhythmogenic cardiomyocytes respectively. In doing so, the inventors not only provide a simple quality control tool for assessing the arrhythmogenic potential of cell doses, but also an avenue for removing arrhythmogenic cells by cell sorting prior to transplantation. Interestingly, the scRNA-seq data shows these arrhythmogenic CD172a⁺/CD90⁺/CD200⁺ cardiomyocytes have a transcriptomic signature consistent with atrial and pacemaker subpopulations, further supporting the notion that these cell types are important in arrhythmogenesis. There is currently substantial interest in generating chamber-specific cardiomyocytes for various therapeutic or drug discovery applications. This data further endorses the pursuit of transplanting purified ventricular cardiomyocytes, devoid of atrial and pacemaker-like cells for the purposes of therapeutic cardiac remuscularisation.

Finally, although not the primary intention and despite small sample sizes, the inventors were able to demonstrate left ventricular functional improvement in all cell recipients but particularly those in those which EAs had been ameliorated with pharmacotherapy. PSC-CM grafts elicit direct contractile force with the data suggesting that this beneficial effect can be further enhanced once arrhythmogenicity is addressed. Interestingly, a trend to improvement was also noted in the ungrafted right ventricle, suggesting immunomodulatory paracrine influences may also be a factor.

In conclusion, repopulation of infarcted myocardium with functional, force generating cardiomyocytes is an exciting therapeutic prospect positioning PSC-CMs as a leading candidate for cardiac regeneration. Though associated with transient arrhythmogenesis, here the inventors deepen the mechanistic understanding of this predictable complication, informing that it is likely addressable through modifications to cardiomyocyte production protocols. Additionally, the inventors show that PSC-CM engraftment arrhythmias can be suppressed with clinically available pharmacologic and procedural anti-arrhythmic strategies, an important safety consideration given several impending clinical trials.

### DEAB PSC-CM Cultures

### Methods

hPSC SCVI8 Culture

hPSC SCVI8 were cultured in Matrigel® (Corning, Cat. No. 354277) coated petri dishes in complete mTeSR™ Plus basal medium (StemCell Technologies, Cat. No. 100-0276) supplemented with 20% (v/v) mTeSR™ Plus 5× supplement (StemCell Technologies) and 0.5% (v/v) streptomycin and penicillin (ThermoFisher, Cat. No. 15140163) at 37° C. in 5% CO₂. Media was changed daily and cells passaged when confluency reached approximately 80%.

### Cardiomyocyte Differentiation

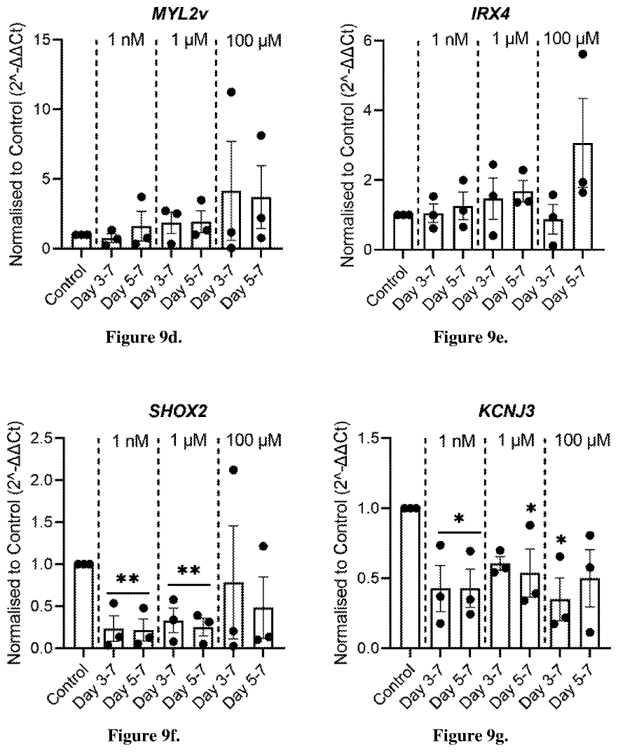

Cardiomyocyte differentiation was achieved using small-molecule modulators of canonical Wnt signalling as described by Lian et al. (Lian et al. Nature. 2013; 8(1):162-175), but with the addition of 1 nM, 1 μM or 100 μM 4-diethylaminobenzaldehyde (DEAB, Sigma Merck, Cat. No. D86256) at either day 3 or 5 of differentiation (FIG. 9a.). In brief, hPSC SCVI8 cells from confluent dishes were washed once with Dulbeco's Phosphate Buffered Saline (DPBS) without calcium and magnesium (Lonza, Cat. No. 17-512F), and harvested with TrypLE™ Express Enzyme (ThermoFisher, Cat. No. 12604021) and seeded at a concentration of 3.75×10⁵ cells/mL in Matrigel® coated 12-well plates at a volume of 2 mL of complete mTeSR Plus basal medium and 10 μM Y-27632 (RHO/ROCK) inhibitor (StemCell Technologies, Cat. No. 072304). At day 0 of differentiation, cells were washed once using DPBS and transferred to mesoderm induction media that consisted of 6 μM CHIR99021 (GSK3 inhibitor, Tocris, Cat. No. 4423) in complete Roswell Park Memorial Institute (RPMI) 1640 medium, no glutamine (ThermoFisher, Cat. No. 21870092) supplemented with 2% (v/v) B-27 supplement, minus insulin (ThermoFisher, Cat. No. A1895601), 0.5% (v/v) streptomycin and penicillin and 1% (v/v) GlutaMAX™ supplement (ThermoFisher, Cat. No. 35050061). At day 1, CHIR99021 was removed, and cells maintained in complete RPMI minus insulin. At day 3, cells were transferred to cardiac induction media that consisted of 5 μM IWP-2 (Tocris, Cat. No. 3533) in complete RPMI minus insulin and was removed at day 5. At day 7 of differentiation, cells were maintained in complete RPMI supplemented with 2% (v/v) B-27™ Supplement (50×) (ThermoFisher, Cat. No. 17504-001), 0.5% (v/v) streptomycin and penicillin and 1% (v/v) GlutaMAX™ supplement. The media was changed every 2-3 days. Cells were harvested at day 14 for quantitative real-time polymerase chain reaction (RT-PCR).

### Quantitative Reverse Transcription Polymerase Chain Reaction (RT-PCR)

Total cellular RNA was extracted using the Isolate II RNA Mini Kit (Bioline, Cat. No. BIO-52073) as per the manufacturer's instructions. Reverse transcription was performed with M-MLV Reverse Transcriptase (Promega, Cat. No. M1701) following the manufacturer's protocol. mRNA gene expression was assessed by qPCR using SensiFAST™ SYBR® No-ROX Kit (Meridian Bioscience, Cat. No. BIO-98050) on the Bio-Rad CFX384 Real-Time PCR detection system, with the following PCR conditions: 95° C. for 2 min and 40 cycles of 95° C. for 5 s, 60° C. for 10 s and 72° C. for 15 s, followed by melt curve analysis. All primers used for detection of mRNA expression are outline in Table 3. All data was normalised to the reference gene 18S and normalised to control cells (non-treated PSC-CM).

TABLE 3
Genes and primers used for qPCR experiments.

| Gene | Forward Primer | Reverse Primer |
|---|---|---|
| 18S | AAACGGCTACCACATCCAAG | CCTCCAATGGATCCTCGTTA |
| cTnT | AGTGGGAAGAGGCAGACTGA | CGAACTTCTCTGCCTCCAAG |
| MYH7 | TCCATGTATAAGCTGACAGGC | CCATGAGGTAGGCAGACTTG |
| MYL2v | GACCCTGAGGAAACCATTCT | CAGCATTTCCCGAACGTAATC |
| IRX4 | TTGGACTCCTGGGAACATGGACAA | ATGCTTCAGGGTATCTGGCCTCTT |
| SHOX2 | AAGAGGATGCGAAAGGGATG | TGAGTTGTTCCAGGGTGAAAT |
| KCNJ3 | TCATCAAGATGTCCCAGCCCAAGA | CACCCGGAACATAAGCGTGAGTTT |

### Statistical Analysis

All statistical tests were performed using Prism 9.00 software (GraphPad Software Inc). Details of the tests performed are given in the figure legends.

### Results

Differentiating hPSCs were treated with DEAB during day 3 to 7 or day 5 to 7 of differentiation at concentrations ranging from 1 nM-100 μM (FIG. 9a.). RT-PCR analysis of PSC-CM at day 14 demonstrated no significant difference in the expression levels of the cardiac specific gene cTnT (FIG. 9b.) as well as the ventricular markers MYH7, MYL2v and IRX4 (FIG. 9c.-e.) across the different treatment groups. However, there were significant lower expression levels of SHOX2 (FIG. 9f.), a gene associated with SAN pacemaker and KCNJ3 (FIG. 9g.), a pacemaker ion channel gene in PSC-CM cells treated with DEAB.

The non-significant trend of cTnT, MYH7 and MLC2v expression indicates that ventricular cardiomyocytes are enriched, while atrial/pacemaker cells depleted (see KCNJ3 and SHOX2 data). It can be seen from FIG. 5 that the CD172a+/CD90−/CD200− subpopulation does not express SHOX2 and KCNJ3. Therefore it is expected that PSC-CM treated with DEAB during differentiation are enriched in the CD172a+/CD90−/CD200− subpopulation. It can also be seen from FIG. 5 that the CD172a+/CD90−/CD200+ subpopulation has increased expression of SHOX2, thus PSC-CM treated with DEAB during differentiation are expected to be depleted in the CD172a+/CD90−/CD200+ subpopulation.

PSC-CM Cultures with Removal of Vitamin A

PSC-CM cultures are prepared in culture media having reduced vitamin A and in culture media free from vitamin A. Removal of vitamin A may be important two days following Wnt inhibition.

An exemplary protocol is as follows, where the chemically defined medium (CDM) is RPMI 1640 basal medium, L-ascorbic acid 2-phosphate and bovine serum albumin (BSA). The CDM is supplemented with B27, which is available both with and without vitamin A,
Day 0=CHIR99021 addition (CDM B27 minus insulin, including vitamin A)
Day 1=media change (CDM B27 minus insulin, including vitamin A)
Day 3=IWP-2 addition (CDM B27 minus insulin, including vitamin A)
Day 5=media change (CDM B27 plus insulin, no vitamin A)
Day 7=media change (CDM B27 plus insulin, no vitamin A)

An alternative exemplary protocol is as follows:
Day 0=CHIR99021 addition (RPMI B27 minus insulin, including vitamin A)
Day 3=IWP-2 addition (RPMI B27 minus insulin, including vitamin A, retinoic acid inhibitor)
Day 5=media change (CDM B27 plus insulin, no vitamin A)

An alternative exemplary protocol is as follows:
Day 0=CHIR99021 addition (CDM B27 minus insulin, including vitamin A)
Day 3=IWP-2 addition (CDM B27 minus insulin, including vitamin A)
Day 5=media change (CDM B27 plus insulin, no vitamin A, plus BMS inhibitor)
Day 7=media change (CDM B27 plus insulin, no vitamin A, plus BMS inhibitor)

The invention is further exemplified in the following numbered paragraphs:
1. A method of identifying an arrhythmogenic pluripotent stem cell-derived cardiomyocyte (PSC-CM), the method comprising:
   (i) determining whether CD200 is expressed on a surface of the PSC-CM;
wherein if CD200 is expressed on the surface of the PSC-CM, the PSC-CM is an arrhythmogenic PSC-CM.
2. The method of paragraph 1, further comprising:
   (ii) determining whether Signal regulatory protein α (CD172a) is expressed on the surface of the PSC-CM, and/or
   (iii) determining whether CD90 is expressed on the surface of the PSC-CM;
wherein if CD200 is expressed on a surface of the PSC-CM, and CD172a is expressed on the surface of the PSC-CM and/or CD90 is not expressed on the surface of the PSC-CM, the PSC-CM is an arrhythmogenic PSC-CM.
3. The method of paragraph 1, further comprising:
   (ii) determining whether CD172a is expressed on the surface of the PSC-CM, and
   (iii) determining whether CD90 is expressed on the surface of the PSC-CM;
wherein if CD200 and CD172a are expressed on the surface of the PSC-CM and CD90 is not expressed on the surface of the PSC-CM, the PSC-CM is an arrhythmogenic PSC-CM.
4. The method of any one of paragraphs 1 to 3, wherein if CD200 is not expressed on the surface of the PSC-CM, the PSC-CM is a non-arrhythmogenic PSC-CM.
5. The method of any one of paragraphs 1 to 4, wherein determining whether CD200, CD172a and/or CD90 is expressed on the surface of the PSC-CM comprises exposing the PSC-CM to an agent comprising a detectable label to provide a labelled PSC-CM, and detecting the detectable label, wherein the agent selectively binds to CD200, CD172a or CD90.
6. The method of paragraph 5, wherein determining whether CD200, CD172a and CD90 are expressed on the surface of the PSC-CM comprises exposing the PSC-CM to a first agent comprising a first detectable label that selectively binds to CD200, a second agent comprising a second detectable label that selectively binds to CD172a, and a third agent comprising a third detectable label that selectively binds to CD90, to provide a labelled PSC-CM, and detecting the detectable labels.
7. The method of paragraph 5 or paragraph 6, wherein the agent comprising a detectable label is an antibody, and the detectable label is a fluorophore.
8. The method of any one of paragraphs 5 to 7, wherein detecting the detectable label comprises subjecting the labelled hPSC-CM to flow cytometry.
9. The method of any one of paragraphs 1 to 8, wherein the PSC-CM is a human pluripotent stem cell-derived cardiomyocyte (hPSC-CM).
10. A method of determining whether a dose containing a plurality of PSC-CMs is likely to cause arrhythmia upon transplantation in a subject, the method comprising:
   (i) determining whether the dose contains arrhythmogenic PSC-CMs, wherein whether a PSC-CM is an arrhythmogenic PSC-CM is determined according to the method of any one of paragraphs 1 to 9;
wherein if the dose contains arrhythmogenic PSC-CM's, the dose is likely to cause arrhythmia upon transplantation in the subject.
11. The method of paragraph 10, further comprising:
   (ii) determining the proportion of arrhythmogenic PSC-CMs in the dose, wherein if the proportion of PSC-CMs in the dose which express CD200 on a surface thereof is greater than 0.001%, the dose is likely to cause arrhythmia upon transplantation in the subject.
12. A method of eliminating or reducing arrhythmia following transplantation of a dose containing a plurality of PSC-CMs in a subject, the method comprising, prior to transplantation of the dose:
   (i) identifying arrhythmogenic PSC-CMs contained in a crude dose according to the method of any one of paragraphs 1 to 9; and
   (ii) removing the arrhythmogenic PSC-CMs from the crude dose to obtain a purified dose.
13. The method of paragraph 12, further comprising:
   (iii) transplanting the purified dose in the subject.
14. The method of paragraph 12 or paragraph 13, wherein the crude dose comprises pluripotent stem cells (PSCs) which have been differentiated to form PSC-CMs, and have not been subject to any additional treatments enriching or depleting a cell subpopulation.
15. The method of any one of paragraphs 12 to 14, wherein arrhythmia following transplantation of the purified dose is reduced compared to arrhythmia following transplantation of the crude dose.
16. The method of paragraph 15, wherein the arrhythmia is reduced as defined by cumulative time per day spent in arrhythmia over 25 days post-transplantation.
17. The method of paragraph 16, wherein arrhythmia is reduced by at least about 50%, or at least about 60, 70, 80, 90, 95, or 100%.
18. The method of any one of paragraphs 12 to 17, wherein step (ii) comprises removing at least about 50% of the arrhythmogenic PSC-CMs from the crude dose.
19. The method of any one of paragraphs 12 to 18, wherein step (ii) comprises removing at least about 60%, or at least about 70, 80, 90, 95, or 100% of the arrhythmogenic PSC-CMs from the crude dose
20. The method of any one of paragraphs 12 to 19, wherein step (ii) is performed by fluorescence-activated cell sorting or by magnetic-activated cell sorting.
21. A method of providing a dose containing a plurality of PSC-CM which is substantially free from arrhythmogenic PSC-CMs, the method comprising:
   (i) identifying arrhythmogenic PSC-CMs contained in a crude dose according to the method of any one of paragraphs 1 to 9; and
   (ii) removing the arrhythmogenic PSC-CMs from the crude dose to obtain a dose of PSC-CM which is substantially free from arrhythmogenic PSC-CMs.
22. The method of paragraph 21, wherein the crude dose comprises human pluripotent stem cells (PSCs) which have been differentiated to form PSC-CMs, and have not been subject to any additional treatments enriching or depleting a cell subpopulation.
23. The method of paragraph 21 or paragraph 22, wherein step (ii) comprises removing at least about 50% of the arrhythmogenic PSC-CMs from the crude dose.
24. The method of any one of paragraphs 21 to 23, wherein step (ii) comprises removing at least about 60%, or at least about 70, 80, 90, 95, or 100% of the arrhythmogenic PSC-CMs from the crude dose.
25. The method of any one of paragraphs 21 to 24, wherein the dose of PSC-CM which is substantially free from arrhythmogenic PSC-CMs contains less than 0.001% of arrhythmogenic PSC-CMs, relative to the total number of PSC-CM in the dose.
26. The method of any one of paragraphs 21 to 25, wherein step (ii) is performed by fluorescence-activated cell sorting or by magnetic-activated cell sorting.
27. A method of identifying a pluripotent stem cell-derived cardiac cell with pacemaker properties (PSC-PM) the method comprising:
   (i) determining whether CD200 is expressed on a surface of a pluripotent stem cell-derived cardiac cell;
wherein if CD200 is expressed on the surface of the pluripotent stem cell-derived cardiac cell, the pluripotent stem cell-derived cardiac cell is a PSC-PM.
28. The method of paragraph 27, further comprising:
   (ii) determining whether CD172a is expressed on the surface of the pluripotent stem cell-derived cardiac cell, and/or
   (iii) determining whether CD90 is expressed on the surface of the pluripotent stem cell-derived cardiac cell;
wherein if CD200 is expressed on a surface of the pluripotent stem cell-derived cardiac cell, and CD172a is expressed on the surface of the pluripotent stem cell-derived cardiac cell and/or CD90 is not expressed on the surface of the pluripotent stem cell-derived cardiac cell, the pluripotent stem cell-derived cardiac cell is a pacemaker PSC-PM.
29. The method of paragraph 27 or paragraph 28, further comprising:
   (ii) determining whether CD172a is expressed on the surface of the pluripotent stem cell-derived cardiac cell, and
   (iii) determining whether CD90 is expressed on the surface of the pluripotent stem cell-derived cardiac cell;
wherein if CD200 and CD172a are expressed on the surface of the pluripotent stem cell-derived cardiac cell and CD90 is not expressed on the surface of the pluripotent stem cell-derived cardiac cell, the pluripotent stem cell-derived cardiac cell is a PSC-PM.
30. The method of any one of paragraphs 27 to 29, wherein if CD200 is not expressed on the surface of the pluripotent stem cell-derived cardiac cell, the pluripotent stem cell-derived cardiac cell is not a PSC-PM.
31. The method of any one of paragraphs 27 to 30, wherein determining whether CD200, CD172a and/or CD90 is expressed on the surface of the pluripotent stem cell-derived cardiac cell comprises exposing the pluripotent stem cell-derived cardiac cell to an agent comprising a detectable label to provide a labelled pluripotent stem cell-derived cardiac cell, and detecting the detectable label, wherein the agent selectively binds to CD200, CD172a or CD90.
32. The method of paragraph 31, wherein determining whether CD200, CD172a and CD90 are expressed on the surface of the pluripotent stem cell-derived cardiac cell comprises exposing the pluripotent stem cell-derived cardiac cell to a first agent comprising a first detectable label that selectively binds to CD200, a second agent comprising a second detectable label that selectively binds to CD172a, and a third agent comprising a third detectable label that selectively binds to CD90, to provide a labelled pluripotent stem cell-derived cardiac cell, and detecting the detectable labels.
33. The method of paragraph 31 or paragraph 32, wherein the agent comprising a detectable label is an antibody, and the detectable label is a fluorophore.
34. The method of any one of paragraphs 31 to 33, wherein detecting the detectable label comprises subjecting the labelled pluripotent stem cell-derived cardiac cell to flow cytometry.
35. A method of providing a substantially pure dose of PSC-PM, the method comprising:
   (i) identifying PSC-PMs contained in a plurality of pluripotent stem cell-derived cardiac cells according to the method of any one of paragraphs 27 to 34; and
   (ii) isolating the PSC-PMs from the plurality of pluripotent stem cell-derived cardiac cells to obtain the substantially pure dose of PSC-PM.
36. The method of paragraph 35, wherein step (ii) is performed by fluorescence-activated cell sorting or by magnetic-activated cell sorting.
37. The method of paragraph 35 or paragraph 36, wherein the substantially pure dose of PSC-PM contains less than 0.001% of cells which are not PSC-PM, relative to the total number of cells in the dose.
38. A method of identifying a pluripotent stem cell-derived ventricular cardiomyocyte (PSC-VM), the method comprising:
   (i) determining whether CD200 is expressed on a surface of a pluripotent stem cell-derived cardiac cell;
wherein if CD200 is not expressed on the surface of the pluripotent stem cell-derived cardiac cell, the pluripotent stem cell-derived cardiac cell is a PSC-VM.
39. The method of paragraph 38, further comprising:
   (ii) determining whether CD172a is expressed on the surface of the pluripotent stem cell-derived cardiac cell, and/or
   (iii) determining whether CD90 is expressed on the surface of the pluripotent stem cell-derived cardiac cell;
wherein if CD200 is not expressed on a surface of the pluripotent stem cell-derived cardiac cell, and CD172a is expressed on the surface of the pluripotent stem cell-derived cardiac cell and/or CD90 is not expressed on the surface of the pluripotent stem cell-derived cardiac cell, the pluripotent stem cell-derived cardiac cell is a PSC-VM.
40. The method of paragraph 38 or paragraph 39, further comprising:
   (ii) determining CD172a is expressed on the surface of the pluripotent stem cell-derived cardiac cell, and
   (iii) determining whether CD90 is expressed on the surface of the pluripotent stem cell-derived cardiac cell;
wherein if CD200 and CD90 are not expressed on the surface of the pluripotent stem cell-derived cardiac cell and CD172a is expressed on the surface of the pluripotent stem cell-derived cardiac cell, the pluripotent stem cell-derived cardiac cell is a PSC-VM.
41. The method of any one of paragraphs 38 to 40, wherein if CD200 is expressed on the surface of the pluripotent stem cell-derived cardiac cell, the pluripotent stem cell-derived cardiac cell is not a PSC-VM.
42. The method of any one of paragraphs 37 to 41, wherein determining whether CD200, CD172a and/or CD90 is expressed on the surface of the pluripotent stem cell-derived cardiac cell comprises exposing the pluripotent stem cell-derived cardiac cell to an agent comprising a detectable label to provide a labelled pluripotent stem cell-derived cardiac cell, and detecting the detectable label, wherein the agent selectively binds to CD200, CD172a or CD90.
43. The method of paragraph 42, wherein determining whether CD200, CD172a and CD90 are expressed on the surface of the pluripotent stem cell-derived cardiac cell comprises exposing the pluripotent stem cell-derived cardiac cell to a first agent comprising a first detectable label that selectively binds to CD200, a second agent comprising a second detectable label that selectively binds to CD172a, and a third agent comprising a third detectable label that selectively binds to CD90, to provide a labelled pluripotent stem cell-derived cardiac cell, and detecting the detectable labels.
44. The method of paragraph 42 or paragraph 43, wherein the agent comprising a detectable label is an antibody, and the detectable label is a fluorophore.
45. The method of any one of paragraphs 42 to 44, wherein detecting the detectable label comprises subjecting the labelled pluripotent stem cell-derived cardiac cell to flow cytometry.
46. A method of providing a substantially pure dose of PSC-VM, the method comprising:
   (i) identifying PSC-VM contained in a plurality of pluripotent stem cell-derived cardiac cells according to the method of any one of paragraphs 37 to 44; and
   (ii) isolating the PSC-VM from the plurality of pluripotent stem cell-derived cardiac cells to obtain the substantially pure dose of PSC-VM.
47. The method of paragraph 46, wherein step (ii) is performed by fluorescence-activated cell sorting or by magnetic-activated cell sorting.
48. The method of paragraph 46 or paragraph 47, wherein the substantially pure dose of PSC-PM contains less than 0.001% of cells which are not PSC-PM, relative to the total number of cells in the dose.
49. A method of eliminating or reducing arrhythmia following transplantation of a dose containing a plurality of PSC-CMs in a subject, the method comprising culturing a plurality of PSCs in a culture medium under conditions effective to suppress the generation of arrhythmogenic PSC-CMs, wherein an arrhythmogenic PSC-CM is a PSC-CM which expresses CD200 on a surface thereof.
50. The method of paragraph 49, wherein arrhythmia following transplantation of a dose subjected to the method of paragraph 49 is reduced compared to arrhythmia following transplantation of a dose which is not subjected to the method of paragraph 49.
51. The method of paragraph 50, wherein arrhythmia is reduced as defined by cumulative time per day spent in arrhythmia over 25 days post-transplantation.
52. The method of paragraph 51, wherein arrhythmia is reduced by at least about 50%, or at least about 60, 70, 80, 90, 95, or 100%.
53. A method of providing a dose of PSC-CM which is substantially free from arrhythmogenic PSC-CMs, the method comprising culturing PSCs in a culture medium under conditions effective to suppress the generation of arrhythmogenic PSC-CMs, wherein an arrhythmogenic PSC-CM is a PSC-CM which expresses CD200 on a surface thereof.
54. The method of paragraph 53, wherein the dose of PSC-CM which is substantially free from arrhythmogenic PSC-CMs contains less than 0.001% of arrhythmogenic PSC-CMs, relative to the total number of PSC-CM in the dose.
55. The method of any one of paragraphs 49 to 54, wherein an arrhythmogenic PSC-CMs is a PSC-CM which expresses CD200, and expresses CD172a and/or does not express CD90, on the surface thereof.
56. The method of any one of paragraphs 49 to 55, wherein an arrhythmogenic PSC-CM is a PSC-CM which expresses CD200 and CD172a, and does not express CD90, on the surface thereof.
57. A method of providing a dose of PSC-CM which contains an increased proportion of non-arrhythmogenic PSC-CMs, the method comprising culturing PSCs in a culture medium under conditions effective to increase the generation of non-arrhythmogenic PSC-CMs, wherein a non-arrhythmogenic PSC-CM is a PSC-CM which does not express CD200 on a surface thereof.
58. The method of paragraph 57, wherein the dose of PSC-CM which contains an increased proportion of non-arrhythmogenic PSC-CMs contains at least about 5% more non-arrhythmogenic PSC-CMs, relative to the total number of PSC-CM in the dose, and compared to a control dose which is obtained by culturing PSCs in a culture medium in the absence of conditions effective to increase the generation of non-arrhythmogenic PSC-CMs.
59. The method of paragraph 57 or paragraph 58, wherein a non-arrhythmogenic PSC-CMs is a PSC-CM which does not express CD200, and does not express CD90 and/or expresses CD172a, on the surface thereof.
60. The method of any one of paragraphs 57 to 59, wherein a non-arrhythmogenic PSC-CM is a PSC-CM which does not express CD200 and CD90, and expresses CD172a on the surface thereof.
61. The method of any one of paragraphs 49 to 60, wherein the conditions effective to suppress the generation of arrhythmogenic PSC-CMs comprise addition of a retinoic acid inhibitor to the culture medium or removal of vitamin A from the culture medium.
62. The method of paragraph 61, wherein the retinoic acid inhibitor is added during cardiomyocyte differentiation.
63. The method of paragraph 62, wherein the retinoic acid inhibitor is added at between day 3 to day 7 of cardiomyocyte differentiation.
64. The method of any one of paragraphs 61 to 63, wherein the retinoic acid inhibitor is selected from the group consisting of 4-diethylaminobenzaldehyde (DEAB), 4-[(1E)-2-[5,6-dihydro-5,5-dimethyl-8-(2-phenylethynyl)-2-naphthalenyl]ethenyl]benzoic acid (BMS-493), (E)-4-[2-(5,6-dihydro-5,5-dimethyl-8-phenyl-2-naphthalenyl)ethenyl]-benzoic acid (BMS-189453), and disulfiram.
65. The method of paragraph 61, wherein vitamin A is removed from the culture medium during cardiomyocyte differentiation.
66. A purified population of non-arrhythmogenic pluripotent stem cell-derived cardiomyocytes (PSC-CMs), wherein the population of non-arrhythmogenic PSC-CMs does not express CD200 on the cell surface.
67. The purified population of paragraph 66, wherein the population does not express CD172a on the cell surface.
68. The purified population of paragraph 66 or 67, wherein the population does express CD90 on the cell surface.
69. A purified population of non-arrhythmogenic PSC-CMs, wherein the population is prepared by
   (i) providing a population of PSC-CMs;
   (ii) determining whether CD200 is expressed on the cell surface of the PSC-CMs in step (i);
   (iii) optionally determining whether CD172a and/or CD90 is expressed on the cell surface of the PSC-CMs in step (i);
   (iv) removing the PSC-CMs from step (i) that express CD200, and optionally removing PSC-CMs that express CD172 and/or do not express CD90,
   thereby preparing a purified population of non-arrhythmogenic PSC-CMs.
70. A method of providing a PSC-CM graft to a patient in need thereof, comprising administering a population of non-arrhythmogenic PSC-CMs of any one of paragraphs 66-69 to the patient, wherein the patient is at a lower risk of engraftment arrhythmia (EA) than a patient receiving a mixed population of PSC-CMs.
71. A method of ameliorating engraftment arrhythmia (EA) in a patient that has received a transplant of PSC-CMs comprising treating the patient with amiodarone and/or ivabradine.
72. A method of ameliorating EA in a patient that has received a transplant of PSC-CMs comprising performing at least one catheter ablation on the patient.

1. A purified population of non-arrhythmogenic pluripotent stem cell-derived cardiomyocytes (PSC-CMs), wherein the population of non-arrhythmogenic PSC-CMs does not express CD200 on the cell surface.

2. A purified population of non-arrhythmogenic PSC-CMs, wherein the population is prepared by
(i) providing a population of PSC-CMs;
(ii) determining whether CD200 is expressed on the cell surface of the PSC-CMs in step (i);
(iii) optionally determining whether CD172a and/or CD90 is expressed on the cell surface of the PSC-CMs in step (i);
(iv) removing the PSC-CMs from step (i) that express CD200, and optionally removing PSC-CMs that express CD172 and/or do not express CD90,
thereby preparing a purified population of non-arrhythmogenic PSC-CMs.

3. A method of providing a PSC-CM graft to a patient in need thereof, comprising administering a population of non-arrhythmogenic PSC-CMs of claim 1 or 2 to the patient, wherein the patient is at a lower risk of engraftment arrhythmia (EA) than a patient receiving a mixed population of PSC-CMs.

4. A method of identifying an arrhythmogenic pluripotent stem cell-derived cardiomyocyte (PSC-CM), the method comprising:
(i) determining whether CD200 is expressed on a surface of the PSC-CM;
wherein if CD200 is expressed on the surface of the PSC-CM, the PSC-CM is an arrhythmogenic PSC-CM.

5. A method of determining whether a dose containing a plurality of PSC-CMs is likely to cause arrhythmia upon transplantation in a subject, the method comprising:
(i) determining whether the dose contains arrhythmogenic PSC-CMs, wherein whether a PSC-CM is an arrhythmogenic PSC-CM is determined according to the method of claim 4;
wherein if the dose contains arrhythmogenic PSC-CM's, the dose is likely to cause arrhythmia upon transplantation in the subject.

6. A method of eliminating or reducing arrhythmia following transplantation of a dose containing a plurality of PSC-CMs in a subject, the method comprising, prior to transplantation of the dose:
(i) identifying arrhythmogenic PSC-CMs contained in a crude dose according to the method of claim 4; and
(ii) removing the arrhythmogenic PSC-CMs from the crude dose to obtain a purified dose.

7. A method of providing a dose containing a plurality of PSC-CM which is substantially free from arrhythmogenic PSC-CMs, the method comprising:
(i) identifying arrhythmogenic PSC-CMs contained in a crude dose according to the method of claim 4; and
(ii) removing the arrhythmogenic PSC-CMs from the crude dose to obtain a dose of PSC-CM which is substantially free from arrhythmogenic PSC-CMs.

8. A method of identifying a pluripotent stem cell-derived cardiac cell with pacemaker properties (PSC-PM) the method comprising:
(i) determining whether CD200 is expressed on a surface of a pluripotent stem cell-derived cardiac cell;
wherein if CD200 is expressed on the surface of the pluripotent stem cell-derived cardiac cell, the pluripotent stem cell-derived cardiac cell is a PSC-PM.

9. A method of providing a substantially pure dose of PSC-PM, the method comprising:
(i) identifying PSC-PMs contained in a plurality of pluripotent stem cell-derived cardiac cells according to the method of claim 8; and
(ii) isolating the PSC-PMs from the plurality of pluripotent stem cell-derived cardiac cells to obtain the substantially pure dose of PSC-PM.

10. A method of identifying a pluripotent stem cell-derived ventricular cardiomyocyte (PSC-VM), the method comprising:
(i) determining whether CD200 is expressed on a surface of a pluripotent stem cell-derived cardiac cell;
wherein if CD200 is not expressed on the surface of the pluripotent stem cell-derived cardiac cell, the pluripotent stem cell-derived cardiac cell is a PSC-VM.

11. A method of providing a substantially pure dose of PSC-VM, the method comprising:
(i) identifying PSC-VM contained in a plurality of pluripotent stem cell-derived cardiac cells according to the method of claim 10; and
(ii) isolating the PSC-VM from the plurality of pluripotent stem cell-derived cardiac cells to obtain the substantially pure dose of PSC-VM.

12. A method of eliminating or reducing arrhythmia following transplantation of a dose containing a plurality of PSC-CMs in a subject, the method comprising culturing a plurality of PSCs in a culture medium under conditions effective to suppress the generation of arrhythmogenic PSC-CMs, wherein an arrhythmogenic PSC-CM is a PSC-CM which expresses CD200 on a surface thereof.

13. A method of providing a dose of PSC-CM which is substantially free from arrhythmogenic PSC-CMs, the method comprising culturing PSCs in a culture medium under conditions effective to suppress the generation of arrhythmogenic PSC-CMs, wherein an arrhythmogenic PSC-CM is a PSC-CM which expresses CD200 on a surface thereof.

14. A method of providing a dose of PSC-CM which contains an increased proportion of non-arrhythmogenic PSC-CMs, the method comprising culturing PSCs in a culture medium under conditions effective to increase the generation of non-arrhythmogenic PSC-CMs, wherein a non-arrhythmogenic PSC-CM is a PSC-CM which does not express CD200 on a surface thereof.

15. A method of ameliorating engraftment arrhythmia (EA) in a patient that has received a transplant of PSC-CMs comprising treating the patient with amiodarone and/or ivabradine.

16. A method of ameliorating EA in a patient that has received a transplant of PSC-CMs comprising performing at least one catheter ablation on the patient.
